(19) United States
(12) Patent Application Publication
(10) Pub. No.: US 2024/0148868 A1
(43) Pub. Date: May 9, 2024

(54) CHIMERIC PHAGOCYTIC RECEPTORS FOR TREATMENT OF NEURODEGENERATIVE DISORDERS

(71) Applicant: University of Florida Research Foundation, Incorporated, Gainesville, FL (US)

(72) Inventors: Todd Eliot Golde, Gainesville, FL (US); Marshall S. Goodwin, Gainesville, FL (US); Brenda Dawn Moore, Gainesville, FL (US); Thomas B. Ladd, Gainesville, FL (US); Yong Ran, Gainesville, FL (US); Yona Levites, Gainesville, FL (US); Pedro Cruz, Gainesville, FL (US)

(73) Assignee: University of Florida Research Foundation, Incorporated, Gainesville, FL (US)

(21) Appl. No.: 18/282,503

(22) PCT Filed: Mar. 17, 2022
(86) PCT No.: PCT/US2022/020824

§ 371 Date: Sep. 15, 2023

Related U.S. Application Data
(60) Provisional application No. 63/163,016, filed on Mar. 18, 2021.

(51) Int. Cl. A61K 39/00; A61K 48/00; A61P 25/28; C07K 14/705; C07K 16/18; C12N 15/86

(52) U.S. Cl. CPC A61K 39/4614; A61K 39/4633; A61K 39/4643; A61K 48/0058; A61P 25/28; C07K 14/70503; C07K 16/18; C12N 15/86; C07K 2317/622; C07K 2319/02; C07K 2319/03; C07K 2319/43; C12N 2750/14143

(57) ABSTRACT

Provided herein are chimeric receptors, engineered cells and pharmaceutical compositions for enhancement of long-term clearance of protein aggregates in the central nervous system via phagocytosis or engulfment. Further provided herein are methods of treatment of subjects suffering from, or diagnosed with, a neurodegenerative disease by administering these receptors, modified cells, and/or pharmaceutical compositions. Administration of one or more, or a plurality of these modified cells, to a subject may provide treatment of a neurodegenerative disease such as Alzheimer's Disease (AD) or Parkinson's Disease (PD). Advantageously, the modified cells, pharmaceutical compositions, and methods of the present disclosure meet existing needs in the art by providing clearance of accumulations of protein aggregates in brain tissue in PD and AD pathologies.

### BACKGROUND OF THE INVENTION

Many neurodegenerative disorders, including Alzheimer's disease (AD), are hypothesized to be proteinopathies, or neurodegenerative disorders characterized by the misfolding and/or aggregation of specific proteins within neurons or glial cells of the brain. Despite the growing understanding of the pathogenic mechanisms underlying these disorders, progress with respect to development of new therapeutics has been lagging. Based on the premise that protein aggregation and accumulation in AD and many other neurodegenerative disorders is a key, causal, pathologic event, it is logical to pursue therapeutic strategies to i) prevent aggregate formation, and ii) enhance clearance of the aggregate or iii) neutralize “toxic” signaling by the aggregate.

Many strategies targeting protein aggregates have been developed, but these strategies have proven difficult to translate, or in some cases, found to have dose limiting side-effects. Further, even in the preclinical studies supporting clinical development, most of these therapeutic approaches have shown modest effects. Robust effects may have been reported from prophylactic or early intervention studies, but evidence for efficacy is much more limited, or absent, for treatment studies initiated when pathology is widespread.

Overall, effective therapies for Alzheimer's Disease (AD), Parkinson's Disease (PD) and related neurodegenerative diseases remains a significant unmet medical need. Immunotherapeutic approaches remain a major focus of the effort to develop effective disease modifying therapeutics for AD. As opposed to inhibitors that target amyloid-beta (Aβ, or Abeta) plaque production in the brain, anti-Aβimmunotherapies have potential to clear preexisting deposits, neutralize toxic Aβ aggregates, or both. Thus, there is better rationale for testing immunotherapies in patients with preexisting pathology.

### SUMMARY OF THE INVENTION

The present disclosure provides novel chimeric receptors, modified cells, pharmaceutical compositions, and methods for enhancement of long-term clearance of protein aggregates in the central nervous system (CNS) via phagocytic mechanisms. Provided herein are compositions of engineered cells expressing a chimeric phagocytic receptor on the cell surface that may be administered to a subject in need thereof. These engineered cells may be administered through autologous or allogeneic mechanisms.

The present disclosure is based, at least in part, on the understanding that enhancing clearance of the target protein via phagocytic mechanisms is the most direct way to clear toxic proteins within the CNS. This disclosure provides novel chimeric phagocytic receptors (CPRs) to achieve this clearance. Multiple CPRs having affinity for a target protein selected from Aβ, alpha-synuclein, and tau have been engineered and functionally validated. The CPRs provided herein include fusion proteins composed of an extracellular single chain variable fragment (scFv), which binds the target protein with high affinity, that is fused (e.g., via a linker) to the transmembrane and cytoplasmic domains of a phagocytic receptor. For each target protein, CPRs have been identified that selectively bind to the target protein and mediate rapid internalization of the targeted protein.

The cytoplasmic, or intracellular, receptor component of the CPR may comprise any of the TREM2, FCεRIγ (FCERG, or FCER1G), MRC1, MERTK, or CLEC4L receptors; or a hybrid domain composed of multiple domains from these receptors. This disclosure represents the first use of a triggering receptor expressed on myeloid cells-2 (TREM2) domain in a chimeric phagocytic or engulfment receptor.

In the CNS, microglial cells and astrocytes can be phagocytic. The disclosure provides that astrocytes can be transduced with any of the disclosed CPRs in rodents using, e.g., an appropriate gene therapy vector. Further, in the Examples that follow, rodent microglia cells have been transduced efficiently ex vivo with the disclosed CPRs. These engineered microglial may be investigated further ex vivo in relevant models or transplanted into the CNS (e.g., the brain) of subjects in need thereof. Likewise, peripheral monocytes may be engineered ex vivo and transplanted directly into the CNS of the subject.

The disclosed CPRs may be encoded in a nucleic acid vector to enhance delivery and stable transduction of target cells such as astrocytes. Accordingly, the disclosure provides polynucleotides and expression vectors comprising nucleic acids encoding any of the disclosed CPRs. A high-expression vector system under active investigation for its clinical potential is the recombinant adeno-associated viral (rAAV) vector. The disclosure provides that the rAAV vector exhibits efficient transduction in, inter alia, astrocytes, microglial cells, and peripheral monocytes.

Accordingly, in some aspects, the present disclosure provides modified cells comprising a chimeric receptor, wherein the receptor comprises (i) an intracellular phagocytic signaling domain, (ii) an extracellular binding domain comprising a scFv, and (iii) a transmembrane domain positioned between and covalently linked to the intracellular phagocytic signaling domain and the extracellular binding domain. In some embodiments, the modified cell is an astrocyte. In various embodiments, the scFv has affinity for an amyloid beta peptide, a tau protein or α-synuclein protein. In some embodiments, the scFv has specificity (e.g., high specificity) for an amyloid beta peptide, a tau protein or α-synuclein protein. In some embodiments, the scFv has specificity for an amyloid beta peptide. In some embodiments, the scFv has specificity for a tau protein. In some embodiments, the scFv has specificity for an α-synuclein protein. In various embodiments, the modified cells are capable of phagocytosing an amyloid beta peptide, a tau protein or an α-synuclein protein.

In some aspects, provided herein are a plurality of any of the disclosed modified cells. In some embodiments of the plurality, at least 65%, at least 70%, at least 75%, or at least 80% of the plurality are capable of phagocytosing an amyloid beta peptide, a tau protein or an α-synuclein protein.

In some embodiments, the modified cells of the disclosure have been isolated from a subject, such as a mammalian subject, or preferably, a human subject. In some embodiments, the modified cell is a microglial cell. In some embodiments, the modified cell is a peripheral monocyte. In some embodiments, the modified cells are induced pluripotent stem cells (iPSC). The modified cells may comprise microglial cells derived from an iPSC. In certain embodiments, provided herein are iPSC cells containing any of the disclosed vectors encoding a chimeric receptor. In some embodiments, provided herein are iPSC cells containing any of the disclosed vectors encoding a chimeric receptor operably controlled by a promoter active in microglia (e.g., CD68). In some embodiments, these iPSC cells are allowed to differentiate into microglial cells and subsequently administered to a subject. In some aspects, methods of modifying or engineering a cell (e.g., an astrocyte) comprising contacting the cell with any of the disclosed polynucleotides or vectors are provided.

In various aspects of the disclosure, provided herein are chimeric receptors that comprise (i) an intracellular phagocytic signaling domain, (ii) an extracellular binding domain comprising a single-chain variable antibody fragment having affinity for an amyloid beta peptide, tau protein or α-synuclein protein, and (iii) a transmembrane domain positioned between and covalently linked to the intracellular phagocytic signaling domain and the extracellular binding domain. In various embodiments, the scFv comprises an amino acid sequence having at least 90%, at least 92.5%, at least 95%, at least 98%, or at least 99% sequence identity to any of SEQ ID NOs: 1-6. In some embodiments, the scFv comprises any one of SEQ ID NOs: 1-6. In some aspects, provided herein are chimeric receptors that comprise an intracellular phagocytic signaling domain is derived from the TREM2 receptor.

In some embodiments, the transmembrane domain of the chimeric receptors comprises a hinge domain. The transmembrane domain may be covalently linked to the intracellular phagocytic signaling domain and the extracellular binding domain by one or more linkers. In some embodiments, the first intracellular phagocytic signaling domain comprises a TREM2 signaling domain, a MRC1 signaling domain, a MEGF10 signaling domain, a DAP12 signaling domain, a MERTK signaling domain, a FCεRIγ (or FCERG, or FCRG) signaling domain, a M6PR signaling domain, or a CLEC4L signaling domain. In certain embodiments, the first intracellular phagocytic signaling domain comprises a TREM2 signaling domain. In some embodiments, the chimeric receptor comprises an amino acid sequence having at least 85%, at least 90%, at least 92.5%, at least 95%, at least 98%, or at least 99% sequence identity to any of SEQ ID NOs: 26-43. In some embodiments, the chimeric receptors comprise the amino acid sequence of any one of SEQ ID NOs: 26-43. In some aspects, the chimeric receptor comprises a second extracellular binding domain.

In other aspects, provided herein are chimeric receptors that, in addition to a first intracellular phagocytic signaling domain, comprise a second intracellular phagocytic signaling domain that is fused to the first intracellular phagocytic signaling domain. In some embodiments, the chimeric receptors having a second phagocytic signaling domain comprise an scFv that comprises an amino acid sequence having at least 90%, at least 92.5%, at least 95%, at least 98%, or at least 99% sequence identity to any of SEQ ID NOs: 1-6. In some embodiments, the first intracellular phagocytic signaling domain comprises a TREM2 signaling domain, a MRC1 signaling domain, a MEGF10 signaling domain, a DAP12 signaling domain, a MERTK signaling domain, a FCERG signaling domain, a M6PR signaling domain, or a CLEC4L signaling domain; and the second intracellular phagocytic signaling domain comprises a TREM2 signaling domain, a MRC1 signaling domain, a MEGF10 signaling domain, a DAP12 signaling domain, a MERTK signaling domain, a FCERG signaling domain, a M6PR signaling domain, or a CLEC4L signaling domain. In certain embodiments, the first intracellular phagocytic signaling domain comprises a FCERG signaling domain, and the second intracellular phagocytic signaling domain comprises a MRC1 signaling domain. In other embodiments, the first intracellular phagocytic signaling domain comprises a CLEC4L signaling domain.

In certain aspects, the present disclosure provides polynucleotides encoding one or more of the CPRs. In some embodiments, the polynucleotide comprises a sequence encoding a secretion signal peptide, and/or one or more tags (e.g., a FLAG tag). In some embodiments, the signal peptide is selected from a TREM2 signal peptide, a MRC1 signal peptide, a MEGF10 signal peptide, a DAP12 signal peptide, a MERTK signal peptide, a CD8 signal peptide, a M6PR signal peptide, and a DC-SIGN signal peptide.

Further provided are expression vectors (or constructs) comprising any one of these disclosed polynucleotides. These vectors may comprise a heterologous promoter driving expression of the polynucleotide, such as a promoter selected from a CD68 promoter, a Glial Fibrillary Acidic protein (GFAP) promoter, a CMV enhancer/chicken Beta-actin (CAG) promoter, a Microtubule-associated protein 2 (MAP2) promoter or a synapsin 1 (SYN) promoter. In various embodiments, the heterologous promoter is active in astrocyte cells and/or microglial cells.

Also provided herein are recombinant AAV (rAAV) particles comprising such polynucleotides or expression vectors. In some embodiments, these rAAV particles comprise a modified capsid, such as an PHP.eB capsid, an AAV6(Y705F+Y731F+T492V) capsid, or a TM2 capsid. Further disclosed herein are modified cells comprising any of the polynucleotides, vectors or rAAV particles disclosed herein.

In some aspects, the disclosure provides pharmaceutical compositions that comprise a chimeric receptor as described above, or an expression vector as described above, and a pharmaceutically acceptable excipient. Also provided are nanoparticles comprising any of the disclosed chimeric receptors or pharmaceutical compositions.

In other aspects, the disclosure provides methods of treatment of a subject having a disease, disorder or condition (e.g., a neurodegenerative disease, disorder, or condition) comprising administering to the subject one or more CPRs; modified cells expressing on their surface one or more CPRs, as described above (or plurality thereof); pharmaceutical compositions; or rAAV particles or vectors encoding any of the CPRs described above. In some aspects, the subject is a human. The modified cells to be administered in any of the disclosed methods of treatment may be autologous, and/or the method may comprise administering the modified cells into the brain of the subject. In some embodiments, the modified cells, rAAV particles, vectors, or pharmaceutical compositions are administered through an intravenous, intraocular, or intracerebroventricular infusion.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 is a schematic illustrating an exemplary mechanism by which exemplary Chimeric Phagocytic Receptors (CPRs) of the disclosure may be targeted to astrocytes or microglia and tested in relevant models of the AD and PD proteinopathies. This schematic further illustrates the methodology of the experiments discussed in the Examples. A relevant model of AD is the APP mouse, which expresses mutant amyloid precursor protein (APP) (23) (see also Chakrabarty et al., Neuron 85, 519-533, 2015).

FIG. 2 is a schematic illustrating the architectures of exemplary CPRs comprising different intracellular phagocytic domains (ICD). SP, signal peptide; FLAG, flag tag; “scFv Aβ9” and “3A6 scFv” refer to single-chain variable antibody fragments specific to Aβ and tau peptides, respectively; TMD, transmembrane domain; GPI, glycosylphosphatidylinositol anchor; IgK SP, immunoglobulin kappa signal peptide.

FIG. 3 is a schematic illustrating the architectures of additional exemplary CPRs of the disclosure. Single intracellular domain (ICD) receptors and dual intracellular domain (CPR 2) receptors are shown. The ICDs of the dual intracellular domain receptors may comprise any of the disclosed ICDs, except DC-SIGN (CLEC4L). Exemplary CPRs contain a signal peptide (SP), FLAG tag, extracellular single chain variable fragment (scFv), extracellular fragment (i.e., a hinge domain), transmembrane domain (TMD) and intracellular domain (ICD) (bolded). Contiguous segments of exemplary amino acid sequences for each of the exemplary CPR peptides are shown as SEQ ID NOs: 57-62, respectively. The “###” notation in the sequences denotes the position of the tag and scFv domains in the CPR protein represented; and the “***” notation in the sequences denotes breaks in contiguous segments in the CPR protein represented. UniProt ID numbers for each of these peptides are indicated at the right side of the figure.

FIGS. 4A and 4B are images of Western blot gels showing CPR overexpression and membrane anchoring. rAAV vectors encoding CPRs were transiently transfected into HEK 293T cells. The CPRs encoded in the rAAV vectors, all of which were tagged with a FLAG tag (see FIG. 2), contained ICDs derived from the CLEC4L (DC-SIGN), MerTK, FCERG, MRC1, and MEGF10. Cells transfected with CPRs were lysed by N₂ cavitation. In FIG. 4A, whole cell lysate was subjected to electrophoresis in a Western blot. In FIG. 4B, the membrane fraction and supernatant of lysates of HEK cells transfected with rAAV vectors encoding MerTK ICD CPRs were separated by centrifugation at 100,000 g for 1 hour prior to electrophoresis. Western blots were developed using anti-FLAG M2 antibody.

FIG. 5 illustrates the results of a ELISA assay that shows that CPRs containing FCERG ICDs bind their target antigen peptide specifically. A 96-well plate was coated with Aβ42, tau K18, or α-SYN and subsequently coated with a blocking buffer. HEK cells were transfected with rAAV vectors encoding the CPRs. Cells were subsequently lysed by N₂ cavitation. Membrane fraction and supernatant were separated by centrifugation at 100,000 g for 1 hour. The membranes were then solubilized with detergent CHAPSO. ELISAs were then developed with HRP-conjugated anti-FLAG antibody.

FIGS. 6A-6E are fluorescence microscopy images illustrating that expression of full CPRs enhance Aβ, tau and α-SYN uptake into cells engineered to express these CPRs. Several CPR backbones, which lack an extracellular binding domain (EBD) such as a scFv, and CPRs were transiently transfected by rAAV into HEK293 (“HEK”) and monocyte/macrophage-like RAW264.7 cells. Cells depicted in the lower panels of these images were transfected with CPRs tagged with a GFP reporter protein. Images were captured 10 min after incubation with ALEXA FLUOR-555 labelled Aβ42 or tau K18. Overlay of the ALEXA FLUOR-555 and GFP stains are indicated with arrows. FIG. 6A depicts the controls of the experiment, showing that expression of the FCER1G backbone (ICD and TMD) and GPI membrane anchored-Aβ9 scFv did not enhance Aβ42 internalization (or uptake). It further shows that Aβ9 scFv-FCER1G does not enhance tau K18 internalization. FIG. 6B shows that expression of anti-Aβ9 scFv-MRC1 and anti-Aβ9 scFv-FCER1G CPRs increased internalization of Aβ42 into HEK cells. FIG. 6C shows that expression of the anti-tau 3A6 scFv-MRC1 and 3A6 scFv-FCER1G CPRs enhance uptake of tau K18 in HEK cells. FIG. 6D shows that expression of 1D12 scFv-MRC1 and 1D12 scFv-FCER1G CPRs enhanced uptake of the α-SYN target into HEK cells. FIG. 6E shows that expression of anti-Aβ9 scFv CPRs increased uptake of Aβ42 into RAW264.7 cells.

FIGS. 7A-7C are fluorescence microscopy images showing that dual ICD CPR 2 s enhance Aβ and Tau uptake. Full CPR 2 s or CPR 2 backbones were transiently transfected by rAAV into HEK 293T cells. Images were captured 10 minutes after incubation with ALEXA FLUOR-55 (red) labelled Aβ42 or Tau K18. FIG. 7A shows that FCERG-MRC1 and MRC1-FCERG backbones do not enhance Aβ42 or Tau K18 internalization. FIG. 7B shows that Aβ9 scFv-FCERG-MRC1 and 3A6 scFv-FCERG-MRC1 increase uptake of Aβ42 and Tau K18 into HEK cells. FIG. 7C shows that Aβ9 scFv-MRC1-FCERG and 3A6 scFv-MRC1-FCERG increase uptake of Aβ42 and Tau K18 into HEK cells.

FIG. 8 shows delivery of an EGFP reporter transgene in rAAV-PHP.eB particles to adult mice results in widespread transduction of the entire brain. 10¹¹ vector genomes (vgs) of rAAV-PHP.eB-EGFP were delivered intraocularly into both eyes of a 2-month old mouse. Three weeks later, brain tissues were extracted and fixed, and paraffin-embedded tissue was stained with anti-GFP antibody and counterstained with hematoxylin. GFP staining of astrocytes was observed throughout the brain. Image magnification is 500 mm and 100 mm in the upper and lower images, respectively.

FIGS. 9A and 9B are fluorescence microscopy images that show microglia transduction in brain slice cultures. FIG. 9A shows that rAAV particles containing a triple mutant capsid 6 (TM6) and a vector controlled by a CD68 promoter deliver EGFP selectively to microglial cells in organotypic brain slice cultures (BSCs) at 0 days in vitro (DIV). Slices are fixed and stained at 14 DIV. Transduction is confirmed by co-staining with anti-CD68 antibody. Cells were also stained with DAPI (indicated with diamond arrows). Overlay of the two antibodies was observed (as indicated by arrows). FIG. 9B shows that BSCs prepared from CX3CR1-EGFP knock-in mice (see Garcia et al., Methods Mol Biol. 2013; 1041:307-17, incorporated herein by reference) contain microglial cells stained green. Transduction with AAV2/TM6-CD68-mCherry (red) shows expression in microglial cells (red). An overlay of images shows microglial specificity (indicated by arrows) and 40% transduction efficiency.

FIG. 10 shows the results of an in vitro assay that evaluated quantification of A139 uptake by A39-specific CPRs in human cells. CPRs comprising an scFv A139 and an intracellular receptor selected from DAP12, MRC1, and FCERG were evaluated. Total fluorescently tagged-Aβ in post-incubation media and HEK293 cell lysate were measured by ELISA. Aβ levels in cells treated with control plasmid encoding scFv A139 under the control of a CD8 promoter (which is not phagocytic) were set as 100%. DAP12, MRC1, and FCERG backbones (“BB”), which are not phagocytic, also served as controls.

FIG. 11 shows the transduction of astrocytes with rAAV-FCERG CPRs having AAV2 triple mutant (AAV-TM2) capsids under control of the GFAP promoter. Transduction efficiency was verified by RFP fluorescence (left) and Western blot of CPRs with anti-FLAG antibody (right).

FIGS. 12A and 12B show the uptake and clearance of Aβ42 and Tau protein by rAAV-CPR-transduced astrocytes. FIG. 12A shows the time-dependent uptake and clearance of Aβ42 by Aβ9-FCERG-transduced astrocytes. The first row shows that non-specific FCERG CPR (or the backbone, “BB”) did not uptake Aβ42 until 48 hours of incubation. The second row shows Aβ9-FCERG enhanced uptake of Aβ42 significantly by the 12 hour time point. In third row, the media was replaced with fresh Aβ42-free media at 24 hours. Aβ42 fluorescence deceased dramatically over 12 hours. FIG. 12B shows the time-dependent uptake and clearance of tau K18 protein by 3A6-FCERG. The uptakes of tau K18 by 3A6-FCERG is faster than the process of Aβ42 by AB9-FCERG. Here, Tau K18 was incubated with 3A6-FCERG-transduced astrocytes for 3 hours and then replaced media with fresh tau-free media. Fluorescence decreased significantly over 24 hours.

FIGS. 13A and 13B show the results of an in vivo assay that evaluated quantification of Aβ9 uptake by Aβ9-specific CPRs in CRND8 mice. CPRs comprising an scFv Aβ9 (“scFv9”) and an intracellular receptor selected from MRC1 and FCERG were evaluated. rAAV-CPRs were administered stereotaxically to 8-month-old CRND8 mice. Two months later, the brains were harvested for biochemical and immunohistochemical analysis. One hemibrain was fixed and processed to paraffin blocks. Consecutive sections were stained with anti-FLAG for expression, pan-Abeta antibody for amyloid aggregation, GFAP and IBA 1. The amyloid burden was quantified using ImageScope analysis in 3 non-consecutive sections per mouse. n=6-8, **p<0.01, Scale bars 150 mM.

### DEFINITIONS

As used in the specification and claims, the singular term “a”, “an” and “the” include plural references unless the context clearly dictates otherwise. For example, the term “a cell” includes a plurality of cells, including mixtures thereof.

The terms “ameliorate” and “treat” are used interchangeably and include both therapeutic and prophylactic treatment. Both terms mean decrease, suppress, attenuate, diminish, arrest, or stabilize the development or progression of a disease (e.g., a neurodegenerative disease or disorder described herein).

The term “administration” or “administering” includes routes of introducing the compound of the invention(s) to a subject to perform their intended function. Examples of routes of administration that may be used include injection (subcutaneous, intravenous, parenteral, intraperitoneal, intrathecal, ICV, intraocular), stereotaxic surgery; and oral, inhalation, rectal and transdermal routes of administration. The pharmaceutical preparations may be given by forms suitable for each administration route. For example, these preparations are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. The rAAV particles, modified cells, nanoparticles, vectors and compositions of the disclosure can be administered alone, or in conjunction with either another agent as described above or with a pharmaceutically-acceptable carrier, or both. They may be administered prior to the administration of the other agent, simultaneously with the agent, or after the administration of the agent.

The term “agent” refers to a small molecule compound, a polypeptide, polynucleotide, or fragment, or analog thereof, or other biologically active molecule.

As used herein, an “amyloid-related disease or disorder” includes Alzheimer's Disease (AD) and Parkinson's Disease (PD). Exemplary diseases, disorders or conditions of the disclosure include, but are not limited to, AD, Parkinson's Disease, Amyotrophic Lateral Sclerosis (“ALS”), Multiple Sclerosis (“MS”), Stroke and Frontal temporal Dementia.

An “ectodomain” or “extracellular domain” refers to the domain of a transmembrane protein that extends into the extracellular space.

An “antibody” refers to an immunoglobulin molecule capable of specific or selective binding to a target, such as a carbohydrate, polynucleotide, lipid, polypeptide, etc., through at least one antigen recognition site, located in the variable region of the immunoglobulin molecule. As used herein, the term “antibody” encompasses not only intact (e.g., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity or selectivity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

The term “antigen-binding fragment” of an antibody (or simply “antibody fragment”), as used herein, refers to one or more fragments of an antibody that retain the ability to specifically bind or selectively bind to an antigen. It has been shown that the antigen-binding function of an antibody can be performed by fragments of a full-length antibody. Such antibody embodiments may also be bispecific, dual specific, or multi-specific formats; specifically binding to two or more different antigens. Multispecific and bispecific antibody constructs are well known in the art and described and characterized in Kontermann (ed.), Bispecific Antibodies, Springer, NY (2011), and Spiess et al., Mol. Immunol. 67(2):96-106 (2015), each of which are incorporated by reference herein.

Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (see Ward et al., (1989) Nature 341:544-546 and Winter et al., PCT Publication No. WO 1990/05144, herein incorporated by reference), which comprises a single chain variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5). Variable regions can be linked to encode single chain Fv regions. Multiple Fv regions can be linked to confer binding ability to more than one target or chimeric heavy and light chain combinations can be employed. Any appropriate method may be used for cloning of antibody variable regions and generation of recombinant antibodies.

The term “bispecific,” as used herein, signifies having two different antigen binding domains, each domain being directed against a different ligand or epitope.

As used herein, the terms “directed against”, “selectively binds to,” oand “specifically binds to” a ligand or an epitope are well understood in the art. A molecule is said to exhibit “specific binding” or “selective binding” if it reacts more frequently, more rapidly, with greater duration and/or with greater affinity with a particular target ligand or epitope than it does with alternative targets. An antibody fragment or heterologous peptide “specifically binds” to a target ligand or epitope if it binds with greater affinity, avidity, more readily, and/or with greater duration than it binds to other substances. For example, a single-chain antibody fragment (scFv) that specifically (or preferentially) binds to a ligand or epitope therein is a fragment that binds this target antigen with greater affinity, avidity, more readily, and/or with greater duration than it binds to other ligands or other epitopes. It is also understood with this definition that, for example, a heterologous peptide or scFv that specifically binds to a first target ligand or antigen may or may not specifically or preferentially bind to a second target ligand or antigen. As such, “specific binding” or “preferential binding” does not necessarily require (although it may include) exclusive binding.

“Disease” means any condition or disorder that damages or interferes with the normal function of a cell, tissue, organ or organism. Exemplary diseases of the disclosure are neurodegenerative diseases.

The term “epitope” includes any polypeptide capable of specific binding to an antibody or fragment thereof (e.g., an scFv). In certain embodiments, epitope determinants include chemically active surface groupings of molecules such as amino acids, sugar side chains, phosphoryl, or sulfonyl, and, in certain embodiments, may have specific three-dimensional structural characteristics, and/or specific charge characteristics. An epitope is a region of an antigen that is bound by an antibody or fragment thereof (or an scFv). In certain embodiments, an antibody is said to specifically or selectively bind to an antigen when it preferentially recognizes its target antigen in a complex mixture of proteins and/or macromolecules.

The term “ligand” includes any polypeptide capable of specific or selective binding to a receptor or fragment thereof (e.g. a transmembrane receptor). In certain embodiments, ligands include chemically active groupings of molecules such as amino acids, sugar side chains, phosphoryl, or sulfonyl, and, in certain embodiments, may have specific three-dimensional structural characteristics, and/or specific charge characteristics.

The term “effective amount” includes an amount effective, at dosages and for periods of time necessary, to achieve the desired result, e.g., sufficient to treat a disease or disorder delineated herein. An effective amount of pharmaceutical compositions and rAAV particles of the disclosure may vary according to factors such as the disease state, age, and weight of the subject, and the ability to elicit a desired response in a cell or in the subject. Dosage regimens may be adjusted to provide the optimum therapeutic response. An effective amount is also one in which any toxic or detrimental effects (e.g., side effects) are outweighed by the therapeutically beneficial effects.

The term “subject” includes organisms which are capable of suffering from a disorder as described herein or who could otherwise benefit from the administration of a compound of the present disclosure, such as human and non-human animals. Preferred humans include human patients suffering from or prone to suffering from diseases or disorders as discussed above, as described herein. Mammalian species that may benefit from the disclosed methods of treatment include, but are not limited to, apes; chimpanzees; orangutans; humans; monkeys; domesticated animals such as dogs and cats; livestock such as horses, cattle, pigs, sheep, goats, and chickens; and other animals such as mice, rats, guinea pigs, and hamsters. A “subject in need of treatment” includes a subject diagnosed, e.g., by a medical or veterinary professional, as suffering from or susceptible to a neurodegenerative disease, disorder or condition described herein.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable excipient” means any non-toxic excipient that, upon administration to a recipient, is capable of carrying, either directly or indirectly, any of the rAAV particles, nanoparticles, vectors, or modified cells of the disclosure.

As used herein, the term “phagocytosis” refers to the process in which a cell engulfs or ingests material, such as other cells or parts of cells (e.g., bacteria), particles, or dead or dying cells. A cell that capable of performing this function is called a phagocyte. Immune phagocytes include neutrophils, monocytes, macrophages, mast cells, B cells, eosinophils, and dendritic cells.

As used herein, the term “chimeric phagocytic receptor” refers to a fusion protein consisting of multiple domains, at least one of which is a phagocytic signaling domain. This signaling domain activates in a cell expressing the chimeric phagocytic receptor (CPR) the ability to engulf a protein, microbe, or cell expressing an antigen with which a binding domain of the CPR interacts. CPRs typically contain an extracellular binding domain, transmembrane domain, and an intracellular phagocytic signaling domain. CPRs are also referred to in the art as chimeric engulfment receptors and chimeric endocytic receptors.

As used herein, the term “CPR 2” refers to a chimeric phagocytic receptor (or a chimeric engulfment receptor) containing two intracellular domains, i.e., a first and a second intracellular phagocytic domain, or a dual ICD. The dual ICDs of the disclosure may comprise any of the disclosed ICDs. In exemplary embodiments, the dual ICDs of the CPRs of the disclosure comprise an FCERG ICD and an MRC1 ICD.

As used herein, the terms “monocyte” and “peripheral monocyte” (also referred to as a peripheral blood mononuclear cell) refer to a type of leukocyte, or white blood cell, that can engulf and digest cellular debris, foreign substances, microbes, cancer cells, and anything else that does not have the types of cell surface markers specific to healthy body cells in a process called phagocytosis. Monocytes are found in essentially all tissues, where they patrol for potential pathogens by amoeboid movement. They take various forms (with various names) throughout the body (e.g., histiocytes, Kupffer cells, alveolar macrophages, microglia, and others), but all are part of the mononuclear phagocyte system. Besides phagocytosis, they play a critical role in non-specific defense (innate immunity) and also help initiate specific defense mechanisms (adaptive immunity) by recruiting other immune cells such as lymphocytes. For example, they are important as antigen presenters to T cells. Beyond increasing inflammation and stimulating the immune system, monocytes also play an important anti-inflammatory role and can decrease immune reactions through the release of cytokines. Monocytes are the largest type of leukocyte and can differentiate into macrophages and myeloid lineage dendritic cells. As a part of the vertebrate innate immune system, monocytes also influence the process of adaptive immunity. There are at least three subclasses of monocytes in human blood based on their phenotypic receptors: classical monocyte or CD14++CD16− monocyte (characterized by high level expression of the CD14 cell surface receptor), non-classical monocyte or CD14+CD16++ monocyte (shows low level expression of CD14 and additional co-expression of the CD16 receptor), and intermediate monocyte or CD14++CD16+ monocyte (characterized by high level expression of CD14 and low level expression of CD16).

As used herein, the term “dendritic cell” (DC) refers to a type of antigen-presenting cell of the mammalian immune system. Their main function is to process antigen material and present it on the cell surface to the T cells of the immune system. Dendritic cells bridge the two arms of the immune system by acting as messengers between the innate and the adaptive immune systems. Dendritic cells are present in those tissues that are in contact with the external environment, such as the skin (where there is a specialized dendritic cell type called the Langerhans cell) and the inner lining of the nose, lungs, stomach and intestines. They can also be found in an immature state in the blood. Once activated, they migrate to the lymph nodes where they interact with T cells and B cells to initiate and shape the adaptive immune response. At certain development stages they grow branched projections or dendrites that give the cell its name.

As used herein, the term “antigen presenting cell” (APC) refers to a cell that displays antigen complexed with major histocompatibility complexes (MHCs) on its surfaces, a process known as antigen presentation. T cells may recognize these complexes using their T cell receptors. Antigen presenting cells are found in a variety of tissue types. Professional antigen presenting cells, including macrophages, B cells, and dendritic cells, present foreign antigens to helper T cells, while other cell types can present antigens originating inside the cell to cytotoxic T cells. In addition to the MHC family of proteins, antigen presentation relies on other specialized signaling molecules on the surfaces of both APCs and T cells. Antigen presenting cells are vital for effective adaptive immune response, as the functioning of both cytotoxic and helper T cells is dependent on APCs. Antigen presentation allows for specificity of adaptive immunity and can contribute to immune responses against both intracellular and extracellular pathogens. It is also involved in defense against tumors.

The term “effective amount,” as used herein, refers to an amount that is capable of treating or ameliorating a disease or condition or otherwise capable of producing an intended therapeutic effect.

As used herein, the terms “engineered cell” and “modified cell” are intended to refer to a cell into which an exogenous polynucleotide segment (such as DNA segment that leads to the transcription of a biologically active molecule) has been introduced. Therefore, engineered cells are distinguishable from naturally occurring cells, which do not contain a recombinantly introduced exogenous DNA segment. Engineered cells are, therefore, cells that comprise at least one or more heterologous nucleic acid segments introduced through the hand of man.

As used herein, the term “expression” may refer to the expression of a receptor on a cell surface and/or the expression of a protein in the cell (i.e., expression of a protein encoded on a nucleic acid in the cell). A chimeric phagocytic receptor may be expressed from a nucleic acid in the cell and subsequently trafficked to the cell membrane for expression on the cell surface.

The term “promoter,” as used herein refers to a region or regions of a nucleic acid sequence that regulates transcription.

The term “regulatory element,” as used herein, refers to a region or regions of a nucleic acid sequence that regulates transcription. Exemplary regulatory elements include, but are not limited to, enhancers, post-transcriptional elements, transcriptional control sequences, and such like.

The term “operably linked,” as used herein, refers to the nucleic acid sequences being linked are typically contiguous, or substantially contiguous, and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

As used herein, the term “variant” refers to a molecule (e.g. a chimeric receptor protein sequence) having characteristics that deviate from what occurs in nature, e.g., a “variant” is at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type protein. Variants of a protein molecule, e.g. a chimeric receptor, may contain modifications to the amino acid sequence (e.g., having 1, 2, 3, 4, 5, 6, 7, 8, 9, 10, 10-15, or 15-20 amino acid substitutions) relative to the wild type protein sequence, which arise from point mutations installed into the nucleic acid sequence encoding the protein. These modifications include chemical modifications as well as truncations, such as truncations at the N- or C-terminus of a protein sequence.

“Percent (%) identity” refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y. Generally, computer programs are employed for such calculations. Exemplary programs that compare and align pairs of sequences, include ALIGN (Myers and Miller, 1988), FASTA (Pearson and Lipman, 1988; Pearson, 1990) and gapped BLAST (Altschul et al., 1997), BLASTP, BLASTN, or GCG (Devereux et al., 1984).

Typically, the selected sequence and the reference sequence will have at least about 76, 77, 78, 79, 80, 81, 82, 83, 84 or even 85 percent sequence identity, and more preferably, at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared. The percentage of sequence identity may be calculated over the entire length of the sequences to be compared or may be calculated by excluding small deletions or additions which total less than about 25 percent or so of the chosen reference sequence. The reference sequence may be a subset of a larger sequence.

When highly-homologous fragments are desired, the extent of percent identity between the two sequences will be at least about 80%, preferably at least about 85%, and more preferably about 90% or 95% or higher, as readily determined by one or more of the sequence comparison algorithms well-known to those of skill in the art, such as e.g., the FASTA program analysis described by Pearson and Lipman (1988) and blastn computer program based on the algorithm of Brutlag et al. (Comp. App. Biosci. 6:237-245 (1990). A preferred method for determining the best overall match between a query sequence (e.g., a sequence of the present disclosure) and a subject sequence, also referred to as a global sequence alignment, can be determined using the FASTA or blastn. In a sequence alignment the query and subject sequences are either both nucleotide sequences or both amino acid sequences. The result of said global sequence alignment is expressed as percent identity. Preferred parameters used in a FASTA amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. Whether a nucleotide is matched/aligned is determined by results of the FASTA sequence alignment. This percentage is then subtracted from the percent identity, calculated by the above FASTA program using the specified parameters, to arrive at a final percent identity score. This final percent identity score is what is used for the purposes of the present disclosure.

### DETAILED DESCRIPTION OF THE INVENTION

Aspects of this disclosure relate to novel complex chimeric phagocytic receptors and polynucleotides and vectors encoding these receptors, for use in treating neurodegenerative diseases. These complex CPRs have binding affinity to plaque-forming protein ligands in the CNS and mediate the clearance of these protein aggregates from the CNS through phagocytic mechanisms. Exemplary CPRs of this disclosure include CPRs having an intracellular phagocytic signaling domain derived from the triggering receptor expressed on myeloid cells-2 (TREM2) receptor and the mannose 6 phosphate receptor (M6PR). Additional CPRs of this disclosure comprise more than one intracellular phagocytic signaling domain, such as two intracellular phagocytic signaling domains.

Further aspects of this disclosure relate to modified or engineered cells that express these CPRs on the cell surface. These modified cells may have increased (or enhanced) phagocytic activity against one or more protein aggregate ligands by virtue of expression of any of the disclosed CPRs. Administration of one or more, or a plurality of these modified cells, to a subject may provide treatment of a neurodegenerative disease such as Alzheimer's Disease or Parkinson's Disease. Administration of one or more, or a plurality of these modified cells, to a subject may result in improvement of symptoms associated with AD or PD pathologies in the subject. Accordingly, provided herein are pharmaceutical compositions comprising these modified cells, and methods of treatment comprising a step of administering these cells to a subject, such as a mammalian subject (e.g., a human).

The disclosed CPRs are analogous to chimeric antigen receptor T-cell (CAR-T) studies (18), but instead of engineering T-cells to target a specific protein, they harness phagocytic cells to target either Aβ or tau. Like CAR-Ts, appropriately targeted CPRs may have applicability to disorders other than neurodegenerative disorders. Although others have purported to report the development of CPRs as potential cancer therapeutics (see Morrissey et al. elife. 2018; 7:e36688, incorporated herein by reference), no in vivo effects of potential therapeutic efficacy in a neurodegenerative setting have been reported. The Morrissey group developed CPRs that activated the engulfment of whole cancer cells, whereas the disclosed CPRs activate engulfment of proteins.

In some aspects, the expression on the surface of any of the disclosed CPRs activate enhanced phagocytic activity in a cell (e.g., a glial cell or a monocyte). In some embodiments, wild-type or modified cells (e.g., human cells) that lacked significant phagocytic activity are engineered to exhibit phagocytic activity against a protein aggregate, such as tau, alpha-synuclein or amyloid-beta, by virtue of expression of any of the disclosed CPRs. In some embodiments, wild-type cells that exhibit phagocytic activity are engineered to exhibit increased phagocytic activity against a protein aggregate by virtue of expression of any of the disclosed CPRs. These modified cells may be engineered by contacting the cell ex vivo with a polynucleotide or vector encoding the CPR. These modified cells may be isolated from a mammalian subject. These modified cells may be engineered for delivery back into the same subject from which they were isolated, in an autologous delivery. In other embodiments, these modified cells may be engineered for delivery into a subject different from the subject from which they were isolated, in an allogeneic delivery.

As demonstrated in the Examples of the disclosure, the modified cells provided herein exhibit activity against protein aggregates in the brain, such as amyloid-beta aggregates. In various embodiments, the modified cells are capable of phagocytosing an amyloid beta peptide, a tau protein or an α-synuclein protein. In some aspects, provided herein are a plurality of any of the disclosed modified cells. In some embodiments of the plurality, at least 65%, at least 70%, at least 75%, at least 80%, or more than 80% of the plurality are capable of phagocytosing an amyloid beta peptide, a tau protein or an α-synuclein protein.

The methods of treatment exemplified in this disclosure may comprise engineering a cell to express any of the disclosed CPRs by contacting a mammalian cell ex vivo with a polynucleotide or expression vector encoding, and administering to the CNS (e.g., infused to the brain) of a mammalian subject the engineered cell. In some embodiments, the engineered cell is isolated from the brain of the mammal. The mammal may be a human. Alternatively, the mammal may be a rodent, such as a neonatal or adult mouse. Accordingly, in some embodiments, the cell has been isolated from a mammalian subject, such as a human subject. In some embodiments, the modified cell is an astrocyte. Astrocytes are star-shaped (macro)glial cells in the CNS that express markers such as glial fibrillary-associated protein (GFAP), N-Myc downstream-regulated gene 2 (NDRG2), and vimentin (Rosario et al.).

Astrocytes help form the physical structure of the brain and provide maintenance of the blood-brain barrier. Astrocytes are resistant to transduction by AAV and other viral vectors. As demonstrated in the Examples, mammalian astrocytes are efficiently and selectively transduced with the disclosed novel viral vectors. For instance, intracerebroventricular (ICV) or intravenous (IV) delivery to mammalian subjects of one or more the disclosed rAAV vectors provides effective transduction of astrocytes, and reduces time and cost compared to stereotaxic (stereotactic) surgery.

Provided herein are modified cells comprising any of the disclosed polynucleotides, expression vectors, rAAV particles, or pharmaceutical compositions. The modified cell may be an astrocyte. In some embodiments, the modified cell is a microglial cell. Microglia are glial cells in the CNS that express markers such as ionized calcium-binding adapter molecule 1(Iba-1) (Rosario et al.), TMEM119, P2Y₁₂ and HEXB. Microglia provide overall brain maintenance and able to phagocytose infectious material and foreign materials, and may display the resulting antigens for T-cell activation. In some embodiments, the cell is a monocyte-like (or macrophage-like) RAW264.7 cell. In some embodiments, the cell is an HEK293 cell, an H4 (human neuroglioma) cell, or a CHO cell. In some embodiments, the cell is a human stem cell, such as a human pluripotent stem cell. In some embodiments, the cell is an induced pluripotent stem cell (iPSC). In particular embodiments, the cell is an iPSC that can be (or has been) differentiated into a microglial cell or an astrocyte cell. In some embodiments, the methods of treatment comprise the administration (e.g., into the brain) of iPSCs derived from the subject that are engineered to express any of the disclosed CPRs. In some embodiments, the methods of treatment comprise the administration (e.g., into the brain) of two groups of iPSCs derived from the subject—undifferentiated iPSCs and iPSCs that have differentiated into a glial cell such as a microglial cell. These two groups may be administered in tandem, or at separate times or intervals. In exemplary embodiments, the disclosed methods of treatment comprise intraocular, ICV, or IV administrations of modified cells, pharmaceutical compositions, or rAAV particles or expression vectors, into the CNS of the subject. In particular embodiments, the methods comprise administering any of the disclosed the rAAV particle intravenously, intraocularly, or intracerebroventricularly into the subject's brain. This step of administering is performed in vivo in the subject,

Aspects of this disclosure relate to the delivery of CPRs and/or modified cell expressing a CPR to glial cells of the CNS. Likewise, aspects of this disclosure relate to the engineering of modified glial cells for delivery to a subject suffering from a neurodegenerative disease. In particular embodiments, the disclosure provides methods of preparing modified astrocytes that express any of the disclosed CPRs. Astrocytes are macroglial cells in the CNS that help provide physical structure and metabolic support to neurons in the brain. In some embodiments, the disclosure provides improved methods of delivery of these CPRs to astrocytes, including the delivery of expression vectors encoding CPRs to glial cells such as astrocytes using a recombinant AAV technique.

Accordingly, provided herein are rAAV particles comprising any of the disclosed vectors, for instance a vector containing an GFAP promoter, and further comprising a PHP.eB capsid (see FIG. 8). Further provided herein are rAAV particles comprising any of the disclosed vectors, for instance a vector containing a CD8 promoter, and further comprising a TM6 capsid. These rAAV particles may be designed for delivery to astrocytes, monocytes, or microglia, e.g., in modified cell or in a pharmaceutical composition or nanoparticle. In some embodiments, rAAV particles containing a GFAP promoter are delivered to astrocytes. Exemplary such particles may be referred to as “rAAV-PHP.eB-GFAP-CPR.” In some embodiments, rAAV particles containing a CD8 promoter are delivered to microglia and/or monocytes. Exemplary such particles may be referred to as “rAAV-TM6-CD8-CPR,” or simply “rAAV-TM6.”

Further provided herein are rAAV particles comprising any of the disclosed vectors, for instance a vector containing a CD8 promoter, and further comprising an AAV triple mutant (TM2) capsid. These rAAV particles may be designed for delivery to astrocytes, monocytes, or microglia, e.g., in modified cell or in a pharmaceutical composition or nanoparticle. In some embodiments, rAAV particles containing a GFAP promoter are delivered to astrocytes. In some embodiments, rAAV particles containing a CD8 promoter are delivered to microglia and/or monocytes. Exemplary such particles may be referred to as “rAAV-TM2-CD8-CPR” or simply “rAAV-TM2.”

In certain embodiments, the neurodegenerative disease, disorder or condition that is targeted for treatment by the disclosed methods and compositions is implicated by the activity, localization or binding behavior of a protein aggregate. The extracellular binding domain of the disclosed CPRs may comprise a single-chain antibody binding fragment (scFv) that has affinity for one or more of these protein aggregates. Exemplary scFv's that the extracellular binding domains of the disclosed CPRs comprise include, but are not limited to, scFv's against amyloid beta (Aβ), tau, and alpha-synuclein (α-SYN) peptides. The CPRs provided herein may comprise scFv's targeting other proteins or antigens. The scFv's of the disclosed CPRs may be generated de novo. In other embodiments, the scFv's of the disclosed CPRs may be generated or derived from an experimental antibody or a commercially available antibody therapeutic, such as a therapeutic approved by a regulatory authority such as the FDA or EMA. The scFv's may be derived from monoclonal or polyclonal antibodies. Accordingly, in some aspects, the present disclosure provides modified cells comprising a chimeric receptor, wherein the receptor comprises (i) an intracellular phagocytic signaling domain, (ii) an extracellular binding domain comprising a scFv, and (iii) a transmembrane domain positioned between and covalently linked to the intracellular phagocytic signaling domain and the extracellular binding domain. In some embodiments, the modified cell is an astrocyte.

In some embodiments, the scFv has specificity for a target protein comprising an amyloid beta peptide. In some embodiments, the scFv has specificity for a tau protein. In some embodiments, the scFv has specificity for an α-synuclein protein. The choice of the particular scFv for incorporation into a CPR depends on the protein associated with the disease or disorder that the CPR is designed to treat. In some embodiments, the binding of a target protein to the scFv of the CPR may induce certain biological pathways or identify the target protein for elimination or clearance.

Exemplary CPR extracellular binding domain scFv's against amyloid beta include anti-beta peptide 9 (Aβ9), anti-beta peptide 42 (Aβ42), anti-beta peptide 2.1.3 (Aβ2.1.3), and anti-pan beta peptide (pan-Aβ) scFv's, such as B 11. Pan-Amyloid beta antigen binding fragments are said to recognize multiple (up to all) types of human amyloid beta peptide antigens (see, e.g., Levites et al., J. Neuroscience. 26(46):11923-11928 (2006), incorporated herein by reference). Exemplary extracellular domains against tau include anti-tau 3A6 scFv, anti-tau PH2 scFv, and the pan-tau antibody Tau5 (see Croft C L, et al. PLoS ONE, 13(4):e0195211 (2018) and Goodwin et al. Molecular Therapy 29(2), February 2021, each of which is incorporated herein by reference. Additional exemplary extracellular domains against tau include anti-tau K18 scFv, and scFv's against mutant tau proteins having pro-aggregant mutations S320F, P301L, and/or A152T (see Croft et al., J. Exp. Med. 216(3):539-55 (2019), incorporated herein by reference). Tau K18 is a human tau fragment (corresponding to residues 244±372 relative to 2N₄R human tau with an ATG codon added at the N-terminus (see Croft C L, et al. PLoS ONE, 13(4):e0195211 (2018), incorporated herein by reference).

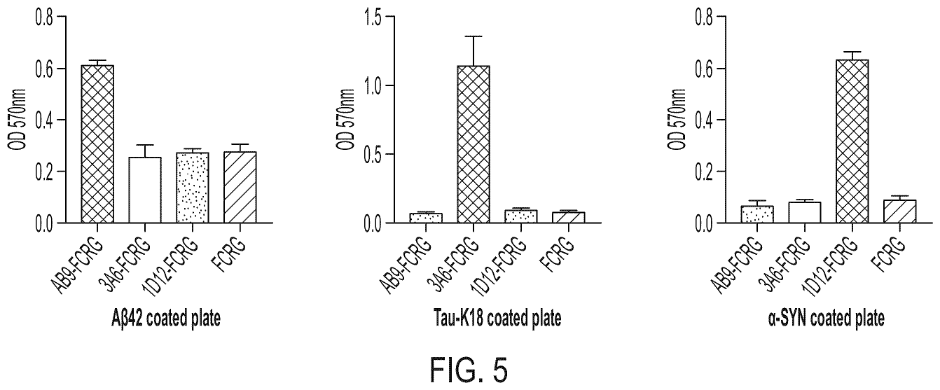

The binding affinities of the binding domain scFv's of the disclosure in vitro can be determined by a variety of methods including equilibrium dialysis, equilibrium binding, gel filtration, ELISA, surface plasmon resonance, or spectroscopy (e.g., using a fluorescence or FACS assay). In some embodiments, an ELISA assay may be used to show that CPRs (e.g., CPRs containing FCERG ICDs) bind their target antigen peptide specifically (see FIG. 5). Binding affinities in vivo can be inferred, e.g., by activity in a functional in vivo assay such as a cell metabolic activity assay or end-product detection assay such as an amyloid plaque detection assay; or directly by methods including in vivo imaging in a subject, e.g., immunohistochemistry (IHC), bioluminescence imaging (BLI), Magnetic Resonance Imaging (MRI), positron emission tomography (PET), electron microscopy, X-ray computed tomography, Raman imaging, optical coherence tomography, absorption imaging, thermal imaging, fluorescence reflectance imaging, fluorescence microscopy, fluorescence molecular tomographic imaging, nuclear magnetic resonance imaging, X-ray imaging, ultrasound imaging, photoacoustic imaging, lab assays, or in any situation where tagging, staining, or imaging is required.

The disclosed CPRs may contain an intracellular phagocytic signaling domain (ICD). The ICD may be derived from a receptor in the Immunoglobulin (Ig) superfamily or the C-type lectin superfamily. In some embodiments, the ICD is derived from a type II transmembrane protein (such as CLEC4L) or a type I transmembrane protein. The disclosed CPRs may contain an ICD selected from a TREM2 signaling domain, a macrophage mannose receptor 1 (MRC1) signaling domain, a multiple epidermal growth factor-like domains protein 10 (MEGF10) signaling domain, a DAP12 signaling domain, a tyrosine-protein kinase Mer (MerTK) signaling domain, a high-affinity immunoglobulin epsilon receptor subunit gamma (FCεRIγ (or FCER1G, or FCERG)) signaling domain, a mannose 6 phosphate receptor (M6PR) signaling domain, or a C-type lectin domain family 4 member L (CLEC4L) signaling domain, or a hybrid of two or more of these domains. (DAP12 is a 12 kDa transmembrane protein that is a key signal transduction receptor element in Natural Killer T cells.) The chimeric phagocytic receptors may comprise ICDs derived from receptors derived from mouse or humans. In exemplary embodiments, the CPRs comprise ICDs derived from human receptors.

The ICD may be any portion of a phagocytic signaling molecule that retains sufficient signaling activity. In some embodiments, a full-length or full-length intracellular component of a phagocytic signaling molecule is used. In some embodiments, a truncated portion of a phagocytic signaling molecule or intracellular component of a phagocytic signaling molecule is used, provided that the truncated portion retains sufficient signal transduction activity. In further embodiments, an ICD is a variant of an entire or truncated portion of a phagocytic signaling molecule, provided that the variant retains sufficient signal transduction activity (i.e., is a functional variant).

In some embodiment, the ICD of the CPR includes any portion of one or more co-stimulatory molecules, such as a signaling domain from CD3, Fc epsilon RI gamma chain, or any derivative or variant thereof. These portions may be referred to herein as a “co-stimulatory domain.”

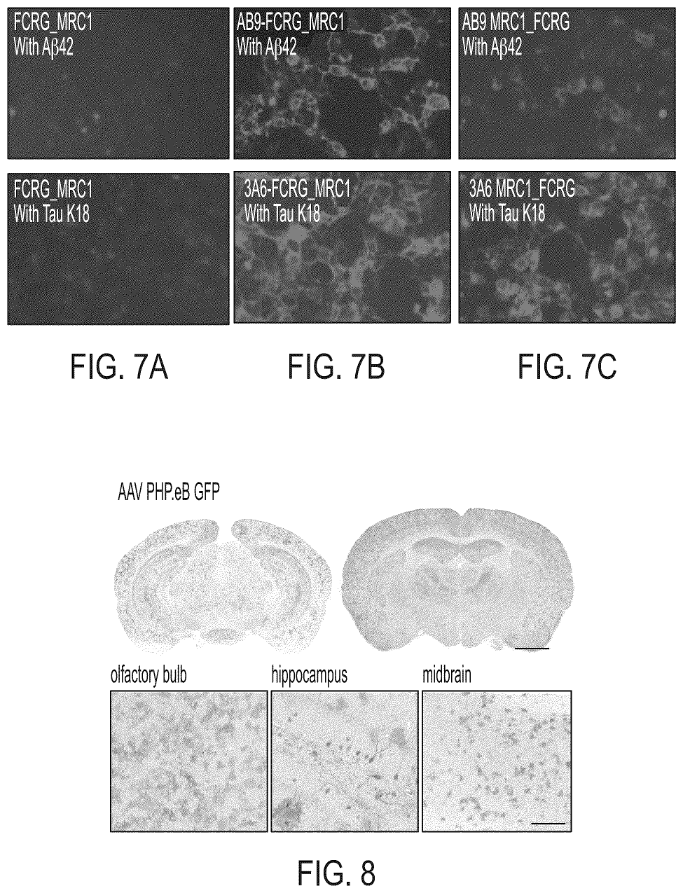

In some embodiments, the CPR may contain a first ICD and a second ICD (i.e., is a dual ICD CPR, or a CPR 2). In some embodiments, the first ICD and the second ICD comprise an FCERG ICD and an MRC1 ICD, respectively. (See FIGS. 7A-7C.) In other embodiments, the first ICD and the second ICD comprise an MRC1 ICD and an FCERG ICD, respectively. In some embodiments, the first ICD and/or the second ICD comprises a TREM2 ICD or a M6PR ICD. In some embodiments, the first ICD is a TREM2 ICD.

The CPRs of the present disclosure may comprise a transmembrane domain that connects and is positioned between the extracellular domain and the engulfment signaling domain. The transmembrane domain is a hydrophobic alpha helix that transverses the host cell membrane and anchors the CPR in the host cell membrane. The transmembrane domain may be directly fused to an ICD or fused to a linker domain that is itself fused to an ICD. In some embodiments, the transmembrane domain may be directly fused to the extracellular binding domain (EBD) or fused to a linker domain that is itself fused to the EBD. In certain embodiments, the transmembrane domain is derived from an integral membrane protein (e.g., a receptor, cluster of differentiation (CD) molecule, enzyme, transporter, or cell adhesion molecule). The transmembrane domain can be selected from the same molecule as the EBD or the ICD. In certain embodiments, the transmembrane domain and the EBD are each selected from different molecules. In other embodiments, the transmembrane domain and the ICD are each selected from different molecules. In still other embodiments, the transmembrane domain, the EBD, and the ICD are each selected from different molecules.

The transmembrane domain (TMD) of the disclosed CPRs may be selected from a TREM2 transmembrane domain, a MRC1 transmembrane domain, a MEGF10 transmembrane domain, a DAP12 transmembrane domain, a MerTK transmembrane domain, a CD8 (or CD8a) transmembrane domain, a M6PR transmembrane domain, or a CLEC4L transmembrane domain. In some embodiments, the transmembrane domain of the chimeric receptors comprises a hinge domain or region. In some embodiments, the hinge region is a CD8 hinge region. The transmembrane domain may be covalently linked to the intracellular phagocytic signaling domain and the extracellular binding domain by one or more linkers. In some embodiments, the TMD is covalently fused to the ICD and/or the EBD. In some embodiments, the TMD is fused to the ICD and covalently linked to the EBD by one or more linkers. In some embodiments, the TMD is fused to the EBD and covalently linked to the ICD by one or more linkers. The transmembrane domain may be derived from a natural source or from a synthetic source.

As used herein, a “CPR backbone” describes a fusion or chimeric protein comprising an intracellular binding domain and a transmembrane domain. Provided herein are CPR backbones derived from TREM2, DAP12, M6PR, MRC1, MEGF10, CLEC4L, MERTK, and FCERG. Exemplary CPR backbones of this disclosure comprise a TREM2 backbone (TREM2 ICD and TREM2 TMD), a DAP12 backbone (DAP12 ICD and DAP12 TMD), a FCERG backbone (a FCERG ICD and a CD8 TMD) and a M6PR (M6PR ICD and M6PR TMD) backbone. CPR backbones are sometimes referred to herein as “non-specific CPRs.”

In some embodiments, the CPR may further comprise a fibronectin II domain (FN2), which is about 40 amino acids in length. In particular embodiments, CPRs containing a MerTK backbone may comprise an FN2 domain.

Further provided herein are expression vectors encoding the disclosed CPRs. In some embodiments, the expression vectors encode a modified CPR having a phagocytic domain derived from TREM2. In some embodiments, the expression vector is a viral vector. In particular embodiments, the viral vector is an rAAV vector. In some embodiments, the disclosed expression vectors encode two or more CPRs.

In some aspects, methods of preparing a modified or engineering a cell comprising contacting the cell with any of the disclosed polynucleotides or vectors are provided. In some embodiments of these disclosed methods, the cell is an astrocyte, a microglia cell, or a peripheral monocyte. In some embodiments, the cell is a mammalian astrocyte (e.g., a human astrocyte). In some embodiments, the step of contacting is performed in vivo. In some embodiments, the step of contacting is performed ex vivo.

Further provided herein are uses of any of the disclosed chimeric receptors, modified cells, polynucleotides, vectors and pharmaceutical compositions for treatment of a neurodegenerative disorder, disease, or condition. Further provided are uses of any of the disclosed chimeric receptors, modified cells, polynucleotides, vectors and pharmaceutical compositions in the manufacture of a medicament for the treatment of a neurodegenerative disease in a subject in need thereof. In some embodiments, uses for the treatment of AD and PD are provided.

Further provided herein are kits that contain any of the above-described compositions, e.g., CPRs, modified cells, expression vectors, and pharmaceutical compositions. The kits may further include instructions for using the components of the kit to practice the methods of treatment and/or methods of preparing modified cells described.

### Exemplary Extracellular Binding Domains

In various embodiments, the disclosed CPRs contain an extracellular binding domain that comprises an scFv. The extracellular binding domains (EBDs) of the disclosed CPRs may comprise an amino acid sequence that is at least 85%, at least 90%, at least 95%, at least 98%, at least 99%, or at least 99.5% identical to any of the amino acid sequences that follow (SEQ ID NOs: 1-6). In some embodiments, the disclosed CPRs contain an EBD comprising an scFv having specificity against Aβ9 (anti-Aβ9 scFv). In some embodiments, the EBD comprises an scFv having specificity against Aβ 2.1.3 (anti-Aβ2.1.3 scFv). In some embodiments, the EBD comprises an scFv having specificity against tau, and is selected from anti-tau 3A6 and anti-tau PHF2. In some embodiments, the EBD comprises an scFv having specificity against α-synuclein, and is selected from anti α-synuclein 1D12. In some embodiments, the EBD comprises an scFv having specificity against TAR DNA-binding protein 43 (TDP-43), which is also associated with certain proteinopathies.

Any of the disclosed CPRs may differ relative to any of the following amino acid sequences by 1, 2, 3, 4, 5, 6, 7, 8, 9, 10, 10-15, 15-20, 20-30, 30-50, or more than 50 amino acids. These differences may comprise amino acids that have been inserted, deleted, or substituted relative to the sequence of any of SEQ ID NOs: 1-6. In some embodiments, the disclosed CPRs contain stretches of about 10, about 20, about 25, about 40, about 50, about 75, about 100, or about 125 consecutive amino acids in common with the sequence of any of SEQ ID NOs: 1-6. In some embodiments, the disclosed CPRs comprise truncations at the 5′ or 3′ end relative to any of SEQ ID NOs: 1-6.

>Anti Aβ9 scFv
(SEQ ID NO: 1)
QVTLKESGPGILQPSQTLSLTCSFSGFSLNTFGMGVSWIRQPSGK
GLEWLAHIFWDDDKHYNPSLKSRLTISKDTSNNQVFLKITTVDTA
DTATYYCVRYGFDGFPYWGQGTLVTVSAGGGGSGGGGSGGGGSDV
VMTQTPLSLPVSLGDQASISCRSNQSLVHSNGNTYLHWYLQKPGQ
SPKLLIYKVSNRFSGVPDRESGSGSGTDFTLKISRVEAEDLGFYF
CSQSTRVPWTFGGGTKLELKR
>Anti Aβ 2.1.3 (or 213) scFv
(SEQ ID NO: 2)
TRQLQQSGPELVKPGASGKISCKTSGYTFSENTMHWVKQSHGKSL
EWIGGINPNTGATYYNQKFKGKATLTVDTSSSTAYLELRSLTSAD
SAVYYCVRNGLLTLGAMDYWGQGTSVTVSSGGGGSGGGGSGGGGS
DVVMTQTPLSLPVSLGDQASISCRSSQSLVHSNGNTYLYWCLQKP
GQSPKLLIYKVSSRFSGVPDRESGSGSGTDFTLKISRVEAEDLGV
YFCSQSTHAPLTFGAGTKLELKRS
>B11 scFv (anti-Aβ)
(SEQ ID NO: 3)
DQVQLVESGGGVVQPGRSLRLSCAASGFAFSSYGMHWVRQAPGKG
LEWVAVIWFDGTKKYYTDSVKGRFTISRDNSKNTLYLQMNTLRAE
DTAVYYCARDRGIGARRGPYYMDVWGKGTTVTVSSGGGGSGGGGS
GGGGSDIQMTQSPSSLSASVGDRVTITCRASQSISSYLNWYQQKP
GKAPKLLIYAASSLQSGVPSRFSGSGSGTDFTLTISSLQPEDFAT
YYCQQSYSTPLTFGGGTKVEIKRSGGGS
>Anti tau 3A6 scFv
(SEQ ID NO: 4)
QVQLQQPGTELVKPGASVKLSCKASGYPFTNYWMHWVKQRPGQGL
EWIGNINPTNGDTNYNEKFKSKATLTVDKSSSTAYMQLSSLTSED
SAVYYCTRVTYWGQGTTLTVSSAGGGGSGGGGSGGGGSENVLTQS
PAIMSASPGEKVTMTCSASSNVNYMHWFQQESSTSPKLWIYDTSK
LASGVPGRFSGSGSGNSYSLTISSMEAEDVATYYCFQGSGYPYTF
GGGTKLEIKRL
>Anti tau PHF2 scFv
(SEQ ID NO: 5)
EVQLVQSGGGLVKPGGSLRLSCAASGFTFSSYAMSWVRQAPGKGL
EWISSITRGGSYTYFPDSVKGRFTISRDNAKNSLYLQMNSLRAED
TAVYYCARQGNYDGAWCDYWGQGTTVTVSSGGGGSGGGGSGGGGS
EIVLTQSPGTLSVSPGERATLSCRASQNIGTSIHWYQQKPGQAPR
LLIKYTSESISGIPDRESGSGSGTDFTLTISRLEPEDFAVYYCHQ
TKTWPTTFGQGTKVEIKRTLK
>Anti α-synuclein 1D12 scFv
(SEQ ID NO: 6)
QVQLQQSGAELARPGASVKLSCKASGYTFTTYWVHWVKQRPGQGL
EWIGAIYPGDGDTRYTQKFKGKATLTADESSSTAYMQLSSLASED
SAVYYCARSFAYWGQGTLVTVSAAGGGGSGGGGSGGGGSDIVMTQ
SPSSLSVSAGEKVTMSCKSSQSLLKSGNQKNYLAWYQQKPGQPPK
LLIYGASSRESGVPDRFTGSGSGTDFTLTISSVQAEDLAVYYCQN
DHSYPFTFGSGTKLEIKRLK

### Exemplary CPR Backbones

Provided herein are CPR backbones, which refer to chimeric proteins comprising an intracellular binding domain and a transmembrane domain. A complete CPR comprises the fusion of any of the disclosed scFv's to a CPR backbone. The scFv may be fused at or near the N-terminus of the CPR backbone. Alternate, the scFv may be fused at or near the C-terminus of the backbone. Provided herein are CPR backbones derived from TREM2, DAP12, M6PR, MRC1, MEGF10, CLEC4L, MERTK, and FCERG. Exemplary CPR backbones of this disclosure comprise a TREM2 backbone (TREM2 ICD and TREM2 TMD), a DAP12 backbone (DAP12 ICD and DAP12 TMD), and a M6PR backbone (M6PR ICD and M6PR TMD). In particular embodiments, a TREM2 backbone is provided. Exemplary CPR backbones of the disclosure are derived from human receptors. In some embodiments, the CPR backbones of the disclosure are derived from mouse (Mus musculus) receptors.

The backbones of the disclosed CPRs may comprise an amino acid sequence that is at least 85%, at least 90%, at least 95%, at least 98%, at least 99%, or at least 99.5% identical to any of the amino acid sequences that follow (SEQ ID NOs: 7-25). In some embodiments, the backbones of the disclosed CPRs contain stretches of about 10, about 20, about 25, about 40, or about 50 amino acids in common with the sequence of any of SEQ ID NOs: 7-25. In some embodiments, the backbones comprise truncations at the N- or C-terminus relative to any of SEQ ID NOs: 7-25. The “***” notation in the sequences below denotes the position of the scFv extracellular binding domain in a full CPR protein.

### Human-Derived Backbones:

> M6PR Backbone
(SEQ ID NO: 7)
MFPFYSCWRTGLLLLLLAVAVRESWQTTR***LKEFDYKDDDDKG
DEYDNHCGKEQRRAVVMISCNRHTLADNFNPVSEERGKVQDCFYL
FEMDSSLACSPEISHLSVGSILLVTFASLVAVYVVGGFLYQRLVV
GAKGMEQFPHLAFWQDLGNLVADGCDFVCRSKPRNVPAAYRGVGD
DQLGEESEERDDHLLPM
>DAP12 Backbone
(SEQ ID NO: 8)
MGGLEPCSRLLLLPLLLAVSGLRTR***LKEFDYKDDDDKPVQAQ
AQSDCSCSTVSPGVLAGIVMGDLVLTVLIALAVYFLGRLVPRGRG
AAEAATRKQRITETESPYQELQGQRSDVYSDLNTQRPYYK
>TREM2 Backbone
(SEQ ID NO: 9)
MEPLRLLILLFVTELSGAHTR***LKEFDYKDDDDKDHRDAGDLW
FPGESESFEDAHVEHSISRSLLEGEIPFPPTSILLLLACIFLIKI
LAASALWAAAWHGQKPGTHPPSELDCGHDPGYQLQTLPGLRDT
>CLEC4L Backbone
(SEQ ID NO: 10)
MSDSKEPRLQQLGLLEEEQLRGLGFRQTRGYKSLAGCLGHGPLVL
QLLSFTLLAGLLVQVSKVPSSISQEQSRQDAIYQNLTQLKAAVGE
LSEKSKLQEIYQELTQLKAAVGELPEKSKLQEIYQELTRLKAAVG
ELPEKSKLQEIYQELTWLKAAVGELPEKSKMQEIYQELTRLKAAV
GELPEKSKQQEIYQELTRLKAAVGELPEKSKQQEIYQELTRLKAA
VGELPEKSKQQEIYQELTQLKAAVERLCHPCPWEWTDYKDDDDKG
GGGSGGGGSGGGGSLVPSSDPGHLVEF***GGSHHHHHH
>FCERG Backbone
(SEQ ID NO: 11)
MALPVTALLLPLALLLHAARPTR***LKEFDYKDDDDKTTTPAPR
PPTPAPTIASQPLSLRPEACRPAAGGAVHTRGLDFACDIYIWAPL
AGTCGVLLLSLVITLYCRLKIQVRKAAITSYEKSDGVYTGLSTRN
QETYETLKHEKPPQ
>MRC1 Backbone
(SEQ ID NO: 12)
MRLPLLLVFASVIPGAVLLLTR***LKEFDYKDDDDKRPKIIDAK
PTHELLTTKADTRKMDPSKPSSNVAGVVIIVILLILTGAGLAAYF
FYKKRRVHLPQEGAFENTLYFNSQSSPGTSDMKDLVGNIEQNEHS
VI
>MerTK Backbone
(SEQ ID NO: 13)
MGPAPLPLLLGLFLPALWRRAITR***LKEFDYKDDDDKDIAPSV
APLNVTVFLNESSDNVDIRWMKPPTKQQDGELVGYRISHVWQSAG
ISKELLEEVGONGSRARISVQVHNATCTVRIAAVTRGGVGPFSDP
VKIFIPAHGWVDYAPSSTPAPGNADPVLIIFGCFCGFILIGLILY
ISLAIRKRVQETKFGNAFTEEDSELVVNYIAKKSFCRRAIELTLH
SLGVSEELQNKLEDVVIDRNLLILGKILGEGEFGSVMEGNLKQED
GTSLKVAVKTMKLDNSSQREIEEFLSEAACMKDFSHPNVIRLLGV
CIEMSSQGIPKPMVILPFMKYGDLHTYLLYSRLETGPKHIPLQTL
LKFMVDIALGMEYLSNRNFLHRDLAARNCMLRDDMTVCVADFGLS
KKIYSGDYYRQGRIAKMPVKWIAIESLADRVYTSKSDVWAFGVTM
WEIATRGMTPYPGVQNHEMYDYLLHGHRLKQPEDCLDELYEIMYS
CWRTDPLDRPTFSVLRLQLEKLLESLPDVRNQADVIYVNTQLLES
SEGLAQGSTLAPLDLNIDPDSIIASCTPRAAISVVTAEVHDSKPH
EGRYILNGGSEEWEDLTSAPSAAVTAEKNSVLPGERLVRNGVSWS
HSSMLPLGSSLPDELLFADDSSEGSEVLM
>MEGF10 Backbone
(SEQ ID NO: 14)
MVISLNSCLSFICLLLCHWITR***LKEFDYKDDDDKSPGWKGAR
CDQAGVIIVGNLNSLSRTSTALPADSYQIGAIAGIIILVLVVLFL
LALFIIYRHKQKGKESSMPAVTYTPAMRVVNADYTISGTLPHSNG
GNANSHYFTNPSYHTLTQCATSPHVNNRDRMTVTKSKNNQLFVNL
KNVNPGKRGPVGDCTGTLPADWKHGGYLNELGAFGLDRSYMGKSL
KDLGKNSEYNSSNCSLSSSENPYATIKDPPVLIPKSSECGYVEMK
SPARRDSPYAEINNSTSANRNVYEVEPTVSVVQGVFSNNGRLSQD
PYDLPKNSHIPCHYDLLPVRDSSSSPKQEDSGGSSSNSSSSSE
>FCERG-MRC1 Dual ICD Backbone
(SEQ ID NO: 15)
MALPVTALLLPLALLLHAARPTR***LKEFDYKDDDDKTTTPAPR
PPTPAPTIASQPLSLRPEACRPAAGGAVHTRGLDFACDIYIWAPL
AGTCGVLLLSLVITLYCRLKIQVRKAAITSYEKSDGVYTGLSTRN
QETYETLKHEKPPQVSTGGRVHLPQEGAFENTLYFNSQSSPGTSD
MKDLVGNIEQNEHSVI
>MRC1-FCERG Dual ICD Backbone
(SEQ ID NO: 16)
MRLPLLLVFASVIPGAVLLLTR***LKEFDYKDDDDKRPKIIDAK
PTHELLTTKADTRKMDPSKPSSNVAGVVIIVILLILTGAGLAAYF
FYKKRRVHLPQEGAFENTLYFNSQSSPGTSDMKDLVGNIEQNEHS
VIVSTGGRLKIQVRKAAITSYEKSDGVYTGLSTRNQETYETLKHE
KPPQ

### Mouse-Derived Backbones:

> M6PR Backbone (mouse)
(SEQ ID NO: 17)
MFPFSGCWRTELLLLLLLAVAVTR***LKEFDYKDDDDKGDEYDN
HCGKEQRRAVVMISCNRHTLAANFNPVSEERGKVQDCFYLFEMDS
SLACSPEVSHLSVGSILLVIFASLVAVYIIGGFLYQRLVVGAKGM
EQFPHLAFWQDLGNLVADGCDFVCRSKPRNVPAAYRGVGDDQLGE
ESEERDDHLLPMV
>FCERG Backbone (mouse)
(SEQ ID NO: 18)
MALPVTALLLPLALLLHAARPTR***LKEFDYKDDDDKTTTPAPR
PPTPAPTIASQPLSLRPEACRPAAGGAVHTRGLDFACDIYIWAPL
AGTCGVLLLSLVITLYCRLKIQVRKAAIASREKADAVYTGLNTRS
QETYETLKHEKPPQV
>MRC1 Backbone (mouse)
(SEQ ID NO: 19)
MRLLLLLAFISVIPVSVQLLTR***LKEFDYKDDDDKMPKIIDPV
TTHSSITTKADQRKMDPQPKGSSKAAGVVTVVLLIVIGAGVAAYF
FYKKRHALHIPQEATFENTLYFNSNLSPGTSDTKDLMGNIEQNEH
AIIV
>MerTK Backbone (mouse)
(SEQ ID NO: 20)
MVLAPLLLGLLLLPALWSGGTR***LKEFDYKDDDDKDIAPSVAP
LNITVFLNESNNILDIRWTKPPIKRQDGELVGYRISHVWESAGTY
KELSEEVSQNGSWAQIPVQIHNATCTVRIAAITKGGIGPFSEPVN
IIIPEDIHSKVDYAPSSTPAPGNTDSMFIILGCFCGFILIGLILC
ISLALRRRVQETKFGGAFSEEDSQLVVNYRAKKSFCRRAIELTLQ
SLGVSEELQNKLEDVVIDRNLLVLGKVLGEGEFGSVMEGNLKQED
GTSQKVAVKTMKLDNESQREIEEFLSEAACMKDFNHPNVIRLLGV
CIELSSQGIPKPMVILPFMKYGDLHTFLLYSRLNTGPKYIHLQTL
LKFMMDIAQGMEYLSNRNFLHRDLAARNCMLRDDMTVCVADFGLS
KKIYSGDYYRQGRIAKMPVKWIAIESLADRVYTSKSDVWAFGVTM
WEITTRGMTPYPGVQNHEMYDYLLHGHRLKQPEDCLDELYDIMYS
CWSADPLDRPTFSVLRLQLEKLSESLPDAQDKESIIYINTQLLES
CEGIANGPSLTGLDMNIDPDSIIASCTPGAAVSVVTAEVHENNLR
EERYILNGGNEEWEDVSSTPFAAVTPEKDGVLPEDRLTKNGVSWS
HHSTLPLGSPSPDELLFVDDSLEDSEVLMV
>MEGF10 Backbone (mouse)
(SEQ ID NO: 21)
MAISSSSCLGLICSLLCHWVTR***LKEFDYKDDDDKSPGWKGAR
CDQAGVIIVGNLNSLSRTSTALPADSYQIGAIAGIVVLVLVVLFL
LALFIIYRHKQKRKESSMPAVTYTPAMRVINADYTIAETLPHSNG
GNANSHYFTNPSYHTLSQCATSPHVNNRDRMTIAKSKNNQLFVNL
KNVNPGKRGTLVDCTGTLPADWKQGGYLNELGAFGLDRSYMGKSL
KDLGKNSEYNSSTCSLSSSENPYATIKDPPALLPKSSECGYVEMK
SPARRDSPYAEINNSTPANRNVYEVEPTVSVVQGVFSNSGHVTQD
PYDLPKNSHIPCHYDLLPVRDSSSSPKREDGGGSNSTSSNSTSSS
SSSSEV
>DAP12 Backbone (mouse)
(SEQ ID NO: 22)
MGALEPSWCLLFLPVLLTVGGLSTR***LKEFDYKDDDDKPVQAQ
SDTFPRCDCSSVSPGVLAGIVLGDLVLTLLIALAVYSLGRLVSRG
QGTAEGTRKQHIAETESPYQELQGQRPEVYSDLNTQRQYYRV
>TREM2 Backbone (mouse)
(SEQ ID NO: 23)
MGPLHQFLLLLITALSQALTR***LKEFDYKDDDDKDDQDAGDLW
VPEESSSFEGAQVEHSTSRNQETSFPPTSILLLLACVLLSKFLAA
SILWAVARGRQKPGTPVVRGLDCGQDAGHQLQILTGPGGT
>FCERG-MRC1 Dual ICD Backbone (mouse)
(SEQ ID NO: 24)
MALPVTALLLPLALLLHAARPTR***LKEFDYKDDDDKTTTPAPR
PPTPAPTIASQPLSLRPEACRPAAGGAVHTRGLDFACDIYIWAPL
AGTCGVLLLSLVITLYCRLKIQVRKAAIASREKADAVYTGLNTRS
QETYETLKHEKPPQVSTGGHALHIPQEATFENTLYFNSNLSPGTS
DTKDLMGNIEQNEHAIIV
>MRC1-FCERG Dual ICD Backbone (mouse)
(SEQ ID NO: 25)
MALPVTALLLPLALLLHAARPTR***LKEFDYKDDDDKMPKIIDP
VTTHSSITTKADQRKMDPQPKGSSKAAGVVTVVLLIVIGAGVAAY
FFYKKRHALHIPQEATFENTLYFNSNLSPGTSDTKDLMGNIEQNE
HAIIVSTGGRLKIQVRKAAIASREKADAVYTGLNTRSQETYETLK
HEKPPQV

### Exemplary Chimeric Phagocytic Receptors

Provided herein are chimeric phagocytic receptors that comprise any of the disclosed CPR backbones and an extracellular binding domain containing any of the disclosed scFv's. The component parts of a CPR as disclosed herein can be selected and arranged in various combinations to provide a desired engulfment phenotype to a cell. In various embodiments, the disclosed chimeric receptors comprise an amino acid sequence that is at least 85%, at least 90%, at least 95%, at least 98%, at least 99%, or at least 99.5% identical to any of the amino acid sequences that follow (SEQ ID NOs: 26-43). Any of the disclosed chimeric receptors may differ relative to any of the following amino acid sequences by 1, 2, 3, 4, 5, 6, 7, 8, 9, 10, 10-15, 15-20, 20-30, 30-50, or more than 50 amino acids. These differences may comprise amino acids that have been inserted, deleted, or substituted relative to the sequence of any of SEQ ID NOs: 26-43. In some embodiments, the disclosed chimeric receptors contain stretches of about 10, about 20, about 25, about 40, about 50, about 75, about 100, about 125, about 150, about 175, or about 180 amino acids in common with the sequence of any of SEQ ID NOs: 26-43. In some embodiments, the disclosed chimeric receptors comprise truncations at the N- or C-terminus relative to any of SEQ ID NOs: 26-43. For instance, this disclosure provides CPRs that are truncated by 1, 2, 3, 4, 5 or more than 5 amino acids at the N- or C-terminus of any one of SEQ ID NOs: 26-43.

The CPRs of the disclosure may comprise any of the following architectures or structure: NH₂-[scFv EBD]-x-[hinge region]-[TMD]-[ICD]-COOH; NH₂-[scFv EBD]-x-[hinge region]-[TMD]-x-[ICD]-COOH; NH₂-[scFv EBD]-x-[fibronectin II domain]-x-[hinge region]-[TMD]-[ICD]-COOH; NH₂-[scFv EBD]-x-[hinge region]-[TMD]-[ICD 1]-[ICD 2]-COOH; NH₂-[scFv EBD]-x-[hinge region]-[TMD]-x-[ICD 1]-x-[ICD 2]-COOH; and NH₂-[ICD]-[TMD]-[hinge region]-x-[scFv EBD]-COOH, wherein each instance of “]-[” represents an optional linker and each instance of “x” represents an optional restriction enzyme site. In particular embodiments, the CPRs of the disclosure comprise the structure NH₂-[scFv EBD]-x-[hinge region]-[TMD]-[ICD]-COOH.

In some embodiments, the CPRs comprise a chimeric receptor selected from any of the following (scFv Backbone): Aβ9_TREM2, Aβ9_FCERG, Aβ9_MRC1, Aβ9_MerTK, Aβ9_MEGF10, CLEC4L_Aβ9, Aβ9_FCERG-MRC1, Aβ9_MRC1-FCERG; 2.1.3_TREM2, 2.1.3_FCERG, 2.1.3_MRC1, 2.1.3_MerTK, 2.1.3_MEGF10, CLEC4L_2.1.3, 2.1.3_FCERG-MRC1, 2.1.3_MRC1-FCERG; 3A6_TREM2, 3A6_FCERG, 3A6_MRC1, 3A6_MerTK, 3A6_MEGF10, CLEC4L_3A6, 3A6_FCERG-MRC1, 3A6_MRC1-FCERG; PHF2_TREM2, PHF2_FCERG, PHF2_MRC1, PHF2_MerTK, PHF2_MEGF10, CLEC4L_PHF2, PHF2_FCERG-MRC1, PHF2_MRC1-FCERG; 1D12_TREM2, 1D12_FCERG, 1D12_MRC1, 1D12_MerTK, 1D12_MEGF10, CLEC4L_1D12, 1D12_FCERG-MRC1, 1D12_MRC1-FCERG; B11_TREM2, B11_FCERG, B11_MRC1, B11_MerTK, B11_MEGF10, CLEC4L_B11, B11_FCERG-MRC1, and B11_MRC1-FCERG.

> Aβ9_TREM2
(SEQ ID NO: 26)
MEPLRLLILLFVTELSGAHTRQVTLKESGPGILQPSQTLSLTCSF
SGFSLNTFGMGVSWIRQPSGKGLEWLAHIFWDDDKHYNPSLKSRL
TISKDTSNNQVFLKITTVDTADTATYYCVRYGFDGFPYWGQGTLV
TVSAGGGGSGGGGSGGGGSDVVMTQTPLSLPVSLGDQASISCRSN
QSLVHSNGNTYLHWYLQKPGQSPKLLIYKVSNRFSGVPDRFSGSG
SGTDFTLKISRVEAEDLGFYFCSQSTRVPWTFGGGTKLELKRLKE
FDYKDDDDKDHRDAGDLWFPGESESFEDAHVEHSISRSLLEGEIP
FPPTSILLLLACIFLIKILAASALWAAAWHGQKPGTHPPSELDCG
HDPGYQLQTLPGLRDT
>CLEC4L_Aβ9
(SEQ ID NO: 27)
MSDSKEPRLQQLGLLEEEQLRGLGFRQTRGYKSLAGCLGHGPLVL
QLLSFTLLAGLLVQVSKVPSSISQEQSRQDAIYQNLTQLKAAVGE
LSEKSKLQEIYQELTQLKAAVGELPEKSKLQEIYQELTRLKAAVG
ELPEKSKLQEIYQELTWLKAAVGELPEKSKMQEIYQELTRLKAAV
GELPEKSKQQEIYQELTRLKAAVGELPEKSKQQEIYQELTRLKAA
VGELPEKSKQQEIYQELTQLKAAVERLCHPCPWEWTDYKDDDDKG
GGGSGGGGSGGGGSLVPSSDPGHLVEFQVTLKESGPGILQPSQTL
SLTCSFSGFSLNTFGMGVSWIRQPSGKGLEWLAHIFWDDDKHYNP
SLKSRLTISKDTSNNQVFLKITTVDTADTATYYCVRYGFDGFPYW
GQGTLVTVSAGGGGSGGGGSGGGGSDVVMTQTPLSLPVSLGDQAS
ISCRSNQSLVHSNGNTYLHWYLQKPGQSPKLLIYKVSNRFSGVPD
RFSGSGSGTDFTLKISRVEAEDLGFYFCSQSTRVPWTFGGGTKLE
LKRGGSHHHHHH
>Aβ9_FCERG
(SEQ ID NO: 28)
MALPVTALLLPLALLLHAARPTRQVTLKESGPGILQPSQTLSLTC
SFSGFSLNTFGMGVSWIRQPSGKGLEWLAHIFWDDDKHYNPSLKS
RLTISKDTSNNQVFLKITTVDTADTATYYCVRYGFDGFPYWGQGT
LVTVSAGGGGSGGGGSGGGGSDVVMTQTPLSLPVSLGDQASISCR
SNQSLVHSNGNTYLHWYLQKPGQSPKLLIYKVSNRFSGVPDRESG
SGSGTDFTLKISRVEAEDLGFYFCSQSTRVPWTFGGGTKLELKRL
KEFDYKDDDDKTTTPAPRPPTPAPTIASQPLSLRPEACRPAAGGA
VHTRGLDFACDIYIWAPLAGTCGVLLLSLVITLYCRLKIQVRKAA
ITSYEKSDGVYTGLSTRNQETYETLKHEKPPQ
>Aβ9_MRC1
(SEQ ID NO: 29)
MRLPLLLVFASVIPGAVLLLTRQVTLKESGPGILQPSQTLSLTCS
FSGFSLNTFGMGVSWIRQPSGKGLEWLAHIFWDDDKHYNPSLKSR
LTISKDTSNNQVFLKITTVDTADTATYYCVRYGFDGFPYWGQGTL
VTVSAGGGGSGGGGSGGGGSDVVMTQTPLSLPVSLGDQASISCRS
NQSLVHSNGNTYLHWYLQKPGQSPKLLIYKVSNRFSGVPDRESGS
GSGTDFTLKISRVEAEDLGFYFCSQSTRVPWTFGGGTKLELKRL
KEFDYKDDDDKRPKIIDAKPTHELLTTKADTRKMDPSKPSSNVAG
VVIIVILLILTGAGLAAYFFYKKRRVHLPQEGAFENTLYFNSQSS
PGTSDMKDLVGNIEQNEHSVI
> Aβ9_MerTK
(SEQ ID NO: 30)
MGPAPLPLLLGLFLPALWRRAITRQVTLKESGPGILQPSQTLSLT
CSFSGFSLNTFGMGVSWIRQPSGKGLEWLAHIFWDDDKHYNPSLK
SRLTISKDTSNNQVFLKITTVDTADTATYYCVRYGFDGFPYWGQG
TLVTVSAGGGGSGGGGSGGGGSDVVMTQTPLSLPVSLGDQASISC
RSNQSLVHSNGNTYLHWYLQKPGQSPKLLIYKVSNRFSGVPDRES
GSGSGTDFTLKISRVEAEDLGFYFCSQSTRVPWTFGGGTKLELKR
LKEFDYKDDDDKDIAPSVAPLNVTVFLNESSDNVDIRWMKPPTKQ
QDGELVGYRISHVWQSAGISKELLEEVGQNGSRARISVQVHNATC
TVRIAAVTRGGVGPFSDPVKIFIPAHGWVDYAPSSTPAPGNADPV
LIIFGCFCGFILIGLILYISLAIRKRVQETKFGNAFTEEDSELVV
NYIAKKSFCRRAIELTLHSLGVSEELQNKLEDVVIDRNLLILGKI
LGEGEFGSVMEGNLKQEDGTSLKVAVKTMKLDNSSQREIEEFLSE
AACMKDFSHPNVIRLLGVCIEMSSQGIPKPMVILPFMKYGDLHTY
LLYSRLETGPKHIPLQTLLKFMVDIALGMEYLSNRNFLHRDLAAR
NCMLRDDMTVCVADFGLSKKIYSGDYYRQGRIAKMPVKWIAIESL
ADRVYTSKSDVWAFGVTMWEIATRGMTPYPGVQNHEMYDYLLHGH
RLKQPEDCLDELYEIMYSCWRTDPLDRPTFSVLRLQLEKLLESLP
DVRNQADVIYVNTQLLESSEGLAQGSTLAPLDLNIDPDSIIASCT
PRAAISVVTAEVHDSKPHEGRYILNGGSEEWEDLTSAPSAAVTAE
KNSVLPGERLVRNGVSWSHSSMLPLGSSLPDELLFADDSSEGSEV
LM
> Aβ9_MEGF10
(SEQ ID NO: 31)
MVISLNSCLSFICLLLCHWITRQVTLKESGPGILQPSQTLSLTCS
FSGFSLNTFGMGVSWIRQPSGKGLEWLAHIFWDDDKHYNPSLKSR
LTISKDTSNNQVFLKITTVDTADTATYYCVRYGFDGFPYWGQGTL
VTVSAGGGGSGGGGSGGGGSDVVMTQTPLSLPVSLGDQASISCRS
NQSLVHSNGNTYLHWYLQKPGQSPKLLIYKVSNRFSGVPDRESGS
GSGTDFTLKISRVEAEDLGFYFCSQSTRVPWTFGGGTKLELKRL
KEFDYKDDDDKSPGWKGARCDQAGVIIVGNLNSLSRTSTALPADS
YQIGAIAGIIILVLVVLFLLALFIIYRHKQKGKESSMPAVTYTPA
MRVVNADYTISGTLPHSNGGNANSHYFTNPSYHTLTQCATSPHVN
NRDRMTVTKSKNNQLFVNLKNVNPGKRGPVGDCTGTLPADWKHGG
YLNELGAFGLDRSYMGKSLKDLGKNSEYNSSNCSLSSSENPYATI
KDPPVLIPKSSECGYVEMKSPARRDSPYAEINNSTSANRNVYEVE
PTVSVVQGVFSNNGRLSQDPYDLPKNSHIPCHYDLLPVRDSSSSP
KQEDSGGSSSNSSSSSE
> Aβ9_FCERG-MRC1
(SEQ ID NO: 32)
MALPVTALLLPLALLLHAARPTRQVTLKESGPGILQPSQTLSLTC
SFSGFSLNTFGMGVSWIRQPSGKGLEWLAHIFWDDDKHYNPSLKS
RLTISKDTSNNQVFLKITTVDTADTATYYCVRYGFDGFPYWGQGT
LVTVSAGGGGSGGGGSGGGGSDVVMTQTPLSLPVSLGDQASISCR
SNQSLVHSNGNTYLHWYLQKPGQSPKLLIYKVSNRFSGVPDRESG
SGSGTDFTLKISRVEAEDLGFYFCSQSTRVPWTFGGGTKLELKRL
KEFDYKDDDDKTTTPAPRPPTPAPTIASQPLSLRPEACRPAAGGA
VHTRGLDFACDIYIWAPLAGTCGVLLLSLVITLYCRLKIQVRKAA
ITSYEKSDGVYTGLSTRNQETYETLKHEKPPQVSTGGRVHLPQEG
AFENTLYFNSQSSPGTSDMKDLVGNIEQNEHSVI
> Aβ9_MRC1-FCERG
(SEQ ID NO: 33)
MRLPLLLVFASVIPGAVLLLTRQVTLKESGPGILQPSQTLSLTCS
FSGFSLNTFGMGVSWIRQPSGKGLEWLAHIFWDDDKHYNPSLKSR
LTISKDTSNNQVFLKITTVDTADTATYYCVRYGFDGFPYWGQGTL
VTVSAGGGGSGGGGSGGGGSDVVMTQTPLSLPVSLGDQASISCRS
NQSLVHSNGNTYLHWYLQKPGQSPKLLIYKVSNRFSGVPDRFSGS
GSGTDFTLKISRVEAEDLGFYFCSQSTRVPWTFGGGTKLELKRLK
EFDYKDDDDKRPKIIDAKPTHELLTTKADTRKMDPSKPSSNVAGV
VIIVILLILTGAGLAAYFFYKKRRVHLPQEGAFENTLYFNSQSSP
GTSDMKDLVGNIEQNEHSVIVSTGGRLKIQVRKAAITSYEKSDGV
YTGLSTRNQETYETLKHEKPPQ
>2.1.3_TREM2
(SEQ ID NO: 34)
MEPLRLLILLFVTELSGAHTRTRQLQQSGPELVKPGASGKISCKT
SGYTFSENTMHWVKQSHGKSLEWIGGINPNTGATYYNQKFKGKAT
LTVDTSSSTAYLELRSLTSADSAVYYCVRNGLLTLGAMDYWGQGT
SVTVSSGGGGSGGGGSGGGGSDVVMTQTPLSLPVSLGDQASISCR
SSQSLVHSNGNTYLYWCLQKPGQSPKLLIYKVSSRFSGVPDRESG
SGSGTDFTLKISRVEAEDLGVYFCSQSTHAPLTFGAGTKLELKRS
LKEFDYKDDDDKDHRDAGDLWFPGESESFEDAHVEHSISRSLLEG
EIPFPPTSILLLLACIFLIKILAASALWAAAWHGQKPGTHPPSEL
DCGHDPGYQLQTLPGLRDT
>2.1.3_FCERG
(SEQ ID NO: 35)
MALPVTALLLPLALLLHAARPTRTRQLQQSGPELVKPGASGKISC
KTSGYTFSENTMHWVKQSHGKSLEWIGGINPNTGATYYNQKFKGK
ATLTVDTSSSTAYLELRSLTSADSAVYYCVRNGLLTLGAMDYWGQ
GTSVTVSSGGGGSGGGGSGGGGSDVVMTQTPLSLPVSLGDQASIS
CRSSQSLVHSNGNTYLYWCLQKPGQSPKLLIYKVSSRFSGVPDRE
SGSGSGTDFTLKISRVEAEDLGVYFCSQSTHAPLTFGAGTKLEL
KRSLKEFDYKDDDDKTTTPAPRPPTPAPTIASQPLSLRPEACRPA
AGGAVHTRGLDFACDIYIWAPLAGTCGVLLLSLVITLYCRLKIQV
RKAAITSYEKSDGVYTGLSTRNQETYETLKHEKPPQ
>3A6_TREM2
(SEQ ID NO: 36)
MEPLRLLILLFVTELSGAHTRQVQLQQPGTELVKPGASVKLSCKA
SGYPFTNYWMHWVKQRPGQGLEWIGNINPTNGDTNYNEKFKSKAT
LTVDKSSSTAYMQLSSLTSEDSAVYYCTRVTYWGQGTTLTVSSAG
GGGSGGGGSGGGGSENVLTQSPAIMSASPGEKVTMTCSASSNVNY
MHWFQQESSTSPKLWIYDTSKLASGVPGRESGSGSGNSYSLTISS
MEAEDVATYYCFQGSGYPYTFGGGTKLEIKRLLKEFDYKDDDDKD
HRDAGDLWFPGESESFEDAHVEHSISRSLLEGEIPFPPTSILLLL
ACIFLIKILAASALWAAAWHGQKPGTHPPSELDCGHDPGYQLQTL
PGLRDT
>3A6_FCERG
(SEQ ID NO: 37)
MALPVTALLLPLALLLHAARPTRQVQLQQPGTELVKPGASVKLSC
KASGYPFTNYWMHWVKQRPGQGLEWIGNINPTNGDTNYNEKFKSK
ATLTVDKSSSTAYMQLSSLTSEDSAVYYCTRVTYWGQGTTLTVSS
AGGGGSGGGGSGGGGSENVLTQSPAIMSASPGEKVTMTCSASSNV
NYMHWFQQESSTSPKLWIYDTSKLASGVPGRFSGSGSGNSYSLTI
SSMEAEDVATYYCFQGSGYPYTFGGGTKLEIKRLLKEFDYKDDDD
KTTTPAPRPPTPAPTIASQPLSLRPEACRPAAGGAVHTRGLDFAC
DIYIWAPLAGTCGVLLLSLVITLYCRLKIQVRKAAITSYEKSDGV
YTGLSTRNQETYETLKHEKPPQ
> PHF2_TREM2
(SEQ ID NO: 38)
MEPLRLLILLFVTELSGAHTREVQLVQSGGGLVKPGGSLRLSCAA
SGFTFSSYAMSWVRQAPGKGLEWISSITRGGSYTYFPDSVKGRFT
ISRDNAKNSLYLQMNSLRAEDTAVYYCARQGNYDGAWCDYWGQGT
TVTVSSGGGGSGGGGSGGGGSEIVLTQSPGTLSVSPGERATLSCR
ASQNIGTSIHWYQQKPGQAPRLLIKYTSESISGIPDRFSGSGSGT
DFTLTISRLEPEDFAVYYCHQTKTWPTTFGQGTKVEIKRTLKLKE
FDYKDDDDKDHRDAGDLWFPGESESFEDAHVEHSISRSLLEGEIP
FPPTSILLLLACIFLIKILAASALWAAAWHGQKPGTHPPSELDCG
HDPGYQLQTLPGLRDT
> PHF2_FCERG
(SEQ ID NO: 39)
MALPVTALLLPLALLLHAARPTREVQLVQSGGGLVKPGGSLRLSC
AASGFTFSSYAMSWVRQAPGKGLEWISSITRGGSYTYFPDSVKGR
FTISRDNAKNSLYLQMNSLRAEDTAVYYCARQGNYDGAWCDYWGQ
GTTVTVSSGGGGSGGGGSGGGGSEIVLTQSPGTLSVSPGERATLS
CRASQNIGTSIHWYQQKPGQAPRLLIKYTSESISGIPDRESGSGS
GTDFTLTISRLEPEDFAVYYCHQTKTWPTTFGQGTKVEIKRTLKL
KEFDYKDDDDKTTTPAPRPPTPAPTIASQPLSLRPEACRPAAGGA
VHTRGLDFACDIYIWAPLAGTCGVLLLSLVITLYCRLKIQVRKAA
ITSYEKSDGVYTGLSTRNQETYETLKHEKPPQ
> 1D12_TREM2
(SEQ ID NO: 40)
MEPLRLLILLFVTELSGAHTRQVQLQQSGAELARPGASVKLSCKA
SGYTFTTYWVHWVKQRPGQGLEWIGAIYPGDGDTRYTQKFKGKAT
LTADESSSTAYMQLSSLASEDSAVYYCARSFAYWGQGTLVTVSAA
GGGGSGGGGSGGGGSDIVMTQSPSSLSVSAGEKVTMSCKSSQSLL
KSGNQKNYLAWYQQKPGQPPKLLIYGASSRESGVPDRFTGSGSGT
DFTLTISSVQAEDLAVYYCONDHSYPFTFGSGTKLEIKRLKLKEF
DYKDDDDKDHRDAGDLWFPGESESFEDAHVEHSISRSLLEGEIPF
PPTSILLLLACIFLIKILAASALWAAAWHGQKPGTHPPSELDCGH
DPGYQLQTLPGLRDT
>1D12_FCERG
(SEQ ID NO: 41)
MALPVTALLLPLALLLHAARPTRQVQLQQSGAELARPGASVKLSC
KASGYTFTTYWVHWVKQRPGQGLEWIGAIYPGDGDTRYTQKFKGK
ATLTADESSSTAYMQLSSLASEDSAVYYCARSFAYWGQGTLVTVS
AAGGGGSGGGGSGGGGSDIVMTQSPSSLSVSAGEKVTMSCKSSQS
LLKSGNQKNYLAWYQQKPGQPPKLLIYGASSRESGVPDRFTGSGS
GTDFTLTISSVQAEDLAVYYCONDHSYPFTFGSGTKLEIKRLKLK
EFDYKDDDDKTTTPAPRPPTPAPTIASQPLSLRPEACRPAAGGAV
HTRGLDFACDIYIWAPLAGTCGVLLLSLVITLYCRLKIQVRKAAI
TSYEKSDGVYTGLSTRNQETYETLKHEKPPQ
>B11_TREM2 Backbone
(SEQ ID NO: 42)
MEPLRLLILLFVTELSGAHTRDQVQLVESGGGVVQPGRSLRLSCA
ASGFAFSSYGMHWVRQAPGKGLEWVAVIWFDGTKKYYTDSVKGRF
TISRDNSKNTLYLQMNTLRAEDTAVYYCARDRGIGARRGPYYMDV
WGKGTTVTVSSGGGGSGGGGSGGGGSDIQMTQSPSSLSASVGDRV
TITCRASQSISSYLNWYQQKPGKAPKLLIYAASSLQSGVPSRFSG
SGSGTDFTLTISSLOPEDFATYYCQQSYSTPLTFGGGTKVEIKRS
GGGSLKEFDYKDDDDKDHRDAGDLWFPGESESFEDAHVEHSISRS
LLEGEIPFPPTSILLLLACIFLIKILAASALWAAAWHGQKPGTHP
PSELDCGHDPGYQLQTLPGLRDT
>B11_FCERG
(SEQ ID NO: 43)
MALPVTALLLPLALLLHAARPTRDQVQLVESGGGVVQPGRSLRLS
CAASGFAFSSYGMHWVRQAPGKGLEWVAVIWFDGTKKYYTDSVKG
RFTISRDNSKNTLYLQMNTLRAEDTAVYYCARDRGIGARRGPYYM
DVWGKGTTVTVSSGGGGSGGGGSGGGGSDIQMTQSPSSLSASVGD
RVTITCRASQSISSYLNWYQQKPGKAPKLLIYAASSLQSGVPSRF
SGSGSGTDFTLTISSLQPEDFATYYCQQSYSTPLTFGGGTKVEIK
RSGGGSLKEFDYKDDDDKTTTPAPRPPTPAPTIASQPLSLRPEAC
RPAAGGAVHTRGLDFACDIYIWAPLAGTCGVLLLSLVITLYCRLK
IQVRKAAITSYEKSDGVYTGLSTRNQETYETLKHEKPPQ

### Polynucleotides and Vectors

Provided herein are polynucleotides encoding any of the disclosed CPRs, and expression vectors comprising any of these polynucleotides. In some embodiments, the polynucleotides comprise cDNA molecules.

Further provided herein are expression vectors comprising any of the disclosed polynucleotides. As used herein, the term “vector” refers to a nucleic acid segment (e.g., a plasmid or recombinant viral genome). In some embodiments, vectors that allow long-term integration of a transgene and propagation to daughter cells are utilized. Examples include viral vectors such as adenovirus, AAV, and lentiviral vectors. In some embodiments, vectors that allow transient integration of a transgene are utilized. In particular embodiments, the expression vector is an rAAV vector. The rAAV vectors may contain a transgene encoding the CPR flanked by one or more inverted terminal repeats (ITRs). In some embodiments, the expression vector is a lentiviral vector. In some embodiments, the disclosed expression vectors encode two or more CPRs.

The polynucleotide or vector encoding the CPR may comprise a sequence encoding a secretion signal peptide, which are cis-acting sequences that direct trafficking of the CPR to the cell membrane. Polypeptides that are targeted to a membrane bound compartment such as a secretory vesicle may be retained in the plasma membrane, as is the case for the disclosed CPRs. The signal peptide is optionally cleaved from the N-terminus of the extracellular binding domain during cellular processing and trafficking of the CPR to the cell membrane. The secretion signal peptide (“SP”) may be derived from the same protein as the intracellular phagocytic signaling domain (or “ICD”). Accordingly, a polynucleotide encoding a CPR expressing an MRC1 ICD may comprise an MRC1 signal peptide. Likewise, a polynucleotide encoding a CPR expressing a TREM2 ICD may comprise a TREM2 signal peptide. In exemplary embodiments, the polynucleotide comprises a TREM2 signal peptide. In other embodiments, the signal peptide is selected from a MEGF10 signal peptide, a DAP12 signal peptide, a MERTK (or MerTK) signal peptide, a CD8 signal peptide, and a M6PR signal peptide. In some embodiments, the signal peptide comprises a CD209 peptide which is also known as “DC-SIGN” (Dendritic Cell-Specific Intercellular adhesion molecule-3-Grabbing Non-integrin) or CLEC4L.

The vectors may include, or exclude, the sequence of affinity tags for the purpose of detection and purification of the fusion proteins of the disclosure. Examples of affinity tags include FLAG tag, polyhistidine-tag (His₆ tag), myc-tag, Strep-tag, E-tag, hemagglutinin tag, T7, S-tag, HSV, VSV-G, anti-Xpress, and VS-tag.

Accordingly, in some embodiments, the disclosed polynucleotides and expression vectors proteins may comprise i) a transgene encoding the CPR, ii) a secretion signal peptide sequence, and iii) one or more affinity tags, such as a FLAG tag or a polyhistidine (His₆) tag. In particular embodiments, the disclosed vectors comprise one or two FLAG tags. In particular embodiments, the vectors comprise a FLAG tag at the C-terminus of the vector. In particular embodiments, the vectors comprise a FLAG tag positioned C-terminal of the CPR. In some embodiments, the vectors comprise one or two FLAG tags. In some embodiments, the FLAG tag sequence is DYKDDDDK (SEQ ID NO: 44).

To achieve appropriate or enhanced expression levels of the CPR, any of a number of heterologous promoters suitable for use in the selected host cell may be employed. The promoter may be, for example, a constitutive promoter, tissue-specific promoter, inducible promoter, or a synthetic promoter. Inducible promoters and/or regulatory elements may also be contemplated for achieving appropriate expression levels of the heterologous peptide. Non-limiting examples of suitable inducible promoters include the CBA promoter and those promoters from genes such as cytochrome P450 genes, heat shock protein genes, metallothionein genes, and hormone-inducible genes, such as the estrogen gene promoter.

Tissue-specific promoters and/or regulatory elements are also contemplated herein. In certain embodiments, the heterologous promoters of the disclosed chimeric receptors are active in brain cells, such as human brain cells (e.g. human neurons and glia). In various embodiments, the heterologous promoter is active in astrocyte cells. In some embodiments, the heterologous promoter is active in microglial cells. The disclosed vectors may comprise a heterologous promoter selected from a CD68 promoter, a Glial Fibrillary Acidic protein (GFAP) promoter, a CD8 promoter, a CMV enhancer/chicken Beta-actin (CAG) promoter, a Microtubule-associated protein 2 (MAP2) promoter or a synapsin 1 (SYN) promoter. In particular embodiments, the promoter is a CD68 promoter. In some embodiments, the promoter is a GFAP promoter. In some embodiments, the promoter is a CD8 promoter.

Additional exemplary promoters active in human brain cells include, but are not limited to, the human synapsin 1 gene promoter (SYN), the hybrid CMV enhancer/chicken β-actin (CAG) promoter, Glial Fibrillary Acidic protein (GFAP) promoter, Microtubule-associated protein 2 (MAP2) promoter, and the platelet-derived growth factor-β chain promoter (1500 bp). These promoters are described in, e.g., Kugler et al. Virology, 311(1):89-95 (2003) and Morelli et al. J Gen Virol., 80(Pt 3):571-83 (1999), which are incorporated herein by reference. Non-limiting examples of such promoters that may be used include species-specific promoters, such as human-specific promoters.

Synthetic promoters are also contemplated herein. A synthetic promoter may comprise, for example, regions of known promoters, regulatory elements, transcription factor binding sites, enhancer elements, repressor elements, and the like.

In some embodiments, the disclosed polynucleotides and expression vectors proteins may comprise one or more cleavage sites, such as furin cleavage sites.

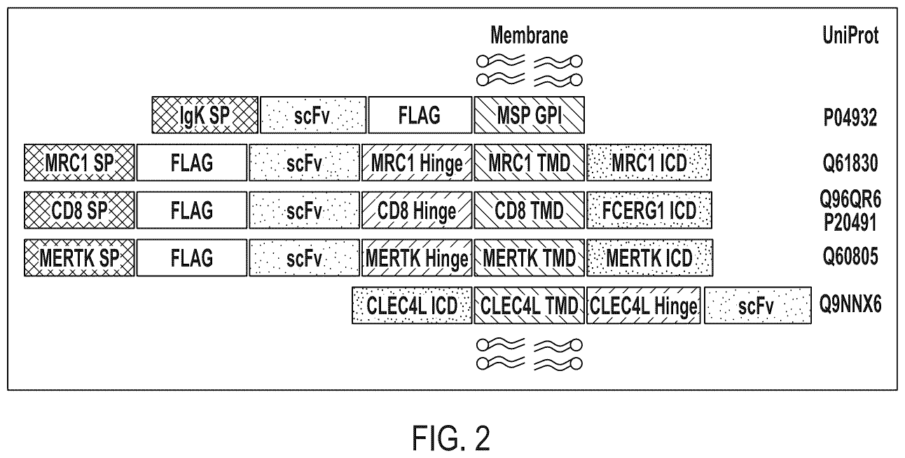

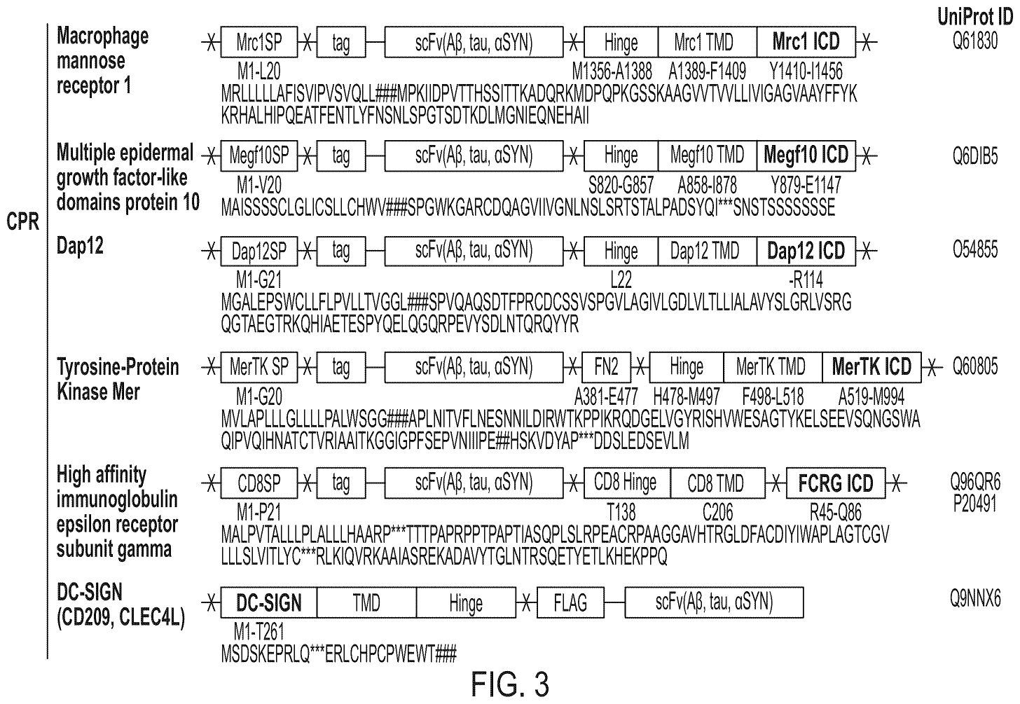

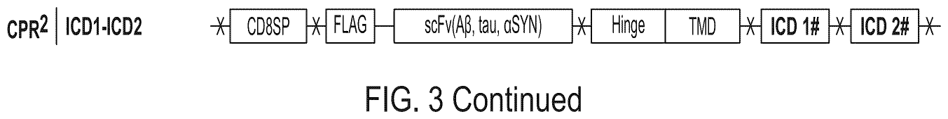

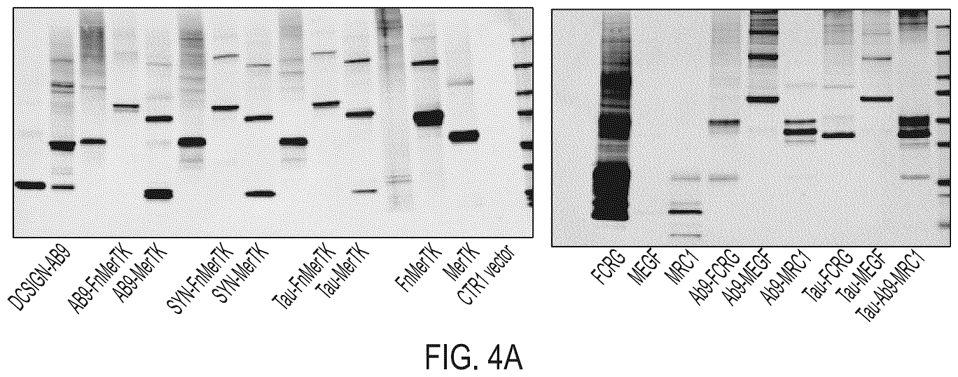

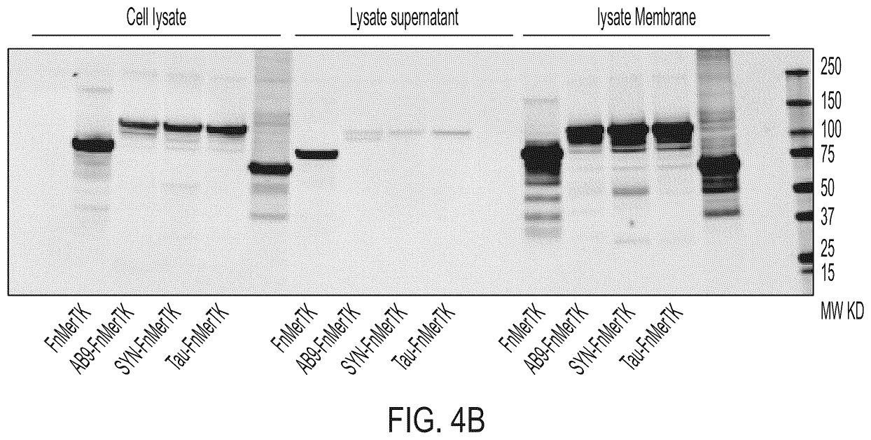

The expression vectors of the disclosure encoding the backbones of the disclosed CPRs may comprise any of the following peptide-encoding structures: 5′-[SP]-x-[tag]-[scFv EBD]-x-[hinge region]-[TMD]-[ICD]-3′; 5′-[SP]-x-[tag]-[scFv EBD]-x-[hinge region]-[TMD]-x-[ICD]-3′; 5′-[SP]-x-[tag]-[scFv EBD]-x-[fibronectin II domain]-x-[hinge region]-[TMD]-[ICD]-3′; 5′-[SP]-x-[tag]-[scFv EBD]-x-[hinge region]-[TMD]-[ICD 1]-[ICD 2]-3′; 5′-[SP]-x-[tag]-[scFv EBD]-x-[hinge region]-[TMD]-x-[ICD 1]-x-[ICD 2]-3′; and 5′-[ICD]-[TMD]-[hinge region]-x-[scFv EBD]-3′, wherein each instance of “]-[” represents an optional linker-encoding sequence and each instance of “x” represents an optional restriction enzyme site. In particular embodiments, the cleavage sites are furin cleavage sites. “[SP]” represents a signal peptide-encoding sequence, and [tag] represents a tag, such as a FLAG tag. In particular embodiments, the expression vector comprises the structure 5′-[SP]-x-[tag]-[scFv EBD]-x-[hinge region]-[TMD]-[ICD]-3′. FIGS. 2 and 3 provide illustrative CPR-encoding vectors of the disclosure. FIG. 3 provides contiguous segments of exemplary amino acid sequences for each of the exemplary CPR peptides, shown as SEQ ID NOs: 57-62, from top to bottom.

Many of the sequences that follow (SEQ ID NOs: 45-54) are illustrated with poly(A) tails and FLAG tags or other tags. However, as can readily be visualized by one skilled in the art, in some embodiments, the disclosed exemplary vectors do not contain any one of these tags. In some embodiments, any of the CPR backbone vectors set forth in SEQ ID NOs: 45-54 comprise a tag other than a FLAG tag. In some embodiments, the FLAG tag of any of the vectors of SEQ ID NOs: 45-54 is substituted with an alternative tag, such as a myc-tag, Strep-tag, E-tag, hemagglutinin tag, T7 tag, S-tag, HSV, VSV-G, anti-Xpress, and VS-tag.

In some embodiments, the polynucleotide and/or vector of the disclosure comprises a nucleic acid sequence having at least 85%, at least 90%, at least 92.5%, at least 95%, at least 98%, or at least 99% sequence identity to any of SEQ ID NOs: 45-54. In some embodiments, the polynucleotide comprises the sequence of any one of SEQ ID NOs 45-54, provided below. In some embodiments, the polynucleotide comprises the complement of any one of SEQ ID NOs: 45-54. Any of the disclosed vectors may differ relative to any of the following nucleic acid sequences by 1, 2, 3, 4, 5, 10, 10-15, 15-20, 20-30, 30-50, 50-75, 75-100, 100-125, 125-150, 150-175, 175-200, or more than 200 nucleotides. These differences may comprise nucleotides that have been inserted, deleted, or substituted relative to the sequence of any of SEQ ID NOs: 45-54. In some embodiments, the disclosed vectors contain stretches of about 10, about 20, about 25, about 40, about 50, about 75, about 100, about 125, about 150, about 175, or about 180 nucleotides in common with the sequence of any of SEQ ID NOs: 45-54. In some embodiments, the disclosed chimeric receptors comprise truncations at the 5′ or 3′ end relative to any of SEQ ID NOs: 45-54. The signal peptide is indicated single underline; the restriction digestion sites are indicated in italics; and the FLAG tag is indicated in double underline. The “***” notation in the sequences below denotes the position of the DNA insert encoding the scFv extracellular binding domain in a full CPR protein.

>FCERG BACKBONE VECTOR
(SEQ ID NO: 45)
ATGGCCTTACCAGTGACCGCCTTGCTCCTGCCGCTGGCCTTGCTG
CTCCACGCCGCCAGGCCGACGCGT***CTTAAGGAATTCGATTAC
AAGGATGACGACGATAAGACCACGACGCCAGCGCCGCGACCACCA
ACACCGGCGCCCACCATCGCGTCGCAGCCCCTGTCCCTGCGCCCA
GAGGCGTGCCGGCCAGCGGCGGGGGGCGCAGTGCACACGAGGGGG
CTGGACTTCGCCTGTGATATCTACATCTGGGCGCCCTTGGCCGGG
ACTTGTGGGGTCCTTCTCCTGTCACTGGTTATCACCCTTTACTGC
CGACTGAAGATCCAAGTGCGAAAGGCAGCTATAACCAGCTATGAG
AAATCAGATGGTGTTTACACGGGCCTGAGCACCAGGAACCAGGAG
ACTTACGAGACTCTGAAGCATGAGAAACCACCACAGGTT
>MRC1 BACKBONE VECTOR
(SEQ ID NO: 46)
ATGAGGCTACCCCTGCTCCTGGTTTTTGCCTCTGTCATTCCGGGT
GCTGTTCTCCTACTGACGCGT***CTTAAGGAATTCGATTACAA
GGATGACGACGATAAGAGACCAAAAATTATTGATGCTAAACCTAC
TCATGAATTACTTACAACAAAAGCTGACACAAGGAAGATGGACCC
TTCTAAACCGTCTTCCAACGTGGCCGGAGTAGTCATCATTGTGAT
CCTCCTGATTTTAACGGGTGCTGGCCTTGCCGCCTATTTCTTTTA
TAAGAAAAGACGTGTGCACCTACCTCAAGAGGGCGCCTTTGAAAA
CACTCTGTATTTTAACAGTCAGTCAAGCCCAGGAACTAGTGATAT
GAAAGATCTCGTGGGCAATATTGAACAGAATGAACACTCGGTCAT
CGTT
>MerTK BACKBONE VECTOR
(SEQ ID NO: 47)
ATGGGGCCGGCCCCGCTGCCGCTGCTGCTGGGCCTCTTCCTCCCC
GCGCTCTGGCGTAGAGCTATCACGCGT***CTTAAGGAATTCGAT
TACAAGGATGACGACGATAAGGATGCCCCATCAGTAGCACCTTTA
AATGTCACTGTGTTTCTGAATGAATCTAGTGATAATGTGGACATC
AGATGGATGAAGCCTCCGACTAAGCAGCAGGATGGAGAACTGGTG
GGCTACCGGATATCCCACGTGTGGCAGAGTGCAGGGATTTCCAAA
GAGCTCTTGGAGGAAGTTGGCCAGAATGGCAGCCGAGCTCGGATC
TCTGTTCAAGTCCACAATGCTACGTGCACAGTGAGGATTGCAGCC
GTCACCAGAGGGGGAGTTGGGCCCTTCAGTGATCCAGTGAAAATA
TTTATCCCTGCACACGGTTGGGTAGATTATGCCCCCTCTTCAACT
CCGGCGCCTGGCAACGCAGATCCTGTGCTCATCATCTTTGGCTGC
TTTTGTGGATTTATTTTGATTGGGTTGATTTTATACATCTCCTTG
GCCATCAGAAAAAGAGTCCAGGAGACAAAGTTTGGGAATGCATTC
ACAGAGGAGGATTCTGAATTAGTGGTGAATTATATAGCAAAGAAA
TCCTTCTGTCGGCGAGCCATTGAACTTACCTTACATAGCTTGGGA
GTCAGTGAGGAACTACAAAATAAACTAGAAGATGTTGTGATTGAC
AGGAATCTTCTAATTCTTGGAAAAATTCTGGGTGAAGGAGAGTTT
GGGTCTGTAATGGAAGGAAATCTTAAGCAGGAAGATGGGACCTCT
CTGAAAGTGGCAGTGAAGACCATGAAGTTGGACAACTCTTCACAG
CGGGAGATCGAGGAGTTTCTCAGTGAGGCAGCGTGCATGAAAGAC
TTCAGCCACCCAAATGTCATTCGACTTCTAGGTGTGTGTATAGAA
ATGAGCTCTCAAGGCATCCCAAAGCCCATGGTAATTTTACCCTTC
ATGAAATACGGGGACCTGCATACTTACTTACTTTATTCCCGATTG
GAGACAGGACCAAAGCATATTCCTCTGCAGACACTATTGAAGTTC
ATGGTGGATATTGCCCTGGGAATGGAGTATCTGAGCAACAGGAAT
TTTCTTCATCGAGATTTAGCTGCTCGAAACTGCATGTTGCGAGAT
GACATGACTGTCTGTGTTGCGGACTTCGGCCTCTCTAAGAAGATT
TACAGTGGCGATTATTACCGCCAAGGCCGCATTGCTAAGATGCCT
GTTAAATGGATCGCCATAGAAAGTCTTGCAGACCGAGTCTACACA
AGTAAAAGTGATGTGTGGGCATTTGGCGTGACCATGTGGGAAATA
GCTACGCGGGGAATGACTCCCTATCCTGGGGTCCAGAACCATGAG
ATGTATGACTATCTTCTCCATGGCCACAGGTTGAAGCAGCCCGAA
GACTGCCTGGATGAACTGTATGAAATAATGTACTCTTGCTGGAGA
ACCGATCCCTTAGACCGCCCCACCTTTTCAGTATTGAGGCTGCAG
CTAGAAAAACTCTTAGAAAGTTTGCCTGACGTTCGGAACCAAGCA
GACGTTATTTACGTCAATACACAGTTGCTGGAGAGCTCTGAGGGC
CTGGCCCAGGGCTCCACCCTTGCTCCACTGGACTTGAACATCGAC
CCTGACTCTATAATTGCCTCCTGCACTCCCCGCGCTGCCATCAGT
GTGGTCACAGCAGAAGTTCATGACAGCAAACCTCATGAAGGACGG
TACATCCTGAATGGGGGCAGTGAGGAATGGGAAGATCTGACTTCT
GCCCCCTCTGCTGCAGTCACAGCTGAAAAGAACAGTGTTTTACCG
GGGGAGAGACTTGTTAGGAATGGGGTCTCCTGGTCCCATTCGAGC
ATGCTGCCCTTGGGAAGCTCATTGCCCGATGAACTTTTGTTTGCT
GACGACTCCTCAGAAGGCTCAGAAGTCCTGATGGTT
>MEGF10 BACKBONE VECTOR
(SEQ ID NO: 48)
ATGGTTATTTCTTTGAACTCATGCCTGAGCTTTATTTGTTTATTG
TTATGCCACTGGATTACGCGT***CTTAAGGAATTCGATTACAA
GGATGACGACGATAAGAGCCCCGGATGGAAGGGAGCGAGATGTGA
TCAAGCTGGTGTTATCATAGTTGGAAATCTGAACAGCTTAAGCCG
AACCAGTACTGCTCTCCCTGCTGATTCCTACCAGATCGGGGCCAT
TGCAGGCATCATCATTCTTGTCCTAGTTGTTCTCTTCCTACTGGC
ATTGTTCATTATTTATAGACACAAGCAGAAGGGAAAGGAATCAAG
CATGCCAGCAGTTACCTACACCCCTGCTATGAGGGTCGTCAATGC
AGATTATACCATTTCAGGAACCCTTCCTCACAGCAATGGTGGAAA
CGCTAATAGCCACTACTTCACCAATCCCAGTTACCACACGCTCAC
CCAGTGTGCCACATCCCCTCACGTCAACAACAGGGACAGGATGAC
TGTCACGAAGTCAAAAAACAATCAACTGTTTGTGAATCTTAAAAA
TGTGAACCCTGGGAAGAGAGGCCCTGTGGGGGACTGCACTGGGAC
ATTGCCGGCTGACTGGAAACATGGCGGCTACCTCAACGAGCTCGG
TGCTTTTGGACTTGACAGAAGCTATATGGGAAAATCCTTAAAAGA
CCTGGGAAAGAATTCTGAATATAATTCAAGTAACTGCTCCCTAAG
CAGTTCTGAGAACCCATATGCCACTATTAAAGACCCACCTGTACT
TATCCCGAAAAGCTCAGAGTGTGGTTATGTGGAGATGAAATCGCC
GGCACGAAGAGATTCCCCATATGCAGAGATCAATAACTCAACTTC
AGCCAACAGGAATGTCTATGAAGTTGAACCTACAGTGAGTGTTGT
CCAAGGAGTATTCAGCAATAATGGGCGTCTCTCCCAGGATCCATA
TGACCTCCCAAAGAACAGTCACATCCCTTGTCATTATGACCTGCT
GCCAGTCCGAGACAGTTCATCCTCCCCTAAGCAAGAGGACAGTGG
TGGTAGCAGCAGCAACAGCAGCAGCAGCAGTGAAGTT
>DAP12 BACKBONE VECTOR
(SEQ ID NO: 49)
ATGGGGGGACTTGAACCCTGCAGCAGGCTCCTGCTCCTGCCTCTC
CTGCTGGCTGTAAGTGGTCTCCGTACGCGT***CTTAAGGAATTC
GATTACAAGGATGACGACGATAAGCCTGTCCAGGCCCAGGCCCAG
AGCGATTGCAGTTGCTCTACGGTGAGCCCGGGCGTGCTGGCAGGG
ATCGTGATGGGAGACCTGGTGCTGACAGTGCTCATTGCCCTGGCC
GTGTACTTCCTGGGCCGGCTGGTCCCTCGGGGGCGAGGGGCTGCG
GAGGCAGCGACCCGGAAACAGCGTATCACTGAGACCGAGTCGCCT
TATCAGGAGCTCCAGGGTCAGAGGTCGGATGTCTACAGCGACCTC
AACACACAGAGGCCGTATTACAAAGTT
>TREM2 BACKBONE VECTOR
(SEQ ID NO: 50)
ATGGAGCCTCTCCGGCTGCTCATCTTACTCTTTGTCACAGAGCTG
TCCGGAGCCCACACGCGT***CTTAAGGAATTCGATTACAAGGAT
GACGACGATAAGGATCACCGGGATGCTGGAGATCTCTGGTTCCCC
GGGGAGTCTGAGAGCTTCGAGGATGCCCATGTGGAGCACAGCATC
TCCAGGAGCCTCTTGGAAGGAGAAATCCCCTTCCCACCCACTTCC
ATCCTTCTCCTCCTGGCCTGCATCTTTCTCATCAAGATTCTAGCA
GCCAGCGCCCTCTGGGCTGCAGCCTGGCATGGACAGAAGCCAGGG
ACACATCCACCCAGTGAACTGGACTGTGGCCATGACCCAGGGTAT
CAGCTCCAAACTCTGCCAGGGCTGAGAGACACG
>FCERG-MRC1 BACKBONE VECTOR
(SEQ ID NO: 51)
ATGGCCTTACCAGTGACCGCCTTGCTCCTGCCGCTGGCCTTGCTG
CTCCACGCCGCCAGGCCGACGCGT***CTTAAGGAATTCGATTAC
AAGGATGACGACGATAAGACCACGACGCCAGCGCCGCGACCACCA
ACACCGGCGCCCACCATCGCGTCGCAGCCCCTGTCCCTGCGCCCA
GAGGCGTGCCGGCCAGCGGCGGGGGGCGCAGTGCACACGAGGGGG
CTGGACTTCGCCTGTGATATCTACATCTGGGCGCCCTTGGCCGGG
ACTTGTGGGGTCCTTCTCCTGTCACTGGTTATCACCCTTTACTGC
CGACTGAAGATCCAAGTGCGAAAGGCAGCTATAACCAGCTATGAG
AAATCAGATGGTGTTTACACGGGCCTGAGCACCAGGAACCAGGAG
ACTTACGAGACTCTGAAGCATGAGAAACCACCACAGGTTTCGACC
GGTGGCCGTGTGCACCTACCTCAAGAGGGCGCCTTTGAAAACACT
CTGTATTTTAACAGTCAGTCAAGCCCAGGAACTAGTGATATGAAA
GATCTCGTGGGCAATATTGAACAGAATGAACACTCGGTCATCGTT
>MRC1-FCERG BACKBONE VECTOR
(SEQ ID NO: 52)
ATGAGGCTACCCCTGCTCCTGGTTTTTGCCTCTGTCATTCCGGGT
GCTGTTCTCCTACTGACGCGT***CTTAAGGAATTCGATTACAA
GGATGACGACGATAAGAGACCAAAAATTATTGATGCTAAACCTAC
TCATGAATTACTTACAACAAAAGCTGACACAAGGAAGATGGACCC
TTCTAAACCGTCTTCCAACGTGGCCGGAGTAGTCATCATTGTGAT
CCTCCTGATTTTAACGGGTGCTGGCCTTGCCGCCTATTTCTTTTA
TAAGAAAAGACGTGTGCACCTACCTCAAGAGGGCGCCTTTGAAAA
CACTCTGTATTTTAACAGTCAGTCAAGCCCAGGAACTAGTGATAT
GAAAGATCTCGTGGGCAATATTGAACAGAATGAACACTCGGTCAT
CGTTTCGACCGGTGGCCGACTGAAGATCCAAGTGCGAAAGGCAGC
TATAACCAGCTATGAGAAATCAGATGGTGTTTACACGGGCCTGAG
CACCAGGAACCAGGAGACTTACGAGACTCTGAAGCATGAGAAACC
ACCACAGGTT
>M6PR BACKBONE VECTOR
(SEQ ID NO: 53)
ATGTTCCCTTTCTACAGCTGCTGGAGGACTGGACTGCTACTACTA
CTCCTGGCTGTGGCAGTGAGAGAATCCTGGCAGACAACGCGT***
CTTAAGGAATTCGATTACAAGGATGACGACGATAAGGGTGATGAA
TATGACAACCACTGTGGCAAGGAGCAGCGTCGTGCAGTGGTGATG
ATCTCCTGCAATCGACACACCCTAGCGGACAATTTTAACCCTGTG
TCTGAGGAGCGTGGCAAAGTCCAAGATTGTTTCTACCTCTTTGAG
ATGGATAGCAGCCTGGCCTGTTCACCAGAGATCTCCCACCTCAGT
GTGGGTTCCATCTTACTTGTCACGTTTGCATCACTGGTTGCTGTT
TATGTTGTTGGGGGGTTCCTATACCAGCGACTGGTAGTGGGAGCC
AAAGGAATGGAGCAGTTTCCCCACTTAGCCTTCTGGCAGGATCTT
GGCAACCTGGTAGCAGATGGCTGTGACTTTGTCTGCCGTTCTAAA
CCTCGAAATGTGCCTGCAGCATATCGTGGTGTGGGGGATGACCAG
CTGGGGGAGGAGTCAGAAGAAAGGGATGACCATTTATTACCAATG
GTT
>CLEC4L BACKBONE VECTOR
(SEQ ID NO: 54)
ATGAGTGACTCCAAGGAACCAAGACTGCAGCAGCTGGGCCTCCTG
GAGGAGGAACAGCTGAGAGGCCTTGGATTCCGACAGACTCGGGGA
TACAAGAGCTTAGCAGGGTGTCTTGGCCATGGTCCCCTGGTGCTG
CAACTCCTCTCCTTCACGCTCTTGGCTGGGCTCCTTGTCCAAGTG
TCCAAGGTCCCCAGCTCCATAAGTCAGGAACAATCCAGGCAAGAC
GCGATCTACCAGAACCTGACCCAGCTTAAAGCTGCAGTGGGTGAG
CTCTCAGAGAAATCCAAGCTGCAGGAGATCTACCAGGAGCTGACC
CAGCTGAAGGCTGCAGTGGGTGAGCTTCCAGAGAAATCTAAGCTG
CAGGAGATCTACCAGGAGCTGACCCGGCTGAAGGCTGCAGTGGGT
GAGCTTCCAGAGAAATCTAAGCTGCAGGAGATCTACCAGGAGCTG
ACCTGGCTGAAGGCTGCAGTGGGTGAGCTTCCAGAGAAATCTAAG
ATGCAGGAGATCTACCAGGAGCTGACTCGGCTGAAGGCTGCAGTG
GGTGAGCTTCCAGAGAAATCTAAGCAGCAGGAGATCTACCAGGAG
CTGACCCGGCTGAAGGCTGCAGTGGGTGAGCTTCCAGAGAAATCT
AAGCAGCAGGAGATCTACCAGGAGCTGACCCGGCTGAAGGCTGCA
GTGGGTGAGCTTCCAGAGAAATCTAAGCAGCAGGAGATCTACCAG
GAGCTGACCCAGCTGAAGGCTGCAGTGGAACGCCTGTGCCACCCC
TGTCCCTGGGAATGGACAGATTACAAGGATGACGACGATAAGGGT
GGCGGCGGTAGCGGTGGTGGCGGTAGCGGCGGTGGTGGCAGCTTG
GTACCGAGCTCGGATCCAGGTCACCTGGTGGAATTC***GGAGGA
AGCCATCATCATCATCATCATTGA

### Linkers

In various embodiments of the disclosed chimeric receptors, linker domains may be used to link any of the peptide domains, such as the EBD and TMD, and/or two or more ICDs to one another. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

The length and amino acid composition of the linker domain can be optimized to vary the orientation and/or proximity of the ICD, EBD, TMD, and/or other domains to one another to achieve a desired activity of the chimeric polypeptide. In some embodiments, the orientation and/or proximity of these domains to one another can be varied to enhance or reduce the biological activity of the chimeric polypeptide.

In some embodiments, the linker is an amino acid or a plurality of amino acids (e.g., a peptide or protein). In some embodiments, the linker is a bond (e.g., a covalent bond), an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35-40, 40-45, 45-50, 50-60, 60-70, 70-80, 80-90, 90-100, 100-110, 110-120, 120-130, 130-140, 140-150, or 150-200 amino acids in length. Longer or shorter linkers are also contemplated. In some embodiments, the linker comprises the amino acid sequence GGGS (SEQ ID NO: 55) or GGGSGGGS (SEQ ID NO: 56).

### Pharmaceutical Compositions and Methods of Treatment

Provided herein are pharmaceutical compositions that comprise a modified cell as disclosed herein. These modified cells may comprise astrocytes, microglial cells, or monocytes. In exemplary embodiments, provided herein are pharmaceutical compositions comprising modified astrocytes. These modified cells may be contacted ex vivo with a nucleic acid or expression vector expressing one of the disclosed CPRs. In some embodiments, the expression vector is a viral vector, such as an rAAV vector or a lentiviral vector. In particular embodiments, the expression vector is an rAAV vector.

In some aspects, ex vivo delivery of cells transduced with rAAV particles or preparations is provided herein. Ex vivo gene delivery may be used to transplant rAAV-transduced host cells that were isolated from the host back into the host. A suitable ex vivo protocol may include several steps. For example, a segment of target tissue or an aliquot of target fluid may be harvested from the host and rAAV particles or preparations may be used to transduce a polynucleotide into the host cells in the tissue or fluid. These genetically modified cells may then be transplanted back into the host. Several approaches may be used for the reintroduction of cells into the host, including intravenous injection, ICV injection, or in situ injection into target tissue. Autologous and allogeneic cell transplantation may be used according to the disclosure.

An effective amount of cells in a pharmaceutical composition for ex vivo delivery to a subject is at least one cell (for example, one modified astrocyte cell), or is more typically greater than 100 cells, for example, up to 10⁶, up to 10⁷, up to 10⁸ cells, up to 10⁹ cells, up to 10¹⁰ cells, or up to 10¹¹ cells or more. In certain embodiments, the cells are administered in a range from about 10⁶ to about 10¹⁰ cells/m². The number of cells will depend upon the ultimate use for which the composition is intended, as well the type of cells included therein. For example, a composition comprising cells modified to contain a CPR specific for a particular neurodegenerative disease antigen (e.g., amyloid beta) will comprise a cell population containing from about 5% to about 95% or more of such cells. In certain embodiments, a composition comprising CPR modified cells comprises a cell population comprising at least 5%, 10%, 15%, 20%, 25%, 30%, 35%, 40%, 45%, 50%, 55%, 60%, 65%, 70%, 75%, 80%, 85%, 90%, 95% or more of such cells.

Further provided herein are pharmaceutical compositions that comprise a viral vector as disclosed herein, and further comprise a pharmaceutical excipient, and may be formulated for administration to host cell ex vivo or in situ in an animal, and particularly a human. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Such compositions may be formulated for use in a variety of therapies, such as for example, in the amelioration, prevention, and/or treatment of conditions such as peptide deficiency, polypeptide deficiency, peptide overexpression, polypeptide overexpression, including for example, conditions, diseases or disorders as described herein.

The term “excipient” refers to a diluent, adjuvant, carrier, or vehicle with which the rAAV particle or preparation, or composition of modified cells is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers.

In certain embodiments, the present disclosure provides a method of reducing AAV immunity in a subject, wherein the method further comprises administering to the subject a composition comprising the disclosed rAAV particles and a pharmaceutically acceptable excipient, optionally wherein the subject has been previously administered a composition comprising rAAV particles. In particular embodiments, the subject is a human.

In some embodiments, the number of rAAV particles administered to a host cell may be on the order ranging from 500 to 5,000 vector genomes (vgs)/cell. In particular embodiments, the disclosed methods comprise administration of rAAV particles in doses of about 500 vgs/cell, 1000 vgs/cell, 2000 vgs/cell, 3000 vgs/cell, 4000 vgs/cell, 5000 vgs/cell, 6000 vgs/cell or 7000 vgs/cell.

In some embodiments, the number of rAAV particles administered to a subject may be on the order ranging from 10⁶ to 10¹⁴ particles/mL or 10³ to 10¹³ particles/mL, or any values therebetween for either range, such as for example, about 10⁶, 10⁷, 10⁸, 10⁹, 10¹⁰, 10¹¹, 10¹², 10¹³, or 10¹⁴ particles/mL. In one embodiment, rAAV particles of higher than 10¹³ particles/mL are be administered. In some embodiments, the number of rAAV particles administered to a subject may be on the order ranging from 10⁶ to 10¹⁴ vgs/mL or 10³ to 10¹⁵ vgs/mL, or any values there between for either range, such as for example, about 10⁶, 10⁷, 10⁸, 10⁹, 10¹⁰, 10¹¹, 10¹², 10¹³, or 10¹⁴ vgs/mL. In certain embodiments, the disclosed methods comprise administration of rAAV particle compositions in doses of 1×10¹⁰-4×10¹⁰ vgs/mL. In certain embodiments, the disclosed methods comprise administration of rAAV particle compositions in doses of 1×10¹⁰-4×10¹⁰ vgs per animal subject. In some embodiments, rAAV particles of higher than 10¹³ vgs/mL are be administered.

The rAAV particles can be administered as a single dose, or divided into two or more administrations as may be required to achieve therapy of the particular disease or disorder being treated. In some embodiments, 0.0001 mL to 10 mL are delivered to a subject.

In some embodiments, the disclosure provides formulations of compositions disclosed herein in pharmaceutically acceptable solutions for administration to a cell or an animal, either alone or in combination with one or more other modalities of therapy, and in particular, for therapy of human cells, tissues, and diseases affecting man.

If desired, rAAV particle or preparation and compositions may be administered in combination with other agents as well, such as, e.g., proteins or polypeptides or various pharmaceutically-active agents, including one or more systemic or topical administrations of therapeutic polypeptides, biologically active fragments, or variants thereof. In fact, there is virtually no limit to other components that may also be included, given that the additional agents do not cause a significant adverse effect upon contact with the target cells or host tissues. The rAAV particles or preparations and compositions of modified cells may thus be delivered along with various other agents as required in the particular instance. Such compositions may be purified from host cells or other biological sources, or alternatively may be chemically synthesized as described herein.

Formulation of pharmaceutically-acceptable excipients is well-known to those of skill in the art, as is the development of suitable dosing and treatment regimens for using the particular compositions described herein in a variety of treatment regimens, including e.g., oral, parenteral, intravenous, intranasal, intra-articular, and intramuscular administration and formulation.

Typically, these formulations may contain at least about 0.1% of the therapeutic agent (e.g., rAAV particle or preparation and/or composition of modified cells) or more, although the percentage of the active ingredient(s) may, of course, be varied and may conveniently be between about 1 or 2% and about 70% or 80% or more of the weight or volume of the total formulation. Naturally, the amount of therapeutic agent(s) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it is desirable to deliver the rAAV particles or preparations and/or compositions of modified cells in suitably formulated pharmaceutical compositions disclosed herein either subcutaneously, intraocularly, intravitreally, parenterally, subcutaneously, intravenously, intracerebro-ventricularly, intraocularly, intramuscularly, intrathecally, orally, intraperitoneally, by oral or nasal inhalation, or by direct injection to one or more cells, tissues, or organs by direct injection. In particular embodiments, the disclosed rAAV particle compositions and compositions of modified cells are formulated for intraocular, ICV, or IV delivery.

The pharmaceutical forms of the compositions suitable for injectable use include sterile aqueous solutions or dispersions. In some embodiments, the form is sterile and fluid to the extent that easy syringability exists. In some embodiments, the form is stable under the conditions of manufacture and storage and is preserved against the contaminating action of microorganisms, such as bacteria and fungi. The carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

The pharmaceutical compositions of the present disclosure can be administered to the subject being treated by standard routes including, but not limited to, pulmonary, intranasal, oral, inhalation, parenteral such as intravenous, intraocular, ICV, topical, transdermal, intradermal, transmucosal, intraperitoneal, intramuscular, intracapsular, intraorbital, intravitreal, intracardiac, transtracheal, subcutaneous, subcuticular, intraarticular, subcapsular, subarachnoid, intraspinal, epidural and intrasternal injection.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, intravitreal, subcutaneous and intraperitoneal administration. In this connection, a sterile aqueous medium that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

Sterile injectable solutions are prepared by incorporating the rAAV particles or preparations and/or compositions of modified cells, in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The amount of rAAV particle or preparation or modified cell compositions and time of administration of such compositions will be within the purview of the skilled artisan having benefit of the present teachings. It is likely, however, that the administration of therapeutically-effective amounts of the disclosed compositions may be achieved by a single administration, such as for example, a single injection of sufficient numbers of infectious particles to provide therapeutic benefit to the patient undergoing such treatment. Alternatively, in some circumstances, it may be desirable to provide multiple, or successive administrations of the rAAV particle or preparation or composition, either over a relatively short, or a relatively prolonged period of time, as may be determined by the medical practitioner overseeing the administration of such compositions.

The composition may include rAAV particles or preparations or modified cell compositions, either alone, or in combination with one or more additional active ingredients, which may be obtained from natural or recombinant sources or chemically synthesized. In some embodiments, rAAV particles or preparations are administered in combination, either in the same composition or administered as part of the same treatment regimen, with a proteasome inhibitor, such as Bortezomib, or hydroxyurea.

In other aspects, provided herein are methods of treatment comprising administration of a chimeric receptor as described herein to a subject in need thereof. In certain embodiments, methods of treatment comprise administering a pharmaceutical composition, rAAV particle, or nanoparticle as described herein.

To “treat” a disease as the term is used herein, means to reduce the frequency or severity of at least one sign or symptom of a disease or disorder experienced by a subject. The compositions described above are typically administered to a subject in an effective amount, that is, an amount capable of producing a desirable result. The desirable result will depend upon the active agent being administered. For example, an effective amount of a rAAV particle may be an amount of the particle that is capable of transferring a heterologous nucleic acid to a host organ, tissue, or cell.

In certain embodiments, provided herein are methods of treating a subject having or at risk of developing a disease, disorder, or condition comprising administering to the subject a chimeric receptor or modified cell comprising a modified receptor as described herein. In other embodiments, the methods of treatment of a subject having or at risk of developing a disease, disorder, or condition comprise administering one or more pharmaceutical compositions, nanoparticles, or rAAV particles described herein.

In some embodiments, the subject has been diagnosed with a disease, disorder, or condition. In some embodiments, the subject has been diagnosed with an amyloid-related disease or disorder. In particular embodiments, the amyloid-related disease or disorder is Alzheimer's Disease, Parkinson's Disease, or another neurodegenerative disorder.

Toxicity and efficacy of the compositions utilized in methods of the disclosure can be determined by standard pharmaceutical procedures, using either cells in culture or experimental animals to determine the LD₅₀ (the dose lethal to 50% of the population). The dose ratio between toxicity and efficacy the therapeutic index and it can be expressed as the ratio LD₅₀/ED₅₀. Those compositions that exhibit large therapeutic indices are preferred. While those that exhibit a tolerable level of toxic side effects may be used, care should be taken to design a delivery system that minimizes the potential damage of such side effects. The dosage of compositions as described herein lies generally within a range that includes an ED₅₀ with little or no toxicity. The dosage may vary within this range depending upon the dosage form employed and the route of administration utilized.

Recombinant AAV (rAAV) Particles and Genomes

Aspects of the disclosure relate to recombinant adeno-associated virus (rAAV) particles or preparations of such particles for delivery of one or more polynucleotides or vectors comprising a sequence encoding a heterologous peptide, into various tissues, organs, and/or cells. In some embodiments, the rAAV particle is delivered to a host cell as described herein.

The wild-type AAV genome is a single-stranded deoxyribonucleic acid (ssDNA), either positive- or negative-sensed. The genome comprises two inverted terminal repeats (ITRs), one at each end of the DNA strand, two open reading frames (ORFs): rep and cap between the ITRs, and an insert nucleic acid positioned between the ITRs and optionally comprising a transgene. The rep ORF comprises four overlapping genes encoding Rep proteins required for the AAV life cycle. The cap ORF comprises overlapping genes encoding capsid proteins: VP1, VP2 and VP3, which interact together to form the viral capsid. VP1, VP2 and VP3 are translated from one mRNA transcript, which can be spliced in two different manners: either a longer or shorter intron can be excised resulting in the formation of two isoforms of mRNAs: a ˜2.3 kb- and a ˜2.6 kb-long mRNA isoform. The capsid forms a supramolecular assembly of approximately 60 individual capsid protein subunits into a non-enveloped, T-1 icosahedral lattice capable of protecting the AAV genome. The mature capsid is composed of VP1, VP2, and VP3 (molecular masses of approximately 87, 73, and 62 kDa respectively) in a ratio of about 1:1:10.

Recombinant AAV (rAAV) particles may comprise a nucleic acid segment, which may comprise at a minimum: (a) one or more transgenes comprising a sequence encoding a heterologous peptide or an RNA of interest (e.g., a siRNA or microRNA) and (b) one or more regions comprising inverted terminal repeat (ITR) sequences (e.g., engineered ITR sequences) flanking the one or more heterologous nucleic acid regions (e.g., transgenes). In some embodiments, the ITRs are of the AAV2 serotype. In some embodiments, the nucleic acid segment is between 4 kb and 5 kb in size (e.g., 4.2 to 4.7 kb in size).

Any nucleic acid segment described herein may be encapsidated by a viral capsid, such as an AAV6 capsid or another serotype (e.g., a serotype that is of the same serotype as the ITR sequences), which may comprises a modified capsid protein as described herein. In some embodiments, the rAAV vector is pseudotyped with AAV2 ITRs and a modified AAV6 capsid (i.e., an rAAV2/6 vector or a variant thereof). In some embodiments, the modified capsid of any of the disclosed rAAV particles is an PHP.eB capsid. In some embodiments, the modified capsid is an rAAV6(Y705F+Y731F+T492V) capsid (also known as a TM6 or AAV6-3pMut capsid). In some embodiments, the modified capsid is an rAAV2(Y444F+Y500F+Y730F) capsid (also known as a TM2, trpYF, or AAV2 triple mutant capsid). In some embodiments, provided herein are rAAV particles comprising any of the disclosed vectors, for instance a vector containing an GFAP promoter, and further comprising a PHP.eB capsid. In some embodiments, provided herein are rAAV particles comprising any of the disclosed vectors, for instance a vector containing a CD8 promoter, and further comprising a TM6 capsid.

In some embodiments, the nucleic acid segment of the rAAV vector is circular. In some embodiments, the nucleic acid segment is single-stranded. In some embodiments, the nucleic acid segment is double-stranded. In some embodiments, a double-stranded nucleic acid segment may be, for example, a self-complimentary vector that contains a region of the nucleic acid segment that is complementary to another region of the nucleic acid segment, initiating the formation of the double-strandedness of the nucleic acid segment.

Accordingly, in some embodiments, an rAAV particle or rAAV preparation containing such particles comprises a viral capsid and a nucleic acid segment as described herein, which is encapsidated by the viral capsid. In some embodiments, the insert nucleic acid of the nucleic acid segment comprises (1) one or more transgenes comprising a sequence encoding a heterologous peptide, (2) one or more nucleic acid regions comprising a sequence that facilitates expression of the transgene (e.g., a promoter), and (3) one or more nucleic acid regions comprising a sequence that facilitate integration of the transgene (optionally with the one or more nucleic acid regions comprising a sequence that facilitates expression) into the genome of the subject. In certain embodiments, the promoter of the insert nucleic acid comprises a sequence that has at least 90%, at least 95%, or at least 99% identity to a chicken β-actin (CBA) promoter.

In some embodiments, the polynucleotides and vectors described herein comprise ITR sequences. In some embodiments, the coding sequence and associated promoter are flanked by rAAV ITR sequences. The ITR sequences of a polynucleotide described herein can be derived from any AAV serotype (e.g., 1, 2, 3, 4, 5, 6, 7, 8, 9, or 10) or can be derived from more than one serotype. In some embodiments, the ITR sequences are derived from AAV2 or AAV6. In some embodiments, the ITR sequences of the first serotype are derived from AAV3, AAV2 or AAV6. In other embodiments, the ITR sequences of the first serotype are derived from AAV1, AAV5, AAV8, AAV9 or AAV10. In some embodiments, the ITR sequences are the same serotype as the capsid (e.g., AAV3 ITR sequences and AAV3 capsid, etc.).

ITR sequences and plasmids containing ITR sequences are known in the art and commercially available (see, e.g., products and services available from Vector Biolabs, Philadelphia, PA; Cellbiolabs, San Diego, CA; Agilent Technologies, Santa Clara, Ca; and Addgene, Cambridge, MA; and Gene delivery to skeletal muscle results in sustained expression and systemic delivery of a therapeutic protein. Kessler P D, et al. Proc Natl Acad Sci USA. 1996; 93(24):14082-7; and Curtis A. Machida, Methods in Molecular Medicine™ Viral Vectors for Gene Therapy Methods and Protocols. 10.1385/1-59259-304-6:201 Humana Press Inc. 2003: Chapter 10, Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

In some embodiments, the nucleic acid segment comprises a pTR-UF-11 plasmid backbone, which is a plasmid that contains AAV2 ITRs. This plasmid is commercially available from the American Type Culture Collection (ATCC MBA-331).

The rAAV particle comprising a nucleic acid segment (in any form contemplated herein) may be delivered in the form of a composition, such as a composition comprising the active ingredient, such as the rAAV particle, nucleic acid segment (in any form contemplated herein), and a therapeutically or pharmaceutically acceptable carrier. The rAAV particles or nucleic acid segment may be prepared in a variety of compositions, and may also be formulated in appropriate pharmaceutical vehicles for administration to human or animal subjects.

Other aspects of the disclosure are directed to methods that involve contacting cells with an rAAV preparation produced by a method described herein. The contacting may be, e.g., ex vivo or in vivo by administering the rAAV preparation to a subject. The rAAV particle or preparation may be delivered in the form of a composition, such as a composition comprising the active ingredient, such as a rAAV particle or preparation described herein, and a therapeutically or pharmaceutically acceptable excipient. The rAAV particles or preparations may be prepared in a variety of compositions, and may also be formulated in appropriate pharmaceutical vehicles for administration to human or animal subjects.

The rAAV particle or particle within an rAAV preparation may be of any AAV serotype, including any derivative or pseudotype (e.g., 1, 2, 3, 4, 5, 6, 7, 8, 9, 10, 2/1, 2/5, 2/6, 2/8, 2/9, 3/1, 3/5, 3/8, or 3/9). As used herein, the serotype of an rAAV viral vector (e.g., an rAAV particle) refers to the serotype of the capsid proteins of the recombinant virus. In some embodiments, the rAAV particle is not AAV2. In some embodiments, the rAAV particle is not AAV8. Non-limiting examples of derivatives and pseudotypes include rAAV.PHP.eB, AAV6(TM6), AAV2(Y444F+Y500F+Y730F) (i.e., TM2), rAAV2/1, rAAV2/5, rAAV2/6, rAAV2/8, rAAV2/9, AAV2-AAV3 hybrid, AAVrh.10, AAVrh.74, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2(Y4F), AAV8 (Y733F), AAV2.15, AAV2.4, AAVM41, AAV-DJ, and AAVr3.45. The AAV6(TM6) (also known as AAV6-3pmut and AAV6(Y705F+Y731F+T492V)) capsid is described in Rosario et al., Microglia-specific targeting by novel capsid-modified AAV6 vectors, Mol Ther Methods Clin Dev. 2016; 13; 3:16026 and International Patent Publication No. PCT/US 2016/126857, each of which are herein incorporated by reference.

These AAV serotypes and derivatives/pseudotypes, and methods of producing such derivatives/pseudotypes are known in the art (see, e.g., Mol Ther. 2012 April; 20(4):699-708). The AAV vector toolkit: poised at the clinical crossroads. Asokan A1, Schaffer D V, Samulski R J.). In some embodiments, the rAAV particle is a pseudotyped rAAV particle, which comprises (a) a nucleic acid segment comprising ITRs from one serotype (e.g., AAV2, AAV3) and (b) a capsid comprised of capsid proteins derived from another serotype (e.g., AAV1, AAV2, AAV3, AAV4, AAV5, AAV6, AAV7, AAV8, AAV9, or AAV10). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Additional capsids suitable for use in the disclosed rAAV particles include the following: capsids comprising non-native amino acid substitutions at amino acid residues of a wild-type AAV2 capsid, wherein the non-native amino acid substitutions comprise one or more of Y272F, Y444F, T491V, Y500F, Y700F, Y704F and Y730F; and capsids comprising non-native amino acid substitutions at amino acid residues of a wild-type AAV6 capsid, wherein the non-native amino acid substitutions comprise one or more of Y445F, Y705F, Y731F, T492V and S663V.

Additional serotypes of the rAAV capsids disclosed herein include capsids include AAV7m8, AAV2/2-MAX, AAVSHh10Y, AAV3b, AAVLK03, AAV7BP2, AAV1(E531K), AAV6(D532N), and AAV2G9.

The AAV-DJ capsid is described in Grimm et al., J. Virol., 2008, 5887-5911 and Katada et al., (2019) Evaluation of AAV-DJ vector for retinal gene therapy, PeerJ 7:e6317 each of which is herein incorporated by reference. The AAV-DJ comprises the insertion of 7 amino acids into the HSPG binding domain of the AAV2 capsid and has high expression efficiency in Muller cells following intravitreal injection. The AAV7m8 capsid, which is closely related to AAV-DJ, is described in Dalkara et al. Sci Transl Med. 2013; 5(189):189ra76, herein incorporated by reference.

The AAV2/2-MAX capsid is described in Reid, Ertel & Lipinski, Improvement of Photoreceptor Targeting via Intravitreal Delivery in Mouse and Human Retina Using Combinatory rAAV2 Capsid Mutant Vectors, Invest. Ophthalmol Vis Sci. 2017; 58:6429-6439, herein incorporated by reference. The AAV2/2-MAX capsid comprises five point mutations, Y272F, Y444F, Y500F, Y730F, T491V. The AAV1(E531K) capsid is described in Boye et al., Impact of Heparan Sulfate Binding on Transduction of Retina by Recombinant Adeno-Associated Virus Vectors, J. Virol. 90:4215-4231 (2016), herein incorporated by reference.

The AAVSHh10 and AAV6(D532N) capsids, both derivatives of AAV6, are described in Klimczak et al. (2009) A Novel Adeno-Associated Viral Variant for Efficient and Selective Intravitreal Transduction of Rat Muller Cells. PLoS ONE 4(10): e746, herein incorporated by reference.

### Production Methods

Methods of producing rAAV particles are described herein. Other methods are also known in the art and commercially available (see, e.g., Zolotukhin et al. Production and purification of serotype 1, 2, and 5 recombinant adeno-associated viral vectors. Methods 28 (2002) 158-167; and U.S. Patent Publication Nos. US 2007/0015238 and US 2012/0322861, which are incorporated herein by reference; and plasmids and kits available from ATCC and Cell Biolabs, Inc.). For example, a plasmid containing the nucleic acid segment may be combined with one or more helper plasmids, e.g., that contain a rep gene (e.g., encoding Rep78, Rep68, Rep52 and Rep40) and a cap gene (encoding VP1, VP2, and VP3, including a modified VP3 region as described herein), and transfected into a producer cell line such that the rAAV particle can be packaged and subsequently purified.

In some embodiments, the one or more helper plasmids include a first helper plasmid comprising a rep gene and a cap gene and a second helper plasmid comprising a Ela gene, a E1b gene, a E4 gene, a E2a gene, and a VA gene. In some embodiments, the rep gene is a rep gene derived from AAV3, AAV5, or AAV6 and the cap gene is derived from AAV2, AAV3, AAV5, or AAV6 and may include modifications to the gene in order to produce the modified capsid protein described herein. In some embodiments, the rep gene is a rep gene derived from AAV1 or AAV2 and the cap gene is derived from AAV1 or AAV2 and may include modifications to the gene in order to produce the modified capsid protein described herein. Helper plasmids, and methods of making such plasmids, are known in the art and commercially available (see, e.g., pDM, pDG, pDP1rs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, PA; Cellbiolabs, San Diego, CA; Agilent Technologies, Santa Clara, Ca; and Addgene, Cambridge, MA; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081.; Grimm et al. (2003), Helper Virus-Free, Optically Controllable, and Two-Plasmid-Based Production of Adeno-associated Virus Vectors of Serotypes 1 to 6, Molecular Therapy,Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R.O. (2008), International efforts for recombinant adeno-associated viral vector reference standards, Molecular Therapy, Vol. 16, 1185-1188).

An exemplary, non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. In some embodiments, the one or more helper plasmids comprise rep genes for a first serotype (e.g., AAV3, AAV5, and AAV6), cap genes (which may or may not be of the first serotype) and optionally one or more of the adenoviral VA, E2A (DBP), and E4 genes under the transcriptional control of their native promoters. In some embodiments, the one or more helper plasmids comprise cap ORFs (and optionally rep ORFs) for the desired AAV serotype and the adenoviral VA, E2A (DBP), and E4 genes under the transcriptional control of their native promoters. The cap ORF may also comprise one or more modifications to produce a modified capsid protein as described herein. HEK293 cells (available from ATCC®) are transfected via CaPO4-mediated transfection, lipids or polymeric molecules such as Polyethylenimine (PEI) with the helper plasmid(s) and a plasmid containing a nucleic acid segment described herein. The HEK293 cells are then incubated for at least 60 hours to allow for rAAV particle production. Alternatively, in another example Sf9-based producer stable cell lines are infected with a single recombinant baculovirus containing the nucleic acid segment. As a further alternative, in another example HEK293 or BHK cell lines are infected with a HSV containing the nucleic acid segment and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known the art or described herein, e.g., by iodixanol step gradient, CsCl gradient, chromatography, or polyethylene glycol (PEG) precipitation.

### Host Cells

The disclosure also contemplates modified host cells that comprise at least one of the disclosed rAAV particles or expression vectors described herein. Such host cells include mammalian host cells, with human host cells being preferred, and may be either isolated, in cell or tissue culture. In the case of genetically modified animal models (e.g., a mouse), the transformed host cells may be comprised within the body of the animal itself.

The disclosure also contemplates host cells that comprise at least one of the disclosed rAAV particles, polynucleotides or vectors. Such host cells include mammalian host cells, with human host cells being preferred, and may be either isolated, in cell or tissue culture. In the case of genetically modified animal models (e.g., a mouse), the transformed host cells may be comprised within the body of a non-human animal itself.

In some embodiments, the modified host cells are astrocytes. In some embodiments, the host cells are microglial cells. In some embodiments, the host cell is a monocyte-like (or macrophage-like) RAW264.7 cell. In some embodiments, the host cell is a cancer cell, such as an HEK293 cell or an H4 (human neuroglioma) cell. In some embodiments, the host cell is a human stem cell, such as a human pluripotent stem cell. In some embodiments, the host cell is an induced pluripotent stem cell (iPSC). In particular embodiments, the host cell is an iPSC that can be (or has been) differentiated into a microglial cell or an astrocyte cell.

In some embodiments, a host cell as described herein is derived from a subject as described herein. Host cells may be derived using any method known in the art, e.g., by isolating cells from a fluid or tissue of the subject. In some embodiments, the host cells are cultured. Methods for isolating and culturing cells are well known in the art. In some embodiments, cells having phagocytic activity are used in the compositions and methods described herein. A source of phagocytic cells, such as monocytes, is obtained from a subject. Non-limiting examples of subjects include humans, dogs, cats, mice, rats, and transgenic species thereof. Preferably, the subject is a human. The cells can be obtained from a number of sources, including peripheral blood mononuclear cells, bone marrow, lymph node tissue, spleen tissue, umbilical cord, and tumors. In certain embodiments, any number of monocyte or progenitor cell lines available in the art, may be used. In certain embodiments, the cells can be obtained from a unit of blood collected from a subject using any number of techniques known to the skilled artisan. In some embodiments, cells from the circulating blood of an individual are obtained by apheresis or leukapheresis.

In some embodiments, a population or plurality of cells comprises the monocytes, astrocytes, or microglial cells of the present disclosure. Examples of a population of cells include, but are not limited to, a purified population of microglia, monocytes or astrocytes, and a cell line. In another embodiment, peripheral blood mononuclear cells comprise the population of monocytes. In yet another embodiment, purified cells comprise the population of monocytes.

In some embodiments, the cells or population of cells comprising monocytes, astrocytes, or microglia are cultured for expansion. In another embodiment, the cells or population of cells comprising progenitor cells are cultured for differentiation and expansion of monocytes, macrophages, or dendritic cells. The present disclosure comprises expanding a population of monocytes, macrophages, or dendritic cells comprising a CPR described herein. Specific cell subsets can be collected in accordance with known techniques and enriched or depleted by known techniques, such as affinity binding to antibodies, flow cytometry and/or immunomagnetic selection. Aβ.er enrichment steps and introduction of a CPR, in vitro expansion of the desired modified cells can be carried out in accordance with known techniques, or variations thereof that will be apparent those skilled in the art.

In some aspects, the present disclosure provides methods for enhancing the phagocytic activity of a cell comprising introducing into a host cell a polynucleotide encoding a CPR or a vector encoding a CPR according to any of the embodiments described herein; and expressing the CPR in the host cell, wherein the at least one CPR is specific to a neurodegenerative disease antigen that is naturally targeted by the host cell and expression of the at least one CPR by the host cell enhances the phagocytosis by the cell of a target peptide containing the neurodegenerative disease antigen. In certain embodiments, the target peptide is misfolded, fibrillized, or aggregated. In certain embodiments, the phagocytic activity of a modified cell is enhanced at least about 25%, 30%, 35%, 40%, 50%, 55%, 60%, 65%, 70%, 75%, 80%, 85%, 90%, 95%, 100%, 110%, 120%, 130%, 140%, 150%, 160%, 170%, 180%, 190%, 200% or more as compared to host cell that is not modified.

Delivery Methods Other than rAAV

In some aspects, the present disclosure provides methods of delivering one or more purified proteins, such as the chimeric receptors described herein, or one or more polynucleotides or vectors encoding these chimeric receptors, to a subject or a host cell or tissue. In some embodiments, the method of delivery provided comprises nanoparticle delivery, nucleofection, microinjection, biolistics, virosomes, liposomes, immunoliposomes, polycation or cationic lipid conjugates, naked DNA, artificial virions, and agent-enhanced uptake of DNA. In some embodiments, a nanoparticle comprising any of the disclosed CPRs, polynucleotides, vectors, or pharmaceutical compositions is administered to a subject in need thereof.

Exemplary methods of delivery of polynucleotides and expression vectors include cationic polymer:DNA complexes (e.g. using polyethylenimine (PEI)), lipofection, nucleofection, phosphoration, stable genome integration (e.g., piggybac), microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™, Lipofectin™ and SF Cell Line 4D-Nucleofector X Kit™ (Lonza)). Cationic and neutral lipids that are suitable for efficient receptor-recognition lipofection of polynucleotides include those of Feigner, WO 1991/17424 and WO 1991/16024. Delivery may be to cells (e.g. in vitro or ex vivo administration) or target tissues (e.g. in vivo administration).

Exemplary methods of delivery of purified chimeric receptors include nanoparticle and lipid particles, such as cationic lipid:peptide conjugates. Exemplary nanoparticles may be protein-based or polymer-based and are known in the art.

### Subjects

Aspects of the disclosure relate to methods and preparations for use with a subject, such as human or non-human primate subjects, a host cell in situ in a subject, or a host cell derived from a subject. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, the subject is a human subject. Other exemplary subjects include domesticated animals such as dogs and cats; livestock such as horses, cattle, pigs, sheep, goats, and chickens; and other animals such as mice, rats, guinea pigs, and hamsters.

In some embodiments, the subject has or is suspected of having a disease that may be treated with gene therapy. In particular embodiments, the disease is a neurodegenerative disease. In particular embodiments, the disease is AD or PD.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Antibody Fragments

It may be preferable to use fragments of human antibodies when generating the antigen binding domain (scFv, or EBD) of the disclosed CPRs. Completely human antibodies are particularly desirable for therapeutic treatment of human subjects. Human antibodies can be made by a variety of methods known in the art including phage display methods using antibody libraries derived from human immunoglobulin sequences, including improvements to these techniques. See U.S. Pat. Nos. 4,444,887 and 4,716,111, each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. For example, it has been described that the homozygous deletion of the antibody heavy chain joining region (JH) gene in chimeric and germ-line mutant mice results in complete inhibition of endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Antibodies directed against the target of choice can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies, including, but not limited to, IgG1 (gamma 1) and IgG3. For an overview of this technology for producing human antibodies, see Lonberg and Huszar (Int. Rev. Immunol., 13:65-93 (1995)). In addition, companies such as Abgenix, Inc. (Freemont, Calif.) and Genpharm (San Jose, Calif.) can be engaged to provide human antibodies directed against a selected antigen using technology similar to that described above.

Alternatively, in some embodiments, the fragment of a non-human antibody for use in the disclosed CPRs can be humanized, where specific sequences or regions of the antibody are modified to increase similarity to an antibody naturally produced in a human. For instance, in the present disclosure, the scFv may comprise a non-human mammalian scFv, such as a mouse scFv. In some embodiments, the scFv is humanized.

### EXAMPLES

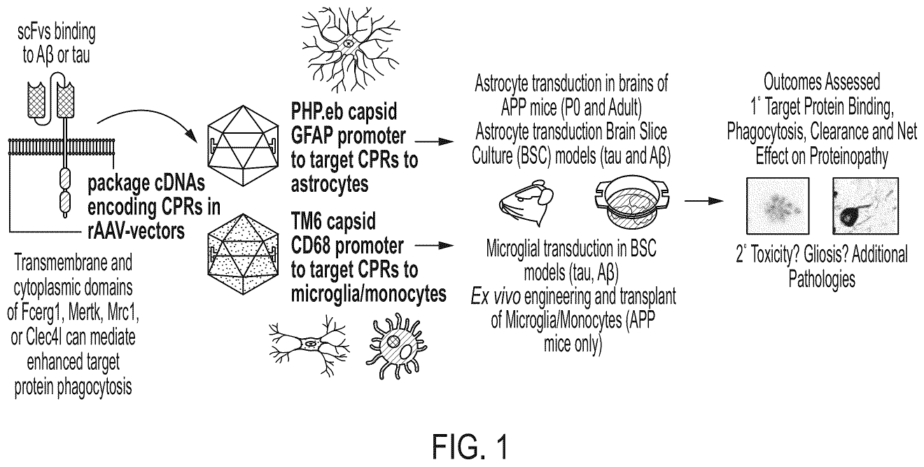

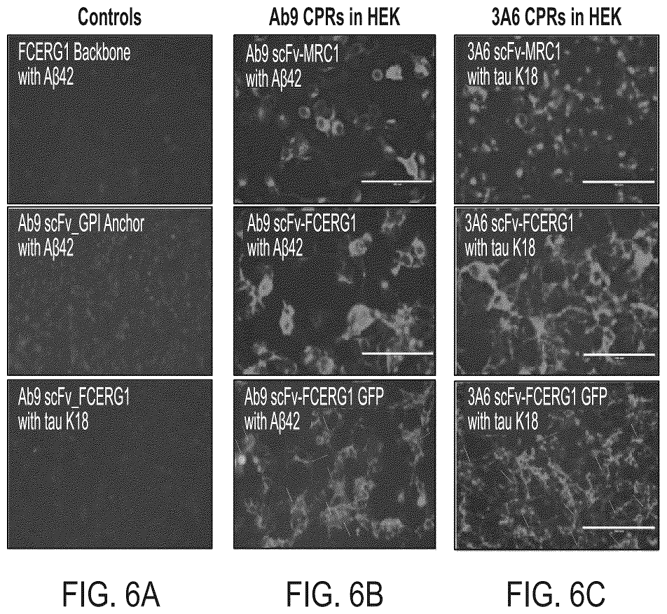

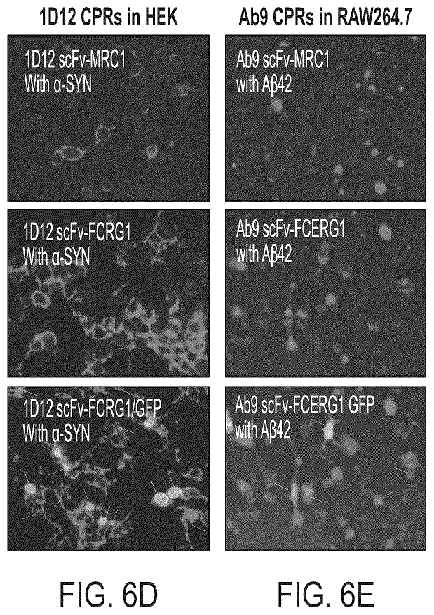

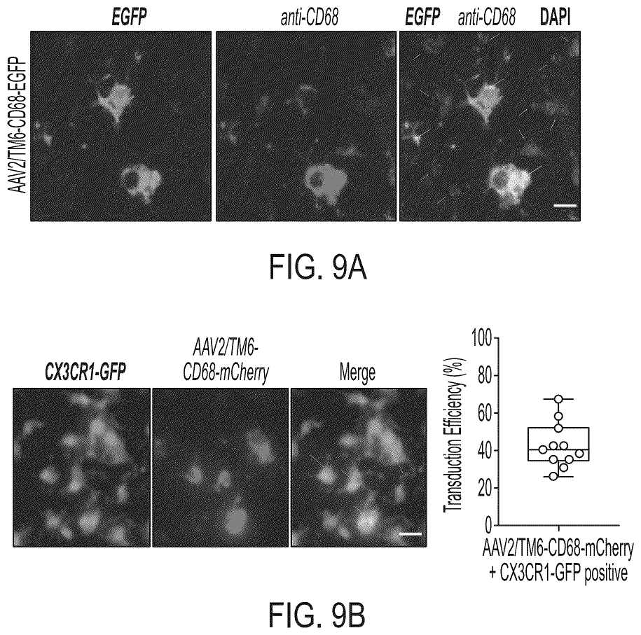

Enhancing clearance of the target protein via phagocytic mechanisms is arguably the most direct way to test the hypothesis that enhancing clearance of protein aggregates is therapeutically beneficial. This premise was directly evaluated using novel chimeric phagocytic receptors, or CPRs (see FIG. 1). Multiple CPRs targeting Aβ and tau were created and functionally validated. These CPRs are fusion molecules composed of an extracellular single chain variable fragment (scFv) that binds the target protein with high affinity that is fused via a linker to the transmembrane and cytoplasmic domains of different phagocytic receptors (FCER1G, MRC1, MERTK, CLEC4L, TREM2, M6PR, and DAP12, FIGS. 2-3). Notably, for each target protein, two or more CPRs have been identified (based on the different phagocytic receptor fusion) that selectively bind to the target protein and mediate rapid internalization of the targeted protein (FIGS. 6A-6E). In the central nervous system (CNS) both microglial cells and astrocytes can be phagocytic. Astrocytes are selectively transduced in neonatal and adult mice using appropriate rAAV vectors. Microglia cells remain difficult to transduce with high efficiency in vivo, but with novel rAAV vectors they can be transduced efficiently ex vivo (FIGS. 9A and 9B). These engineered microglial can then be studied ex vivo in relevant models or transplanted into the brains of mice. Similarly, peripheral monocytes can be manipulated ex vivo, and transplanted directly into the CNS.

Using select in vivo and ex vivo models of Aβ and tau pathology, including a novel brain slice culture models of tauopathy, the ability of CPRs is evaluated to have disease-modifying effects when delivered before pathology develops or when pathology is already robust.

These experiments leverage long-standing experience with i) rAAV vector development and delivery (13-15), ii) scFv and recombinant fusion protein engineering (16), and iii) in vivo and ex vivo models of Aβ and tau pathology (12, 15, 17). (See Goodwin M S, et al. Mol Neurodegener. 2020; 15(1):15, and Chakrabarty P, et al. PloS One. 2013; 8(6):e67680, both of which are incorporated herein by reference.) The main endpoint in these studies is to determine whether expression of CPRs may alter the pathologies.

AD is the most common form of dementia among the elderly. Over 5 million Americans currently have AD, and the number of cases is expected to rise to >13 million by 2050. The overall negative economic impact of AD is estimated to be more than $250 billion a year and is predicted to rise to more than $1 trillion if effective interventions are not identified (20). No effective AD therapies exist and many aspects of AD pathogenesis remain enigmatic. Pathological, genetic, and modeling studies support the hypothesis that AD is a complex proteinopathy. Indeed, both Aβ and tau, and in some cases other proteins (e.g., α-synuclein and TDP-43), aggregate and gradually accumulate in the AD brain (2, 21-23). This accumulation typically occurs in a characteristic temporal and spatial sequence with cortical amyloid deposition preceding development of cortical tau and neurodegeneration. The various proteins that accumulate are associated with complex cellular responses that ultimately lead to widespread neurodegeneration. However, there are still numerous gaps in the understanding of the mechanisms whereby the proteinopathy causes neurodegeneration. Of note, over 30 other neurodegenerative disorders including Parkinson's disease, many forms of amyotrophic lateral sclerosis, FTDP-17 MAPT, CBD, PSP, polyglutamine disorders, and many others are all thought to be neurodegenerative proteinopathies (2, 24).

Given the evidence that supports a cause and effect relationship between protein accumulation, aggregation and neurodegeneration in AD and many other neurodegenerative disorders, an obvious therapeutic approach is to try and enhance protein clearance mechanisms, especially those targeting the protein aggregate (6). In contrast to therapeutic strategies targeting protein production, strategies to enhance clearance may have a larger temporal window in which they could be effective as they might be able to work even in the setting of large amounts of preexisting pathology Herein, a novel approach is explored to enhance clearance via CPRs that efficiently target Aβ and tau (FIGS. 1, 2, and 6A-6E). The CPR approach is arguably the most direct way in which to assess whether enhanced phagocytosis can have beneficial effects in the setting of a neurodegenerative proteinopathy. As opposed to various passive or active immunotherapeutic approaches, the CPR approach directly links the targeting of a specific protein towards phagocytosis. Notably, it has been shown that there are limits to the efficacy of passive immunotherapies in terms of clearance of pre-existing deposits providing additional rationale for exploring CPRs as a more directed clearance strategy.

These studies rigorously test the hypothesis that enhancing phagocytic clearance of Aβ or tau within the brain, by either astrocytes or microglia/monocytes is potentially therapeutic. These experiments may demonstrate that CPRs can engage the target in vivo. Coupled with the ability to direct the expression to different cell types, these experiments provide a proof of concept for use of CPRs in treatment of neurodegenerative disorders. These studies represent the first disclosure of the functionality of CPRs expressed in vivo and CPRs for targets relevant to neurodegenerative proteinopathies.

Although astrocytes and microglial cells can be phagocytic, it has not been clear whether this phagocytic ability can be selectively harnessed for therapeutic benefit. CPRs have not been expressed, nor their function examined, in primary cells. In particular, CPRs have not been expressed in primary cells such as astrocytes or microglia.

Intracerebroventricular (ICV) injection of neonatal mice, and intravenous (IV) injection of adult mice, with novel rAAV vector reduces time and cost compared to stereotaxic surgery and results in effective transduction of astrocytes in the brains of these mice. Transduction of microglial cells in vivo has historically been challenging; however a modified rAAV capsid has recently been developed that transduces microglial cells ex vivo (12). Studies utilizing rAAV transduction of microglial cells (and monocytes) ex vivo and transplantation of these engineered cells back into the brain can establish a new paradigm for evaluating microglial function.

The experiments that follow take advantage of the brain slice culture models of intrinsic tau pathology and seeded “extrinsic” model of tauopathy to assess impact of expressed CPRs on tau. This model enables real time rapid and robust monitoring of binding and clearance events to validate mechanisms of action of the CPRs that would be challenging to accomplish in vivo.

### Engineering and Construction of Anti-Aβ and Anti-Tau CPRs.

Single chain recombinant antibody (scFv) is an antibody fragment lacking the constant region but retaining specificity and binding affinity of the full-size parent antibody. It contains the variable region of the heavy (VH) and the light chain (VL), connected by a flexible linker, and is expressed from a single gene. For the past several years, the laboratory has focused on evaluating the effects of anti-Aβ and anti-tau scFvs. It was demonstrated that scFvs are effectively expressed following neonatal rAAV delivery (26). In order to constitutively express a gene throughout the entire brain, the “SBT—somatic brain transgenesis” methodology was developed, by which, when injected into the cerebral ventricles of a newborn mouse, rAAV spreads throughout most neuronal populations of the brain and protein expression lasts for the lifetime of the animal without pathogenic effects. There is extensive data showing that these recombinant scFv's modify disease when delivered to the central nervous system (CNS) using rAAV therapies. It has been demonstrated, using this strategy, that scFvs targeting Aβ40, Aβ42, and most effectively, pan-Aβ, prevented the formation of amyloid plaques and reduced Aβ levels in two AD mouse models. It has also demonstrated that select anti-tau recombinant antibodies have the potential to reduce tau pathology and slow the progression of tau-induced neuronal dysfunction.

Multiple CPRs targeting Aβ and tau were designed and generated. Those scFv's were derived from mouse anti-pan-Aβ A139 (B11 scFv) and anti-4Rtau mouse 3A6 respectively. The transmembrane and cytoplasmic domains of different phagocytic receptors (FCER1G, MRC1, MERTK, CLEC4L) were fused to the C-terminues of the scFv (FIG. 2). It was validated that all constructs are functional by transient transfection of the plasmids into multiple cell lines (e.g., HEK293T, H4, CHO and monocyte/macrophage-like RAW264.7 cells) and evaluated the uptake of monomeric Aβ or tau, accordingly (FIG. 6A-6E).

Anti-AβCPRs and anti-tau CPRs only target their corresponding antigen, suggest the internalization is specific. Further, the internalization is the result of collaboration between the scFv domain and the phagocytic receptor domain. Receptor alone, scFv alone or membrane tethered scFv using glycosylphosphatidylinositol (GPI) anchor barely show any antigen internalization. Finally, the internalization is highly efficient. Even non-phagocytic cells (HEK, CHO and H4) transfected with CPRs internalize the antigen in a few minutes. Noticeable nonspecific Aβ and tau binding usually appear after 45 minutes and are diffusely distributed within those cells. Different phagocytic receptor domains may lead the antigen to different subcellular location or morphological changes as shown by comparing FCER1G and MRC1 CPRs transfected HEK cells.

### Example 1

### Experimental Design.

Explore whether astrocyte expressed CPRs targeting Aβ or tau enhance phagocyte clearance and alter the target pathology. In this aim it is tested whether the newly engineered CPRs enhance degradation of externally added fibrillary Aβ and tau when expressed in primary astrocytic culture, prevent phosphorylation of endogenous tau when expressed in astrocytes via AAV delivery to BSC as well as prevent and clear amyloid pathology when delivered to the mouse brain. Phagocytosis was thought to be limited to microglia in brain, however ongoing evidence suggests that astrocytes play a role in clearance of damaged neurons in injured brains (27, 28) or even under normal physiological conditions (29, 30). In AD astrocytes can phagocytose neurons containing Aβ (31), or endocytose and degrade monomeric and oligomeric Aβ through actin regulation (32, 33).

In the first step target engagement and phagocytic uptake was tested of all four CPR domains fused with anti-Aβ and anti-tau scFvs respectively. Astrocytes are prepared from neonatal (2-3 day old) mice as described (34). Briefly, primary cortical cultures are incubated at 37° C. in a humidified 5% (v/v) CO2 atmosphere until the cells reached confluence. The medium is changed every 3d with rigorous shaking to remove contaminating non-adherent cells from adherent astrocytes. The purity of astrocyte enriched cultures are tested by glial fibrillary acidic protein (GFAP) immunoreactivity. Primary astrocytes plated onto cover slips are then transduced with CPR rAAV-PHP.eB under the control of a GFAP promoter. Transduction efficiency is examined by Western blot of CPRs or GFP overexpression, which is from co-transduced EGFP virus or CPR-EGFP fusion construct virus. GPI anchored scFv or receptor domain virus serve as control. 72 h after transduction, ALEXA FLUOR-555 labelled Aβ42 oligomers, Aβ42 aggregates, or tau K18 fibrils are added onto astrocytes. Cellular uptake of Aβ and tau are evaluated with flow cytometry-based assay and confocal microscope. For the image analysis, multiple fields per slip are randomly chosen and only cells with clear intracellular antigen are considered internalization positive cells.

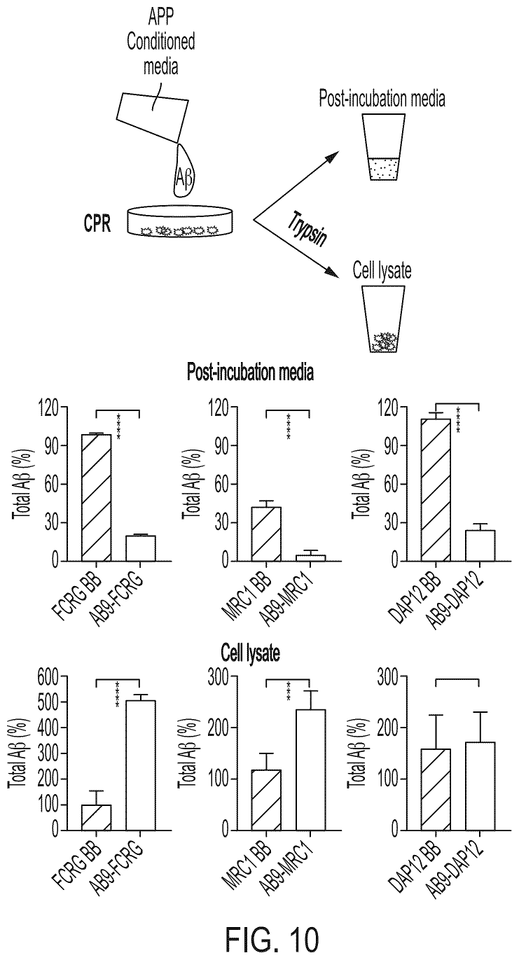

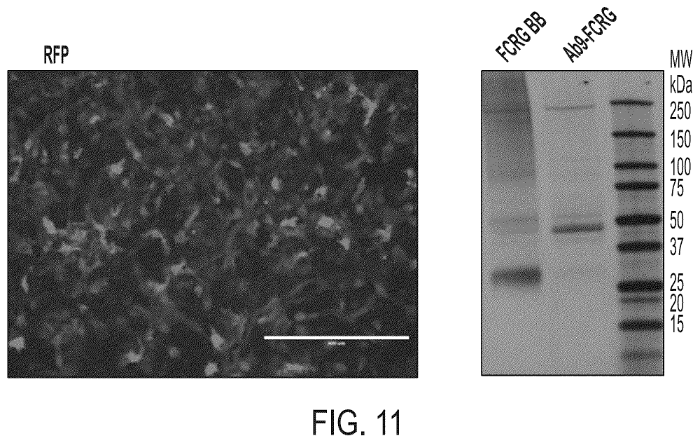

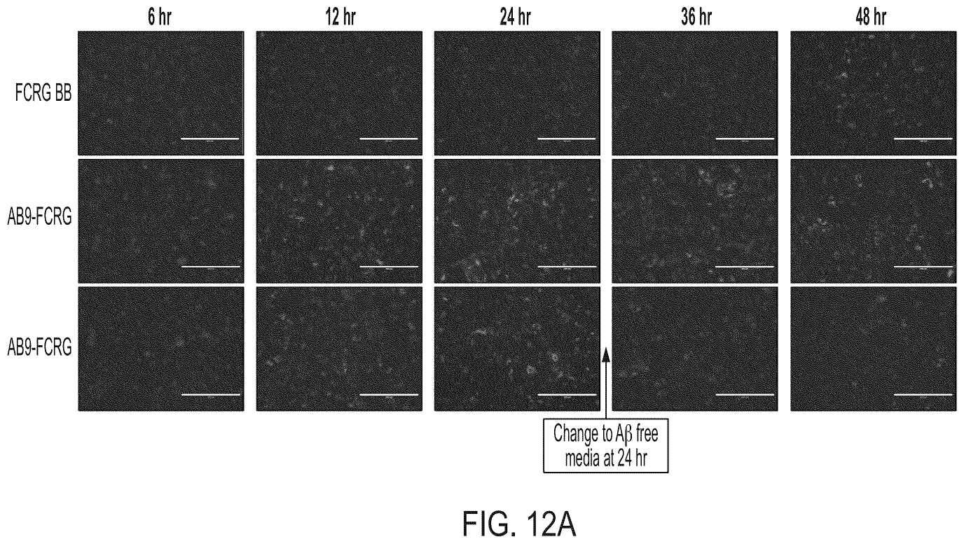

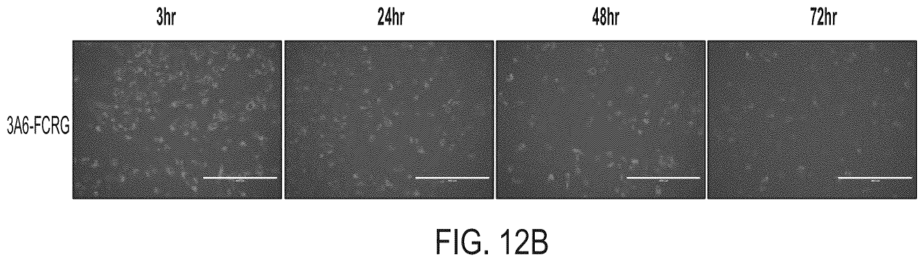

The degree of cellular endocytosis mediated by each CPR was quantified in an in vitro assay. As shown in FIG. 10, amyloid precursor protein (APP)-condition media was added to HEK cells transfected with Aβ9scFv-CPRs or control plasmid Aβ9scFv-CD8, which is not phagocytic, and incubated overnight. The conditioned media contained Dulbecco's Modified Eagle Medium (DMEM) with 10% FBS and 1% pen-strep, along with secreted amyloid beta (Aβ). Cell media was spun to separate lysate and post-incubation media. Then, the total Aβ in post-incubation media (top) and cell lysate (bottom) were measured by ELISA. Aβ levels in cells treated with control plasmid Aβ9scFv-CD8 (which is not phagocytic) were set as 100%, and the percentages of A139 concentration following CPR treatment are indicated as against the control levels on the y-axis. DAP12, MRC1, and FCERG backbones (“BB”), which are not phagocytic, also served as controls.

The ELISA results showed a pattern in which Aβ levels typically decreased in the media and increased in the cell lysate following treatment with Aβ9scFv-CPRs (FIG. 10). This pattern was particularly evident for FCERG and MRC1 CPRs. Yet each CPR exhibited different abilities to uptake Aβ. The increase of intracellular Aβ was not in all cases accompanied by a decreasing media Aβ concentration for the DAP12 CPR, suggesting each CPR might lead Aβ to different intracellular degradation pathway.

Since different subcellular distribution of the antigen by different CPRs was noticed, antigen subcellular location is further investigated using cell and organelle markers.

Astrocytes transduction in vivo via rAAV-PHP.eB-GFAP. Widespread expression in the adult brain can be achieved following IV injection of PHP.eB-CBA-GFP virus (see BSC images in FIGS. 9A and 9B). Expression was observed in all cell types, including astrocytes.

If astrocytes expressing engineered CPRs exhibit phagocytic functions, it is examined if and how antigen uptake affects cultured astrocyte physiological and pathological activity including: 1) The fate of internalized antigen i.e. Aβ degradation and tau phosphorylation; 2) proinflammatory factor level such as IL-1(3, IL-6, TNF-α, and TGF-β; 3) level of protease, receptor and other protein involved in Aβ and tau metabolism, such as MMP-2, MMP-9, LRP-1, and ApoE.

Organotypic slice culture (BSC) as a model of tau aggregation and pathology. An approach was developed to recapitulate neurofibrillary tangles (NFTs) like those seen in human tauopathies in a three-dimensional, cytoarchitecturally, and functionally intact system. Organotypic brain slice cultures (BSCs) were transduced using rAAVs to express human wild-type (WT) or mutant pro-aggregant tau and maintained them in vitro for several weeks. BSCs may be prepared according to the procedures of Croft C L, et al. J. Exp. Med. 2019; 216(3):539-55, incorporated herein by reference.

BSCs transduced with P301L/S320E-tau and P301L/S320F/A153T-tau mutants progressively develop an abundance of hyperphosphorylated, sarkosyl-insoluble, filamentous (by EM), Thio-S positive NFT inclusions between 14 and 28 days in vitro. BSCs transduced with P301L tau and seeded with tau filaments develop similar NFT inclusions.

For in vivo evaluation of CPRs, the organotypic slice culture model of tau pathology and fast and aggressive amyloid depositing transgenic mouse model CRND8 are used. Brain slices were prepared as previously described from postnatal day 8-9 non-transgenic (ntg) B6C3F1 mice (12). Slices are plated randomly to contain three slices per semi-porous membrane insert in 6-well sterile culture plates. rAAV-PHP.eB-GFAP-anti-tau CPRs (FCER1G, MRC1, MERTK, and CLEC4L with a non-specific CPR, anti-tau scFv alone, and GFP as controls) are applied to BSCs on the first day of culture (0 days in vitro, or DIV) by adding into the culture medium (1 to 2×10¹⁰ viral genomes per well containing three BSCs). Exemplary results are shown in FIGS. 6A-6E. Use of a GFAP promoter to control expression of the transgene ensures that production of the CPR protein is specific to astrocytes (see FIG. 8).

Tau pathology is induced by transducing the slices with rAAV2/8 vectors encoding the P301L/S320E-tau mutant prior to or simultaneously with rAAV-CPR and examine the timeline of efficient clearance of tau accumulation and aggregation. The question asked is at what point along the development of mature tau tangle pathology do CPRs clear the tau aggregates. By transducing slices with fluorescently tagged tau, tau pathology is live-imaged and clearance is monitored by CPR. In parallel, pathology is assessed at 3 time points, 7, 14, and 21 days after the initiation of pathology by immunohistochemical and biochemical methods. At each time point slices are imaged to assess tau tangle pathology, neuronal death, and astrocyte activation using glial fibrillary acid protein (GFAP). In addition, slices are harvested at each time point to biochemically analyze levels of soluble, insoluble, and 69hosphor-tau by Western blot as well as markers of astrocyte activation, inflammation, and cell death.

Additionally, to assess the effect of CPRs on tau clearance in the seeded model of tauopathy, CPRs are expressed in BSCs and fluorescently tagged tau aggregates are added. Tau clearance is live-imaged and rate of phagocytosis is quantified. In parallel, levels of tau pathology are biochemically analyzed by Western blot, markers of astrocyte activation, and cell death. A large number of biological replicates are included to ensure reporting of significant differences.

In vivo CPR prevention and clearance assessment. To evaluate the ability of CPR to prevent amyloid pathology, astrocytes are selectively transduced using rAAV-PHP.eB-GFAP-CPR and evaluate effects on amyloid pathology in CRND8 mice, a transgenic model that overexpresses mutant human amyloid protein precursor (APP) at levels approximately 5-fold higher than endogenous murine APP (see Chakrabarty, et al. Neuron 85, 519-533 (2015), incorporated herein by reference). For the prevention paradigm newborn CRND8 are injected with rAAV-PHP.eB-GFAP-CPR (with FCER1G, MRC1, MERTK, and CLEC4L with a non-specific CPR, anti-pan Aβ scFv alone, and GFP used as controls) (ICV, 4×10¹⁰ viral genomes/pup) and assess Aβ levels and plaque burden at 3 months. Each construct is delivered to 4-5 litters, resulting in −20 transgenic mice, of whom half will reach 3 months of age. Based on extensive experience with this strain, this cohort size allows to detect significant changes of 25% in amyloid levels. Small sub-cohorts on non-transgenic littermates at 4 weeks old are harvested to evaluate transduction and tropism.

At 3 months, the age at which substantial plaque formation is typically observed in these mice, one hemibrain is fixed and stained for amyloid immunoreactivity burden with anti-pan-Aβ antibody, Aβ5-biotin, and Thio-S. The other hemibrain is fractionated and Aβ levels are quantified by ELISA from RIPA-soluble fraction, SDS-soluble, and SDS-insoluble, formic acid-soluble fractions. Astrogliosis and microgliosis are evaluated using glial fibrillary acid protein (GFAP) and ionized calcium-binding molecule-1 (IBA-1) antibodies, respectively. Image analysis is done following scanning whole slides with the Aperio System and analysis of immunoreactivity burden by ImageScope program (Aperio, CA). Neuron counts in specific areas of the hippocampus and cortex where neurodegenerative may be observed by gross H&E analysis are also assessed quantitatively using NeuN antibody and determined by double-blinded quantification. Detailed calculations of mouse numbers are presented in the Vertebrate animals section. Control groups were injected with a non-specific CPR, anti-pan Aβ alone, and GFP. It is expected that each CPR has a differential effect on Aβ.

Based on these results the most effective anti-AβCPR agent can be pinpointed. To test whether CPRs are functional in a therapeutic paradigm, one or two of the most promising CPR from prevention study, and the appropriate controls, are administered intraocularly or into the tail vein into 4 month old CRND8 mice. At 4 months of age CRND8 mice have moderate levels of deposits, by 6 months they have significant amyloid accumulation. Amyloid burden and Aβ levels are assessed at 2 months post injection and compared to the non-specific CPR control group. These controls are designed to test whether non-specific activation of astrocytes is beneficial in targeting amyloid pathology. Additionally, control groups are injected with scFv's alone and GFP.

Given the above-described positive preliminary results and the demonstrated success in performing all the methods, experiments to assess the effect on prevention of aggregation and clearance of existing pathology are ongoing. CPRs activate astrocytes and microglia and it is possible that the non-specific CPR may have an effect on amyloid pathology. Nevertheless, the role of phagocytosis in amyloid clearance and the specific CPR domain may become better understood. Experiments are ongoing to optimize the CPRs of the disclosure to induce minimal off-target effects.

### Example 2

CPRs May Bind AD or Tau when Expressed in Microglia or Monocytes and Alter the Proteinopathy in Ex Vivo Brain Slice Culture Models or In Vivo Following Transplantation of the CPR Expressing Microglia/Monocytes into the Brain of the Appropriate Proteinopathy Mouse Model.

This experiment evaluated whether the CPRs enhance microglial phagocytosis of amyloid plaques and tau aggregates. Multiple genetic association studies have identified numerous loci that harbor genes that encode proteins of known immune function that alter the risk of developing AD and other neurodegenerative proteinopathies (35). Microglial cells are the primary immune cells of the central nervous system and function as macrophages. They respond to foreign material or cellular debris by changing their morphology, becoming activated, migrating to the site of damage, clearing the foreign material and cellular debris through phagocytosis, and secreting signals to increase response to the insult (36). Activated microglia have been described as a common feature of many neurodegenerative diseases (37-39). To enhance microglial activity, microglia are transduced with CPRs (i.e., by AAV vectors encoding the CPRs). Transducing microglia in vivo remains a technical challenge, but a novel mutant capsid has been developed that transduces microglial cells in culture and ex vivo brain slices (FIGS. 9A and 9B). This allows testing of the approach of CPRs in a BSC model of model of tauopathy and to engineer microglial cells ex vivo and transplant into AD mouse model to increase phagocytic clearance of amyloid.

Initially it is established that microglia and monocytes are efficiently transduced with CPRs. It has been shown that rAAV2/TM6 (rAAV2/6 with three mutations, Y731F/Y705F/T492V) with CD68 promoter efficiently transduces microglial cells. Microglial culture is prepared from P2 non transgenic mouse brains as previously described. Peripheral blood or bone marrow derived monocytes are prepared as described (40, 41). The outline of Example 1 is followed to determine the function of CPRs on microglia or monocytes cell culture.

In the next stage microglia activating CPRs are evaluated in the organotypic brain slice culture. Brain slices are prepared from postnatal day 8-9 Cx3Cr1-EGFP mice. Monocytes and microglial cells from these mice express GFP, which allows us to image Aβ phagocytosis and examine their phenotypes. Slices are plated randomly to contain three slices per semi-porous membrane insert in six-well sterile culture plates. To selectively express CPR in microglia, slices are transduced with rAAV2/1 TM6-CD68-CPR. Virus particles are applied to BSCs on the first day of culture (0 DIV) by adding into the culture medium 1-2×10¹⁰ viral genomes per well containing three BSCs. It has been previously shown that microglial transduction by rAAV2/1-TM6-EGFP resulted in 40% transduction efficiency (FIGS. 9A-9B).

A second way to introduce CPRs to BSCs is replenishment of BSCs with primary mouse microglia and monocytes. Endogenous microglial cells on BSCs are depleted pharmacologically with clodronate, which results in 70% reduction of microglial cells. Primary microglial cells transduced with CPRs is then be introduced and assayed.

Similar to Example 1, the CPR is co-transduced with rAAV-encoded mutant, pro-aggregant tau. The effect of CPR on tau clearance is then analyzed biochemically and immunohistochemically.

Examine feasibility of CPR approach of enhancing phagocytosis through ex-vivo engineering of microglia/monocytes and transplanting them into adult CRND8 mice. Similar to Example 1, one or two best candidate anti-AβCPRs are tested. 1×10⁶ CPR-engineered microglia cells or monocytes from Cx3Cr1-EGFP mice, prepared according to C2b1 are injected into lateral ventricles of 4-month-old CRND8 mice, as described in Mancuso R, et al. Nat Neurosci. 2019; 22(12): 2111-16, which is incorporated herein by reference. Nonspecific CPR-expressing microglia as well naïve microglia serve as controls. Transplantation efficiency is assessed in a small sub-cohort by Western blot or immunohistochemistry with anti-GFP and anti-FLAG antibodies. Effects on pathology two months after injection were assessed as described below.

rAAV particles encoding CPRs containing FCERG-scFv Aβ9 and MRC1-scFv Aβ9 under the control of a GFAP promoter were administered by stereotaxic surgery to 8-month-old CRND8 mice. 2 months later, the brains of these mice were harvested for biochemical and immunohistochemical analysis. One hemibrain tissue was extracted and fixed, and paraffin-embedded tissue was stained with antibodies. Consecutive sections were stained with the following markers: anti-FLAG to detect expression, pan-Abeta antibody to detect amyloid aggregation, GFAP, and IBA-1. The amyloid immunoreactivity burden was quantified using ImageScope analysis in three non-consecutive sections per mouse. Untreated mice (non-inj) were used as a control.

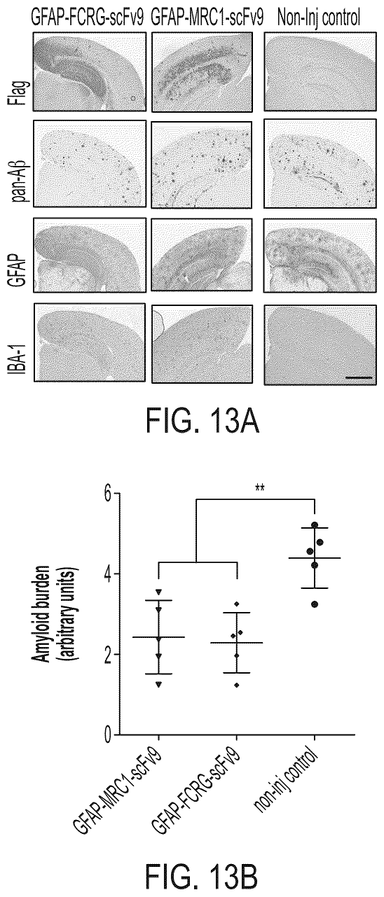

The results are shown in FIGS. 13A and 13B indicate that CPRs containing FCERG-scFv Aβ9 and MRC1-scFv Aβ9 enhanced the phagocytosis and clearance of amyloid from mouse models in vivo.

### Alternative Strategies.

Some researchers have suggested that aged microglia can be replaced or reinforced by exogenously introduced cells capable of carrying out the functions of young, non-senescent microglia (43, 44). Studies in BSCs have shown that brain slices deprived of microglia can be again replenished by exogenously introduced cultured microglia which are able to colonize the brain parenchyma, acquire a ramified morphology, and confer neuroprotection to excitotoxic injury (45, 25). Mice of endogenous microglia are also depleted prior to transplantation to achieve better engraftment. Additionally, microglial transplant expressing the (control) non-specific CPR might have an effect on clearance of amyloid, but it is expected that these microglia may become “fatigued” quickly, whereas specific CPR will facilitate prolonged activity of transplanted microglia.

### REFERENCES

1. Golde T E, Borchelt D R, Giasson B I, Lewis J. Thinking laterally about neurodegenerative proteinopathies. J Clin Invest. 2013; 123(5):1847-55.
2. Chiti F, Dobson C M. Protein misfolding, functional amyloid, and human disease. Annu Rev Biochem. 2006; 75:333-66.
3. Forman M S, Trojanowski J Q, Lee V M. Neurodegenerative diseases: a decade of discoveries paves the way for therapeutic breakthroughs. Nat Med. 2004; 10(10):1055-63.
4. Ross C A, Pickart C M. The ubiquitin-proteasome pathway in Parkinson's disease and other neurodegenerative diseases. Trends Cell Biol. 2004; 14(12):703-11.
5. Golde T E, DeKosky S T, Galasko D. Alzheimer's disease: The right drug, the right time. Science. 2018; 362(6420):1250-1.
6. Eisele Y S, Monteiro C, Fearns C, Encalada S E, Wiseman R L, Powers E T, et al. Targeting protein aggregation for the treatment of degenerative diseases. Nat Rev Drug Discov. 2015; 14(11):759-80.
7. Golde T E. Overcoming translational barriers impeding development of Alzheimer's disease modifying therapies. J Neurochem. 2016; 139 Suppl 2:224-36.
8. Cummings J L, Morstorf T, Zhong K. Alzheimer's disease drug-development pipeline: few candidates, frequent failures. Alzheimers Res Ther. 2014; 6(4):37.
9. Doody R S, Thomas R G, Farlow M, Iwatsubo T, Vellas B, Joffe S, et al. Phase 3 trials of solanezumab for mild-to-moderate Alzheimer's disease. N Engl J Med. 2014; 370(4):311-21.
10. Egan M F, Kost J, Tariot P N, Aisen P S, Cummings J L, Vellas B, et al. Randomized Trial of Verubecestat for Mild-to-Moderate Alzheimer's Disease. N Engl J Med. 2018; 378(18):1691-703.
11. Sevigny J, Chiao P, Bussiere T, Weinreb P H, Williams L, Maier M, et al. The antibody aducanumab reduces Abeta plaques in Alzheimer's disease. Nature. 2016; 537(7618):50-6.
12. Croft C L, Cruz P E, Ryu D H, Ceballos-Diaz C, Strang K H, Woody B M, et al. rAAV-based brain slice culture models of Alzheimer's and Parkinson's disease inclusion pathologies. J Exp Med. 2019; 216(3):539-55.
13. Goodwin M S, Croft C L, Futch H S, Ryu D, Ceballos-Diaz C, Liu X, et al. Utilizing minimally purified secreted rAAV for rapid and cost-effective manipulation of gene expression in the CNS. Mol Neurodegener. 2020; 15(1):15.
14. Ayers J I, Fromholt S, Sinyayskaya 0, Siemienski Z, Rosario A M, Li A, et al. Widespread and efficient transduction of spinal cord and brain following neonatal AAV injection and potential disease modifying effect in ALS mice. Mol Ther. 2015; 23(1):53-62.
15. Chakrabarty P, Rosario A, Cruz P, Siemienski Z, Ceballos-Diaz C, Crosby K, et al. Capsid serotype and timing of injection determines AAV transduction in the neonatal mice brain. PloS One. 2013; 8(6):e67680.
16. Levites Y, Jansen K, Smithson L A, Dakin R, Holloway V M, Das P, et al. Intracranial adeno-associated virus-mediated delivery of anti-pan amyloid beta, amyloid beta40, and amyloid beta42 single-chain variable fragments attenuates plaque pathology in amyloid precursor protein mice. J Neurosci. 2006; 26(46):11923-8.
17. Dawson T M, Golde T E, Lagier-Tourenne C. Animal models of neurodegenerative diseases. Nat Neurosci. 2018; 21(10):1370-9.
18. June C H, Sadelain M. Chimeric Antigen Receptor Therapy. N Engl J Med. 2018; 379(1):64-73.
19. Morrissey M A, Williamson Aβ, Steinbach A M, Roberts E W, Kern N, Headley M B, et al. Chimeric antigen receptors that trigger phagocytosis. elife. 2018; 7:e36688.
20. Alzheimer's A. 2016 Alzheimer's disease facts and figures. Alzheimers Dement. 2016; 12(4):459-509.
21. Singleton A B, Farrer M, Johnson J, Singleton A, Hague S, Kachergus J, et al. alpha-Synuclein locus triplication causes Parkinson's disease. Science. 2003; 302(5646):841.
22. Baker M, Mackenzie I R, Pickering-Brown S M, Gass J, Rademakers R, Lindholm C, et al. Mutations in progranulin cause tau-negative frontotemporal dementia linked to chromosome 17. Nature. 2006; 442(7105):916-9.
23. Rovelet-Lecrux A, Hannequin D, Raux G, Le Meur N, Laquerriere A, Vital A, et al. APP locus duplication causes autosomal dominant early-onset Alzheimer disease with cerebral amyloid angiopathy. Nat Genet. 2006; 38(1):24-6.
24. Ross C A, Poirier M A. Protein aggregation and neurodegenerative disease. Nat Med. 2004; 10 Suppl:S10-7.
25. Vinet J, Weering H R, Heinrich A, Kalin R E, Wegner A, Brouwer N, et al. Neuroprotective function for ramified microglia in hippocampal excitotoxicity. J Neuroinflammation. 2012; 9:27.
26. Levites Y, Das P, Price R W, Rochette M J, Kostura L A, McGowan E M, et al. Anti-Abeta42- and anti-Abeta40-specific mAbs attenuate amyloid deposition in an Alzheimer disease mouse model. J Chin Invest. 2006; 116(1):193-201.
27. Cheng H W, Jiang T, Brown S A, Pasinetti G M, Finch C E, McNeill T H. Response of striatal astrocytes to neuronal deafferentation: an immunocytochemical and ultrastructural study. Neuroscience. 1994; 62(2):425-39.
28. Morizawa Y M, Hirayama Y, Ohno N, Shibata S, Shigetomi E, Sui Y, et al. Reactive astrocytes function as phagocytes after brain ischemia via ABCAl-mediated pathway. Nat Commun. 2017; 8(1):28.
29. Al-Ali S Y, Al-Zuhair A G, Dawod B. Ultrastructural study of phagocytic activities of young astrocytes in injured neonatal rat brain following intracerebral injection of colloidal carbon. Glia. 1988; 1(3):211-8.
30. Nguyen J V, Soto I, Kim K Y, Bushong E A, Oglesby E, Valiente-Soriano F J, et al. Myelination transition zone astrocytes are constitutively phagocytic and have synuclein dependent reactivity in glaucoma. Proc Natl Acad Sci USA. 2011; 108(3):1176-81.
31. Nagele R G, D'Andrea M R, Lee H, Venkataraman V, Wang H Y. Astrocytes accumulate A beta 42 and give rise to astrocytic amyloid plaques in Alzheimer disease brains. Brain Res. 2003; 971(2):197-209.
32. Lee S J, Seo B R, Koh J Y. Metallothionein-3 modulates the amyloid beta endocytosis of astrocytes through its effects on actin polymerization. Mol Brain. 2015; 8(1):84.
33. Wyss-Coray T, Loike J D, Brionne T C, Lu E, Anankov R, Yan F, et al. Adult mouse astrocytes degrade amyloid-beta in vitro and in situ. Nat Med. 2003; 9(4):453-7.
34. Schildge S, Bohrer C, Beck K, Schachtrup C. Isolation and culture of mouse cortical astrocytes. J Vis Exp. 2013(71).
35. Golde T E. Harnessing Immunoproteostasis to Treat Neurodegenerative Disorders. Neuron. 2019; 101(6):1003-15.
36. Perea J R, Llorens-Martin M, Avila J, Bolos M. The Role of Microglia in the Spread of Tau: Relevance for Tauopathies. Front Cell Neurosci. 2018; 12:172.
37. Glass C K, Saijo K, Winner B, Marchetto M C, Gage F H. Mechanisms underlying inflammation in neurodegeneration. Cell. 2010; 140(6):918-34.
38. Maragakis N J, Rothstein J D. Mechanisms of Disease: astrocytes in neurodegenerative disease. Nat Clin Pract Neurol. 2006; 2(12):679-89.
39. Ransohoff R M, Perry V H. Microglial physiology: unique stimuli, specialized responses. Annu Rev Immunol. 2009; 27:119-45.
40. Houthuys E, Movahedi K, De Baetselier P, Van Ginderachter J A, Brouckaert P. A method for the isolation and purification of mouse peripheral blood monocytes. J Immunol Methods. 2010; 359(1-2):1-10.
41. Wagner M, Koester H, Deffge C, Weinert S, Lauf J, Francke A, et al. Isolation and intravenous injection of murine bone marrow derived monocytes. J Vis Exp. 2014(94).
42. Mancuso R, Van Den Daele J, Fattorelli N, Wolfs L, Balusu S, Burton 0, et al. Stem-cell-derived human microglia transplanted in mouse brain to study human disease. Nat Neurosci. 2019; 22(12):2111-6.
43. Takata K, Kitamura Y, Yanagisawa D, Morikawa S, Morita M, Inubushi T, et al. Microglial transplantation increases amyloid-beta clearance in Alzheimer model rats. FEBS Lett. 2007; 581(3):475-8.
44. Wong W T. Microglial aging in the healthy CNS: phenotypes, drivers, and rejuvenation. Front Cell Neurosci. 2013; 7:22.
45. Masuch A, van der Pijl R, Funer L, Wolf Y, Eggen B, Boddeke E, et al. Microglia replenished OHSC: A culture system to study in vivo like adult microglia. Glia. 2016; 64(8):1285-97.

### EMBODIMENTS AND EQUIVALENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms. All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A chimeric receptor comprising:
(i) an intracellular phagocytic signaling domain,
(ii) an extracellular binding domain comprising a single-chain variable antibody fragment (scFv) having affinity for an amyloid beta peptide, tau protein or α-synuclein protein, and
(iii) a transmembrane domain positioned between and covalently linked to the intracellular phagocytic signaling domain and the extracellular binding domain,
wherein the scFv comprises an amino acid sequence having at least 90%, at least 92.5%, at least 95%, at least 98%, or at least 99% sequence identity to any of SEQ ID NOs: 1-6.

2. A chimeric receptor comprising:
(i) an intracellular phagocytic signaling domain,
(ii) an extracellular binding domain comprising a single-chain variable antibody fragment (scFv) having affinity for an amyloid beta peptide, tau protein or α-synuclein protein, and
(iii) a transmembrane domain positioned between and covalently linked to the intracellular phagocytic signaling domain and the extracellular binding domain,
wherein the intracellular phagocytic signaling domain is derived from the TREM2 (triggering receptor expressed on myeloid cells-2) receptor.

3. A chimeric receptor comprising:
(i) a first intracellular phagocytic signaling domain,
(ii) an extracellular binding domain comprising a single-chain variable antibody fragment (scFv) having affinity for an amyloid beta peptide, tau protein or α-synuclein protein,
(iii) a transmembrane domain positioned between and covalently linked to the intracellular phagocytic signaling domain and the extracellular binding domain, and
(iv) a second intracellular phagocytic signaling domain fused to the first intracellular phagocytic signaling domain.

4. The chimeric receptor of claim 2 or 3, wherein the scFv comprises an amino acid sequence having at least 90%, at least 92.5%, at least 95%, at least 98%, or at least 99% sequence identity to any of SEQ ID NOs: 1-6.

5. The chimeric receptor of any one of claims 1-4, wherein the transmembrane domain comprises a hinge domain.

6. The chimeric receptor of any one of claims 1-4, wherein the transmembrane domain is covalently linked to the intracellular phagocytic signaling domain and the extracellular binding domain by one or more linkers.

7. The chimeric receptor of claim 6, wherein the one or more linkers comprises the amino acid sequence GGGS (SEQ ID NO: 55) or GGGSGGGS (SEQ ID NO: 56).

8. The chimeric receptor of any one of claims 1-7, wherein the scFv has affinity for an amyloid beta peptide (Aβ).

9. The chimeric receptor of any one of claims 1-8, wherein the scFv has affinity for an amyloid beta peptide 9 (Aβ9).

10. The chimeric receptor of any one of claims 1-7, wherein the scFv has affinity for tau protein.

11. The chimeric receptor of any one of claims 1-7, wherein the scFv has affinity for α-synuclein protein.

12. The chimeric receptor of any one of claims 1-11, wherein the scFv comprises the amino acid sequence of any of SEQ ID NOs: 1-6.

13. The chimeric receptor of any one of claims 1 and 3-12, wherein the first intracellular phagocytic signaling domain comprises a TREM2 signaling domain, a MRC1 signaling domain, a MEGF10 signaling domain, a DAP12 signaling domain, a MERTK signaling domain, a FCεRIγ (or FCERG) signaling domain, a M6PR signaling domain, or a CLEC4L signaling domain.

14. The chimeric receptor of any one of claims 1 and 3-13, wherein the first intracellular phagocytic signaling domain comprises a TREM2 signaling domain.

15. The chimeric receptor of claim 3, wherein the first intracellular phagocytic signaling domain comprises a TREM2 signaling domain, a MRC1 signaling domain, a MEGF10 signaling domain, a DAP12 signaling domain, a MERTK signaling domain, a FCERG signaling domain, a M6PR signaling domain, or a CLEC4L signaling domain;
and the second intracellular phagocytic signaling domain comprises a TREM2 signaling domain, a MRC1 signaling domain, a MEGF10 signaling domain, a DAP12 signaling domain, a MERTK signaling domain, a FCERG signaling domain, a M6PR signaling domain, or a CLEC4L signaling domain.

16. The chimeric receptor of claim 3 or 15, wherein the first intracellular phagocytic signaling domain comprises a FCERG signaling domain, and the second intracellular phagocytic signaling domain comprises a MRC1 signaling domain.

17. The chimeric receptor of any one of claims 1-16, wherein the chimeric receptor comprises an amino acid sequence having at least 85%, at least 90%, at least 92.5%, at least 95%, at least 98%, or at least 99% sequence identity to any of SEQ ID NOs: 26-43.

18. The chimeric receptor of any one of claims 1-16, wherein the chimeric receptor comprises a second extracellular binding domain.

19. The chimeric receptor of any one of claims 1 and 3-18, wherein the first intracellular phagocytic signaling domain comprises a FCERG signaling domain.

20. The chimeric receptor of any one of claims 1 and 3-18, wherein the first intracellular phagocytic signaling domain comprises a CLEC4L signaling domain.

21. The chimeric receptor of any one of claims 1-20, wherein the transmembrane domain is a TREM2 transmembrane domain, a MRC1 transmembrane domain, a MEGF10 transmembrane domain, a DAP12 transmembrane domain, a MERTK transmembrane domain, a CD8 transmembrane domain, a M6PR transmembrane domain, or a CLEC4L transmembrane domain.

22. A polynucleotide encoding the chimeric receptor of any one of claims 1-21.

23. The polynucleotide claim 22 further comprising a sequence encoding a secretion signal peptide.

24. The polynucleotide of claim 22 or 23 further comprising one or more tags.

25. The polynucleotide of claim 24, wherein the one or more tags comprise a FLAG tag.

26. The polynucleotide of any one of claims 22-25 further comprising one or more furin cleavage sites.

27. The polynucleotide of any one of claims 22-26, wherein the signal peptide is selected from a TREM2 signal peptide, a MRC1 signal peptide, a MEGF10 signal peptide, a DAP12 signal peptide, a MERTK signal peptide, a CD8 signal peptide, a M6PR signal peptide, and CD209 (DC-SIGN).

28. The polynucleotide of any one of claims 22-27, wherein the signal peptide is a TREM2 signal peptide.

29. The polynucleotide of any one of claims 22-28, wherein the polynucleotide comprises a nucleic acid sequence having at least 85%, at least 90%, at least 92.5%, at least 95%, at least 98%, or at least 99% sequence identity to any of SEQ ID NOs: 45-54.

30. An expression vector comprising the polynucleotide of any one of claims 22-29.

31. The vector of claim 30, wherein the vector comprises a heterologous promoter driving expression of the polynucleotide.

32. The vector of claim 30 or 31, wherein the promoter is selected from CD68 promoter, Glial Fibrillary Acidic protein (GFAP) promoter, CMV enhancer/chicken Beta-actin (CAG) promoter, Microtubule-associated protein 2 (MAP2) promoter or synapsin 1 (SYN) promoter.

33. The vector of any one of claims 30-32, wherein the heterologous promoter comprises a Glial Fibrillary Acidic protein (GFAP) promoter.

34. The vector of any one of claims 30-33, wherein the heterologous promoter is active in astrocyte cells.

35. The vector of any one of claims 30-32, wherein the heterologous promoter comprises a CD68 promoter.

36. The vector of any one of claims 30-32 and 35, wherein the heterologous promoter is active in microglia cells.

37. A recombinant adeno-associated viral (rAAV) particle comprising the polynucleotide of any one of claims 22-29 or the vector of any one of claims 30-36.

38. The rAAV particle of claim 37 further comprising an PHP.eB capsid.

39. The rAAV particle of claim 37 further comprising an AAV6(Y705F+Y731F+T492V) (TM6) capsid.

40. The rAAV particle of claim 37 further comprising a TM2 capsid.

41. A modified cell comprising a chimeric receptor comprising:
(i) an intracellular phagocytic signaling domain,
(ii) an extracellular binding domain comprising a single-chain variable antibody fragment (scFv) having affinity for an amyloid beta peptide, a tau protein or α-synuclein protein, and
(iii) a transmembrane domain positioned between and covalently linked to the intracellular phagocytic signaling domain and the extracellular binding domain;
wherein the modified cell is an astrocyte.

42. A modified cell comprising the chimeric protein of any one of claims 1-21, the polynucleotide of any one of claims 22-29, the vector of any one of claims 30-36, or the rAAV particle of any one of claims 37-39, 72, and 73.

43. A modified cell expressing on the surface of the modified cell the chimeric protein of any one of claims 1-21.

44. The modified cell of any one of claims 41-43, wherein the cell is a mammalian cell.

45. The modified cell of any one of claims 41-44, wherein the cell has been isolated from a mammalian subject.

46. The modified cell of any one of claims 41-45, wherein the cell has been isolated from a human subject.

47. The modified cell of any one of claims 41-46, wherein the cell is a microglia cell.

48. The modified cell of any one of claims 41-46, wherein the cell is an induced pluripotent stem cell (iPSC).

49. The modified cell of any one of claims 41-48, wherein the cell is a microglial cell derived from an iPSC.

50. The modified cell of any one of claims 41-46, wherein the cell is an astrocyte.

51. The modified cell of any one of claims 41-46, wherein the cell is a peripheral monocyte.

52. The modified cell of any one of claims 41-51, wherein the modified cell is capable of phagocytosing an amyloid beta peptide, a tau protein or an α-synuclein protein.

53. A plurality of modified cells of any one of claims 41-52.

54. The plurality of modified cells of claim 53, wherein at least 65%, at least 70%, at least 75%, or at least 80% of the plurality are capable of phagocytosing an amyloid beta peptide, a tau protein or an α-synuclein protein.

55. A pharmaceutical composition comprising the vector of any one of claims 30-36, the modified cell of any one of claims 41-52, the plurality of modified cells of claim 53 or 54, or the rAAV particle of any one of claims 37-39, 72, and 73, and a pharmaceutically acceptable excipient.

56. A nanoparticle comprising the pharmaceutical composition of claim 55 or the chimeric receptor of any one of claims 1-21.

57. A method of treating a subject is diagnosed with or at risk of developing a neurodegenerative disease, disorder, or condition, the method comprising:
administering to the subject the chimeric protein of any one of claims 1-21, the modified cell of any one of claims 41-52, the rAAV particle of any one of claims 37-39, the plurality of modified cells of claim 53 or 54, the pharmaceutical composition of claim 55, or the nanoparticle of claim 56.

58. A method of preventing a neurodegenerative disease, disorder, or condition, the method comprising:
administering to a subject the chimeric protein of any one of claims 1-21, modified cell of any one of claims 41-52, the rAAV particle of any one of claims 37-39, the plurality of modified cells of claim 53 or 54, the pharmaceutical composition of claim 55, or the nanoparticle of claim 56.

59. The method of claim 57 or 58, wherein the disease, disorder, or condition is Alzheimer's Disease or Parkinson's Disease.

60. The method of any one of claims 57-59, wherein the subject is a human.

61. The method of any one of claims 57-60, wherein the modified cells are autologous.

62. The method of any one of claims 57-61, wherein the method comprises administering the modified cells into the brain of the subject.

63. The method of any one of claims 57-62, wherein the step of administering comprises intravenous, intraocular or intracerebroventricular administration.

64. The method of any one of claims 57-63, wherein the step of administering comprises intraocular or intracerebroventricular administration.

65. The method of any one of claims 57-64, wherein the step of administering is performed in vivo in a subject.

66. A method of preparing a modified cell comprising contacting the cell with the polynucleotide of any one of claims 22-29 or the vector of any one of claims 30-36.

67. The method of claim 66, wherein the cell is an astrocyte, a microglia cell, or a peripheral monocyte.

68. The method of claim 66 or 67, wherein the step of contacting is performed in vivo.

69. The method of claim 66 or 67, wherein the step of contacting is performed ex vivo.

70. The chimeric receptor of any one of claims 1-21, or the vector of any one of claims 30-36, for use in treatment of a neurodegenerative disorder.

71. The modified cell of any one of claims 41-52, the plurality of modified cells of claim 53 or 54, the pharmaceutical composition of claim 55, or the nanoparticle of claim 56 for use in treatment of a neurodegenerative disorder.

72. Use of the chimeric receptor of any one of claims 1-21, the vector of any one of claims 30-36, the modified cell of any one of claims 41-52, the plurality of modified cells of claim 53 or 54, the pharmaceutical composition of claim 55, or the nanoparticle of claim 56 in the manufacture of a medicament for the treatment of a neurodegenerative disease in a subject in need thereof.

73. An rAAV particle comprising the vector of any one of claims 30-34 and a PHP.eB capsid.

74. An rAAV particle comprising the vector of any one of claims 30-36, and a TM6 capsid.

75. An rAAV particle comprising the vector of any one of claims 30-34 and a TM2 capsid.


SEQUENCE LISTING (62 sequences)

SEQ ID NO: 1 - protein, 246 aa, Artificial Sequence
  Synthetic
QVTLKESGPGILQPSQTLSLTCSFSGFSLNTFGMGVSWIRQPSGKGLEWLAHIFWDDDKHYNPSLKSRLTISKDTSNNQVFLKITTVDTADTATYYCVRYGFDGFPYWGQGTLVTVSAGGGGSGGGGSGGGGSDVVMTQTPLSLPVSLGDQASISCRSNQSLVHSNGNTYLHWYLQKPGQSPKLLIYKVSNRFSGVPDRFSGSGSGTDFTLKISRVEAEDLGFYFCSQSTRVPWTFGGGTKLELKR

SEQ ID NO: 2 - protein, 502 aa, Artificial Sequence
  Synthetic
TRQLQQSGPELVKPGASGKISCKTSGYTFSENTMHWVKQSHGKSLEWIGGINPNTGATYYNQKFKGKATLTVDTSSSTAYLELRSLTSADSAVYYCVRNGLLTLGAMDYWGQGTDQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRSGGGSSVTVSSGGGGSGGGGSGGGGSDVVMTQTPLSLPVSLGDQASISCRSSQSLVHSNGNTYLYWCLQKPGQSPKLLIYKVSSRFSGVPDRFSGSGSGTDFTLKISRVEAEDLGVYFCSQSTHAPLTFGAGTKLELKRS

SEQ ID NO: 3 - protein, 253 aa, Artificial Sequence
  Synthetic
DQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRSGGGS

SEQ ID NO: 4 - protein, 236 aa, Artificial Sequence
  Synthetic
QVQLQQPGTELVKPGASVKLSCKASGYPFTNYWMHWVKQRPGQGLEWIGNINPTNGDTNYNEKFKSKATLTVDKSSSTAYMQLSSLTSEDSAVYYCTRVTYWGQGTTLTVSSAGGGGSGGGGSGGGGSENVLTQSPAIMSASPGEKVTMTCSASSNVNYMHWFQQESSTSPKLWIYDTSKLASGVPGRFSGSGSGNSYSLTISSMEAEDVATYYCFQGSGYPYTFGGGTKLEIKRL

SEQ ID NO: 5 - protein, 246 aa, Artificial Sequence
  Synthetic
EVQLVQSGGGLVKPGGSLRLSCAASGFTFSSYAMSWVRQAPGKGLEWISSITRGGSYTYFPDSVKGRFTISRDNAKNSLYLQMNSLRAEDTAVYYCARQGNYDGAWCDYWGQGTTVTVSSGGGGSGGGGSGGGGSEIVLTQSPGTLSVSPGERATLSCRASQNIGTSIHWYQQKPGQAPRLLIKYTSESISGIPDRFSGSGSGTDFTLTISRLEPEDFAVYYCHQTKTWPTTFGQGTKVEIKRTLK

SEQ ID NO: 6 - protein, 245 aa, Artificial Sequence
  Synthetic
QVQLQQSGAELARPGASVKLSCKASGYTFTTYWVHWVKQRPGQGLEWIGAIYPGDGDTRYTQKFKGKATLTADESSSTAYMQLSSLASEDSAVYYCARSFAYWGQGTLVTVSAAGGGGSGGGGSGGGGSDIVMTQSPSSLSVSAGEKVTMSCKSSQSLLKSGNQKNYLAWYQQKPGQPPKLLIYGASSRESGVPDRFTGSGSGTDFTLTISSVQAEDLAVYYCQNDHSYPFTFGSGTKLEIKRLK

SEQ ID NO: 7 - protein, 194 aa, Artificial Sequence
  Synthetic
MFPFYSCWRTGLLLLLLAVAVRESWQTTRLKEFDYKDDDDKGDEYDNHCGKEQRRAVVMISCNRHTLADNFNPVSEERGKVQDCFYLFEMDSSLACSPEISHLSVGSILLVTFASLVAVYVVGGFLYQRLVVGAKGMEQFPHLAFWQDLGNLVADGCDFVCRSKPRNVPAAYRGVGDDQLGEESEERDDHLLPM

SEQ ID NO: 8 - protein, 127 aa, Artificial Sequence
  Synthetic
MGGLEPCSRLLLLPLLLAVSGLRTRLKEFDYKDDDDKPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 9 - protein, 130 aa, Artificial Sequence
  Synthetic
MEPLRLLILLFVTELSGAHTRLKEFDYKDDDDKDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 10 - protein, 306 aa, Artificial Sequence
  Synthetic
MSDSKEPRLQQLGLLEEEQLRGLGFRQTRGYKSLAGCLGHGPLVLQLLSFTLLAGLLVQVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTDYKDDDDKGGGGSGGGGSGGGGSLVPSSDPGHLVEFGGSHHHHHH

SEQ ID NO: 11 - protein, 146 aa, Artificial Sequence
  Synthetic
MALPVTALLLPLALLLHAARPTRLKEFDYKDDDDKTTTPAPRPPTPAPTIASQPLSLRPEACRPAAGGAVHTRGLDFACDIYIWAPLAGTCGVLLLSLVITLYCRLKIQVRKAAITSYEKSDGVYTGLSTRNQETYETLKHEKPPQ

SEQ ID NO: 12 - protein, 134 aa, Artificial Sequence
  Synthetic
MRLPLLLVFASVIPGAVLLLTRLKEFDYKDDDDKRPKIIDAKPTHELLTTKADTRKMDPSKPSSNVAGVVIIVILLILTGAGLAAYFFYKKRRVHLPQEGAFENTLYFNSQSSPGTSDMKDLVGNIEQNEHSVI

SEQ ID NO: 13 - protein, 656 aa, Artificial Sequence
  Synthetic
MGPAPLPLLLGLFLPALWRRAITRLKEFDYKDDDDKDIAPSVAPLNVTVFLNESSDNVDIRWMKPPTKQQDGELVGYRISHVWQSAGISKELLEEVGQNGSRARISVQVHNATCTVRIAAVTRGGVGPFSDPVKIFIPAHGWVDYAPSSTPAPGNADPVLIIFGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 14 - protein, 355 aa, Artificial Sequence
  Synthetic
MVISLNSCLSFICLLLCHWITRLKEFDYKDDDDKSPGWKGARCDQAGVIIVGNLNSLSRTSTALPADSYQIGAIAGIIILVLVVLFLLALFIIYRHKQKGKESSMPAVTYTPAMRVVNADYTISGTLPHSNGGNANSHYFTNPSYHTLTQCATSPHVNNRDRMTVTKSKNNQLFVNLKNVNPGKRGPVGDCTGTLPADWKHGGYLNELGAFGLDRSYMGKSLKDLGKNSEYNSSNCSLSSSENPYATIKDPPVLIPKSSECGYVEMKSPARRDSPYAEINNSTSANRNVYEVEPTVSVVQGVFSNNGRLSQDPYDLPKNSHIPCHYDLLPVRDSSSSPKQEDSGGSSSNSSSSSE

SEQ ID NO: 15 - protein, 193 aa, Artificial Sequence
  Synthetic
MALPVTALLLPLALLLHAARPTRLKEFDYKDDDDKTTTPAPRPPTPAPTIASQPLSLRPEACRPAAGGAVHTRGLDFACDIYIWAPLAGTCGVLLLSLVITLYCRLKIQVRKAAITSYEKSDGVYTGLSTRNQETYETLKHEKPPQVSTGGRVHLPQEGAFENTLYFNSQSSPGTSDMKDLVGNIEQNEHSVI

SEQ ID NO: 16 - protein, 181 aa, Artificial Sequence
  Synthetic
MRLPLLLVFASVIPGAVLLLTRLKEFDYKDDDDKRPKIIDAKPTHELLTTKADTRKMDPSKPSSNVAGVVIIVILLILTGAGLAAYFFYKKRRVHLPQEGAFENTLYFNSQSSPGTSDMKDLVGNIEQNEHSVIVSTGGRLKIQVRKAAITSYEKSDGVYTGLSTRNQETYETLKHEKPPQ

SEQ ID NO: 17 - protein, 190 aa, Mus musculus
MFPFSGCWRTELLLLLLLAVAVTRLKEFDYKDDDDKGDEYDNHCGKEQRRAVVMISCNRHTLAANFNPVSEERGKVQDCFYLFEMDSSLACSPEVSHLSVGSILLVIFASLVAVYIIGGFLYQRLVVGAKGMEQFPHLAFWQDLGNLVADGCDFVCRSKPRNVPAAYRGVGDDQLGEESEERDDHLLPMV

SEQ ID NO: 18 - protein, 147 aa, Mus musculus
MALPVTALLLPLALLLHAARPTRLKEFDYKDDDDKTTTPAPRPPTPAPTIASQPLSLRPEACRPAAGGAVHTRGLDFACDIYIWAPLAGTCGVLLLSLVITLYCRLKIQVRKAAIASREKADAVYTGLNTRSQETYETLKHEKPPQV

SEQ ID NO: 19 - protein, 136 aa, Mus musculus
MRLLLLLAFISVIPVSVQLLTRLKEFDYKDDDDKMPKIIDPVTTHSSITTKADQRKMDPQPKGSSKAAGVVTVVLLIVIGAGVAAYFFYKKRHALHIPQEATFENTLYFNSNLSPGTSDTKDLMGNIEQNEHAIIV

SEQ ID NO: 20 - protein, 657 aa, Mus musculus
MVLAPLLLGLLLLPALWSGGTRLKEFDYKDDDDKDIAPSVAPLNITVFLNESNNILDIRWTKPPIKRQDGELVGYRISHVWESAGTYKELSEEVSQNGSWAQIPVQIHNATCTVRIAAITKGGIGPFSEPVNIIIPEDIHSKVDYAPSSTPAPGNTDSMFIILGCFCGFILIGLILCISLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLMV

SEQ ID NO: 21 - protein, 363 aa, Mus musculus
MAISSSSCLGLICSLLCHWVTRLKEFDYKDDDDKSPGWKGARCDQAGVIIVGNLNSLSRTSTALPADSYQIGAIAGIVVLVLVVLFLLALFIIYRHKQKRKESSMPAVTYTPAMRVINADYTIAETLPHSNGGNANSHYFTNPSYHTLSQCATSPHVNNRDRMTIAKSKNNQLFVNLKNVNPGKRGTLVDCTGTLPADWKQGGYLNELGAFGLDRSYMGKSLKDLGKNSEYNSSTCSLSSSENPYATIKDPPALLPKSSECGYVEMKSPARRDSPYAEINNSTPANRNVYEVEPTVSVVQGVFSNSGHVTQDPYDLPKNSHIPCHYDLLPVRDSSSSPKREDGGGSNSTSSNSTSSSSSSSEV

SEQ ID NO: 22 - protein, 129 aa, Mus musculus
MGALEPSWCLLFLPVLLTVGGLSTRLKEFDYKDDDDKPVQAQSDTFPRCDCSSVSPGVLAGIVLGDLVLTLLIALAVYSLGRLVSRGQGTAEGTRKQHIAETESPYQELQGQRPEVYSDLNTQRQYYRV

SEQ ID NO: 23 - protein, 127 aa, Mus musculus
MGPLHQFLLLLITALSQALTRLKEFDYKDDDDKDDQDAGDLWVPEESSSFEGAQVEHSTSRNQETSFPPTSILLLLACVLLSKFLAASILWAVARGRQKPGTPVVRGLDCGQDAGHQLQILTGPGGT

SEQ ID NO: 24 - protein, 195 aa, Mus musculus
MALPVTALLLPLALLLHAARPTRLKEFDYKDDDDKTTTPAPRPPTPAPTIASQPLSLRPEACRPAAGGAVHTRGLDFACDIYIWAPLAGTCGVLLLSLVITLYCRLKIQVRKAAIASREKADAVYTGLNTRSQETYETLKHEKPPQVSTGGHALHIPQEATFENTLYFNSNLSPGTSDTKDLMGNIEQNEHAIIV

SEQ ID NO: 25 - protein, 184 aa, Mus musculus
MALPVTALLLPLALLLHAARPTRLKEFDYKDDDDKMPKIIDPVTTHSSITTKADQRKMDPQPKGSSKAAGVVTVVLLIVIGAGVAAYFFYKKRHALHIPQEATFENTLYFNSNLSPGTSDTKDLMGNIEQNEHAIIVSTGGRLKIQVRKAAIASREKADAVYTGLNTRSQETYETLKHEKPPQV

SEQ ID NO: 26 - protein, 376 aa, Artificial Sequence
  Synthetic
MEPLRLLILLFVTELSGAHTRQVTLKESGPGILQPSQTLSLTCSFSGFSLNTFGMGVSWIRQPSGKGLEWLAHIFWDDDKHYNPSLKSRLTISKDTSNNQVFLKITTVDTADTATYYCVRYGFDGFPYWGQGTLVTVSAGGGGSGGGGSGGGGSDVVMTQTPLSLPVSLGDQASISCRSNQSLVHSNGNTYLHWYLQKPGQSPKLLIYKVSNRFSGVPDRFSGSGSGTDFTLKISRVEAEDLGFYFCSQSTRVPWTFGGGTKLELKRLKEFDYKDDDDKDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 27 - protein, 552 aa, Artificial Sequence
  Synthetic
MSDSKEPRLQQLGLLEEEQLRGLGFRQTRGYKSLAGCLGHGPLVLQLLSFTLLAGLLVQVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTDYKDDDDKGGGGSGGGGSGGGGSLVPSSDPGHLVEFQVTLKESGPGILQPSQTLSLTCSFSGFSLNTFGMGVSWIRQPSGKGLEWLAHIFWDDDKHYNPSLKSRLTISKDTSNNQVFLKITTVDTADTATYYCVRYGFDGFPYWGQGTLVTVSAGGGGSGGGGSGGGGSDVVMTQTPLSLPVSLGDQASISCRSNQSLVHSNGNTYLHWYLQKPGQSPKLLIYKVSNRFSGVPDRFSGSGSGTDFTLKISRVEAEDLGFYFCSQSTRVPWTFGGGTKLELKRGGSHHHHHH

SEQ ID NO: 28 - protein, 392 aa, Artificial Sequence
  Synthetic
MALPVTALLLPLALLLHAARPTRQVTLKESGPGILQPSQTLSLTCSFSGFSLNTFGMGVSWIRQPSGKGLEWLAHIFWDDDKHYNPSLKSRLTISKDTSNNQVFLKITTVDTADTATYYCVRYGFDGFPYWGQGTLVTVSAGGGGSGGGGSGGGGSDVVMTQTPLSLPVSLGDQASISCRSNQSLVHSNGNTYLHWYLQKPGQSPKLLIYKVSNRFSGVPDRFSGSGSGTDFTLKISRVEAEDLGFYFCSQSTRVPWTFGGGTKLELKRLKEFDYKDDDDKTTTPAPRPPTPAPTIASQPLSLRPEACRPAAGGAVHTRGLDFACDIYIWAPLAGTCGVLLLSLVITLYCRLKIQVRKAAITSYEKSDGVYTGLSTRNQETYETLKHEKPPQ

SEQ ID NO: 29 - protein, 380 aa, Artificial Sequence
  Synthetic
MRLPLLLVFASVIPGAVLLLTRQVTLKESGPGILQPSQTLSLTCSFSGFSLNTFGMGVSWIRQPSGKGLEWLAHIFWDDDKHYNPSLKSRLTISKDTSNNQVFLKITTVDTADTATYYCVRYGFDGFPYWGQGTLVTVSAGGGGSGGGGSGGGGSDVVMTQTPLSLPVSLGDQASISCRSNQSLVHSNGNTYLHWYLQKPGQSPKLLIYKVSNRFSGVPDRFSGSGSGTDFTLKISRVEAEDLGFYFCSQSTRVPWTFGGGTKLELKRLKEFDYKDDDDKRPKIIDAKPTHELLTTKADTRKMDPSKPSSNVAGVVIIVILLILTGAGLAAYFFYKKRRVHLPQEGAFENTLYFNSQSSPGTSDMKDLVGNIEQNEHSVI

SEQ ID NO: 30 - protein, 902 aa, Artificial Sequence
  Synthetic
MGPAPLPLLLGLFLPALWRRAITRQVTLKESGPGILQPSQTLSLTCSFSGFSLNTFGMGVSWIRQPSGKGLEWLAHIFWDDDKHYNPSLKSRLTISKDTSNNQVFLKITTVDTADTATYYCVRYGFDGFPYWGQGTLVTVSAGGGGSGGGGSGGGGSDVVMTQTPLSLPVSLGDQASISCRSNQSLVHSNGNTYLHWYLQKPGQSPKLLIYKVSNRFSGVPDRFSGSGSGTDFTLKISRVEAEDLGFYFCSQSTRVPWTFGGGTKLELKRLKEFDYKDDDDKDIAPSVAPLNVTVFLNESSDNVDIRWMKPPTKQQDGELVGYRISHVWQSAGISKELLEEVGQNGSRARISVQVHNATCTVRIAAVTRGGVGPFSDPVKIFIPAHGWVDYAPSSTPAPGNADPVLIIFGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 31 - protein, 601 aa, Artificial Sequence
  Synthetic
MVISLNSCLSFICLLLCHWITRQVTLKESGPGILQPSQTLSLTCSFSGFSLNTFGMGVSWIRQPSGKGLEWLAHIFWDDDKHYNPSLKSRLTISKDTSNNQVFLKITTVDTADTATYYCVRYGFDGFPYWGQGTLVTVSAGGGGSGGGGSGGGGSDVVMTQTPLSLPVSLGDQASISCRSNQSLVHSNGNTYLHWYLQKPGQSPKLLIYKVSNRFSGVPDRFSGSGSGTDFTLKISRVEAEDLGFYFCSQSTRVPWTFGGGTKLELKRLKEFDYKDDDDKSPGWKGARCDQAGVIIVGNLNSLSRTSTALPADSYQIGAIAGIIILVLVVLFLLALFIIYRHKQKGKESSMPAVTYTPAMRVVNADYTISGTLPHSNGGNANSHYFTNPSYHTLTQCATSPHVNNRDRMTVTKSKNNQLFVNLKNVNPGKRGPVGDCTGTLPADWKHGGYLNELGAFGLDRSYMGKSLKDLGKNSEYNSSNCSLSSSENPYATIKDPPVLIPKSSECGYVEMKSPARRDSPYAEINNSTSANRNVYEVEPTVSVVQGVFSNNGRLSQDPYDLPKNSHIPCHYDLLPVRDSSSSPKQEDSGGSSSNSSSSSE

SEQ ID NO: 32 - protein, 439 aa, Artificial Sequence
  Synthetic
MALPVTALLLPLALLLHAARPTRQVTLKESGPGILQPSQTLSLTCSFSGFSLNTFGMGVSWIRQPSGKGLEWLAHIFWDDDKHYNPSLKSRLTISKDTSNNQVFLKITTVDTADTATYYCVRYGFDGFPYWGQGTLVTVSAGGGGSGGGGSGGGGSDVVMTQTPLSLPVSLGDQASISCRSNQSLVHSNGNTYLHWYLQKPGQSPKLLIYKVSNRFSGVPDRFSGSGSGTDFTLKISRVEAEDLGFYFCSQSTRVPWTFGGGTKLELKRLKEFDYKDDDDKTTTPAPRPPTPAPTIASQPLSLRPEACRPAAGGAVHTRGLDFACDIYIWAPLAGTCGVLLLSLVITLYCRLKIQVRKAAITSYEKSDGVYTGLSTRNQETYETLKHEKPPQVSTGGRVHLPQEGAFENTLYFNSQSSPGTSDMKDLVGNIEQNEHSVI

SEQ ID NO: 33 - protein, 427 aa, Artificial Sequence
  Synthetic
MRLPLLLVFASVIPGAVLLLTRQVTLKESGPGILQPSQTLSLTCSFSGFSLNTFGMGVSWIRQPSGKGLEWLAHIFWDDDKHYNPSLKSRLTISKDTSNNQVFLKITTVDTADTATYYCVRYGFDGFPYWGQGTLVTVSAGGGGSGGGGSGGGGSDVVMTQTPLSLPVSLGDQASISCRSNQSLVHSNGNTYLHWYLQKPGQSPKLLIYKVSNRFSGVPDRFSGSGSGTDFTLKISRVEAEDLGFYFCSQSTRVPWTFGGGTKLELKRLKEFDYKDDDDKRPKIIDAKPTHELLTTKADTRKMDPSKPSSNVAGVVIIVILLILTGAGLAAYFFYKKRRVHLPQEGAFENTLYFNSQSSPGTSDMKDLVGNIEQNEHSVIVSTGGRLKIQVRKAAITSYEKSDGVYTGLSTRNQETYETLKHEKPPQ

SEQ ID NO: 34 - protein, 379 aa, Artificial Sequence
  Synthetic
MEPLRLLILLFVTELSGAHTRTRQLQQSGPELVKPGASGKISCKTSGYTFSENTMHWVKQSHGKSLEWIGGINPNTGATYYNQKFKGKATLTVDTSSSTAYLELRSLTSADSAVYYCVRNGLLTLGAMDYWGQGTSVTVSSGGGGSGGGGSGGGGSDVVMTQTPLSLPVSLGDQASISCRSSQSLVHSNGNTYLYWCLQKPGQSPKLLIYKVSSRFSGVPDRFSGSGSGTDFTLKISRVEAEDLGVYFCSQSTHAPLTFGAGTKLELKRSLKEFDYKDDDDKDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 35 - protein, 395 aa, Artificial Sequence
  Synthetic
MALPVTALLLPLALLLHAARPTRTRQLQQSGPELVKPGASGKISCKTSGYTFSENTMHWVKQSHGKSLEWIGGINPNTGATYYNQKFKGKATLTVDTSSSTAYLELRSLTSADSAVYYCVRNGLLTLGAMDYWGQGTSVTVSSGGGGSGGGGSGGGGSDVVMTQTPLSLPVSLGDQASISCRSSQSLVHSNGNTYLYWCLQKPGQSPKLLIYKVSSRFSGVPDRFSGSGSGTDFTLKISRVEAEDLGVYFCSQSTHAPLTFGAGTKLELKRSLKEFDYKDDDDKTTTPAPRPPTPAPTIASQPLSLRPEACRPAAGGAVHTRGLDFACDIYIWAPLAGTCGVLLLSLVITLYCRLKIQVRKAAITSYEKSDGVYTGLSTRNQETYETLKHEKPPQ

SEQ ID NO: 36 - protein, 366 aa, Artificial Sequence
  Synthetic
MEPLRLLILLFVTELSGAHTRQVQLQQPGTELVKPGASVKLSCKASGYPFTNYWMHWVKQRPGQGLEWIGNINPTNGDTNYNEKFKSKATLTVDKSSSTAYMQLSSLTSEDSAVYYCTRVTYWGQGTTLTVSSAGGGGSGGGGSGGGGSENVLTQSPAIMSASPGEKVTMTCSASSNVNYMHWFQQESSTSPKLWIYDTSKLASGVPGRFSGSGSGNSYSLTISSMEAEDVATYYCFQGSGYPYTFGGGTKLEIKRLLKEFDYKDDDDKDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 37 - protein, 382 aa, Artificial Sequence
  Synthetic
MALPVTALLLPLALLLHAARPTRQVQLQQPGTELVKPGASVKLSCKASGYPFTNYWMHWVKQRPGQGLEWIGNINPTNGDTNYNEKFKSKATLTVDKSSSTAYMQLSSLTSEDSAVYYCTRVTYWGQGTTLTVSSAGGGGSGGGGSGGGGSENVLTQSPAIMSASPGEKVTMTCSASSNVNYMHWFQQESSTSPKLWIYDTSKLASGVPGRFSGSGSGNSYSLTISSMEAEDVATYYCFQGSGYPYTFGGGTKLEIKRLLKEFDYKDDDDKTTTPAPRPPTPAPTIASQPLSLRPEACRPAAGGAVHTRGLDFACDIYIWAPLAGTCGVLLLSLVITLYCRLKIQVRKAAITSYEKSDGVYTGLSTRNQETYETLKHEKPPQ

SEQ ID NO: 38 - protein, 376 aa, Artificial Sequence
  Synthetic
MEPLRLLILLFVTELSGAHTREVQLVQSGGGLVKPGGSLRLSCAASGFTFSSYAMSWVRQAPGKGLEWISSITRGGSYTYFPDSVKGRFTISRDNAKNSLYLQMNSLRAEDTAVYYCARQGNYDGAWCDYWGQGTTVTVSSGGGGSGGGGSGGGGSEIVLTQSPGTLSVSPGERATLSCRASQNIGTSIHWYQQKPGQAPRLLIKYTSESISGIPDRFSGSGSGTDFTLTISRLEPEDFAVYYCHQTKTWPTTFGQGTKVEIKRTLKLKEFDYKDDDDKDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 39 - protein, 392 aa, Artificial Sequence
  Synthetic
MALPVTALLLPLALLLHAARPTREVQLVQSGGGLVKPGGSLRLSCAASGFTFSSYAMSWVRQAPGKGLEWISSITRGGSYTYFPDSVKGRFTISRDNAKNSLYLQMNSLRAEDTAVYYCARQGNYDGAWCDYWGQGTTVTVSSGGGGSGGGGSGGGGSEIVLTQSPGTLSVSPGERATLSCRASQNIGTSIHWYQQKPGQAPRLLIKYTSESISGIPDRFSGSGSGTDFTLTISRLEPEDFAVYYCHQTKTWPTTFGQGTKVEIKRTLKLKEFDYKDDDDKTTTPAPRPPTPAPTIASQPLSLRPEACRPAAGGAVHTRGLDFACDIYIWAPLAGTCGVLLLSLVITLYCRLKIQVRKAAITSYEKSDGVYTGLSTRNQETYETLKHEKPPQ

SEQ ID NO: 40 - protein, 375 aa, Artificial Sequence
  Synthetic
MEPLRLLILLFVTELSGAHTRQVQLQQSGAELARPGASVKLSCKASGYTFTTYWVHWVKQRPGQGLEWIGAIYPGDGDTRYTQKFKGKATLTADESSSTAYMQLSSLASEDSAVYYCARSFAYWGQGTLVTVSAAGGGGSGGGGSGGGGSDIVMTQSPSSLSVSAGEKVTMSCKSSQSLLKSGNQKNYLAWYQQKPGQPPKLLIYGASSRESGVPDRFTGSGSGTDFTLTISSVQAEDLAVYYCQNDHSYPFTFGSGTKLEIKRLKLKEFDYKDDDDKDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 41 - protein, 391 aa, Artificial Sequence
  Synthetic
MALPVTALLLPLALLLHAARPTRQVQLQQSGAELARPGASVKLSCKASGYTFTTYWVHWVKQRPGQGLEWIGAIYPGDGDTRYTQKFKGKATLTADESSSTAYMQLSSLASEDSAVYYCARSFAYWGQGTLVTVSAAGGGGSGGGGSGGGGSDIVMTQSPSSLSVSAGEKVTMSCKSSQSLLKSGNQKNYLAWYQQKPGQPPKLLIYGASSRESGVPDRFTGSGSGTDFTLTISSVQAEDLAVYYCQNDHSYPFTFGSGTKLEIKRLKLKEFDYKDDDDKTTTPAPRPPTPAPTIASQPLSLRPEACRPAAGGAVHTRGLDFACDIYIWAPLAGTCGVLLLSLVITLYCRLKIQVRKAAITSYEKSDGVYTGLSTRNQETYETLKHEKPPQ

SEQ ID NO: 42 - protein, 383 aa, Artificial Sequence
  Synthetic
MEPLRLLILLFVTELSGAHTRDQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRSGGGSLKEFDYKDDDDKDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 43 - protein, 399 aa, Artificial Sequence
  Synthetic
MALPVTALLLPLALLLHAARPTRDQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRSGGGSLKEFDYKDDDDKTTTPAPRPPTPAPTIASQPLSLRPEACRPAAGGAVHTRGLDFACDIYIWAPLAGTCGVLLLSLVITLYCRLKIQVRKAAITSYEKSDGVYTGLSTRNQETYETLKHEKPPQ

SEQ ID NO: 44 - protein, 8 aa, Artificial Sequence
  Synthetic
DYKDDDDK

SEQ ID NO: 45 - DNA, 441 bp, Artificial Sequence
  Synthetic
atggccttaccagtgaccgccttgctcctgccgctggccttgctgctccacgccgccaggccgacgcgtcttaaggaattcgattacaaggatgacgacgataagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccgactgaagatccaagtgcgaaaggcagctataaccagctatgagaaatcagatggtgtttacacgggcctgagcaccaggaaccaggagacttacgagactctgaagcatgagaaaccaccacaggtt

SEQ ID NO: 46 - DNA, 405 bp, Artificial Sequence
  Synthetic
atgaggctacccctgctcctggtttttgcctctgtcattccgggtgctgttctcctactgacgcgtcttaaggaattcgattacaaggatgacgacgataagagaccaaaaattattgatgctaaacctactcatgaattacttacaacaaaagctgacacaaggaagatggacccttctaaaccgtcttccaacgtggccggagtagtcatcattgtgatcctcctgattttaacgggtgctggccttgccgcctatttcttttataagaaaagacgtgtgcacctacctcaagagggcgcctttgaaaacactctgtattttaacagtcagtcaagcccaggaactagtgatatgaaagatctcgtgggcaatattgaacagaatgaacactcggtcatcgtt

SEQ ID NO: 47 - DNA, 1968 bp, Artificial Sequence
  Synthetic
atggggccggccccgctgccgctgctgctgggcctcttcctccccgcgctctggcgtagagctatcacgcgtcttaaggaattcgattacaaggatgacgacgataaggatgccccatcagtagcacctttaaatgtcactgtgtttctgaatgaatctagtgataatgtggacatcagatggatgaagcctccgactaagcagcaggatggagaactggtgggctaccggatatcccacgtgtggcagagtgcagggatttccaaagagctcttggaggaagttggccagaatggcagccgagctcggatctctgttcaagtccacaatgctacgtgcacagtgaggattgcagccgtcaccagagggggagttgggcccttcagtgatccagtgaaaatatttatccctgcacacggttgggtagattatgccccctcttcaactccggcgcctggcaacgcagatcctgtgctcatcatctttggctgcttttgtggatttattttgattgggttgattttatacatctccttggccatcagaaaaagagtccaggagacaaagtttgggaatgcattcacagaggaggattctgaattagtggtgaattatatagcaaagaaatccttctgtcggcgagccattgaacttaccttacatagcttgggagtcagtgaggaactacaaaataaactagaagatgttgtgattgacaggaatcttctaattcttggaaaaattctgggtgaaggagagtttgggtctgtaatggaaggaaatcttaagcaggaagatgggacctctctgaaagtggcagtgaagaccatgaagttggacaactcttcacagcgggagatcgaggagtttctcagtgaggcagcgtgcatgaaagacttcagccacccaaatgtcattcgacttctaggtgtgtgtatagaaatgagctctcaaggcatcccaaagcccatggtaattttacccttcatgaaatacggggacctgcatacttacttactttattcccgattggagacaggaccaaagcatattcctctgcagacactattgaagttcatggtggatattgccctgggaatggagtatctgagcaacaggaattttcttcatcgagatttagctgctcgaaactgcatgttgcgagatgacatgactgtctgtgttgcggacttcggcctctctaagaagatttacagtggcgattattaccgccaaggccgcattgctaagatgcctgttaaatggatcgccatagaaagtcttgcagaccgagtctacacaagtaaaagtgatgtgtgggcatttggcgtgaccatgtgggaaatagctacgcggggaatgactccctatcctggggtccagaaccatgagatgtatgactatcttctccatggccacaggttgaagcagcccgaagactgcctggatgaactgtatgaaataatgtactcttgctggagaaccgatcccttagaccgccccaccttttcagtattgaggctgcagctagaaaaactcttagaaagtttgcctgacgttcggaaccaagcagacgttatttacgtcaatacacagttgctggagagctctgagggcctggcccagggctccacccttgctccactggacttgaacatcgaccctgactctataattgcctcctgcactccccgcgctgccatcagtgtggtcacagcagaagttcatgacagcaaacctcatgaaggacggtacatcctgaatgggggcagtgaggaatgggaagatctgacttctgccccctctgctgcagtcacagctgaaaagaacagtgttttaccgggggagagacttgttaggaatggggtctcctggtcccattcgagcatgctgcccttgggaagctcattgcccgatgaacttttgtttgctgacgactcctcagaaggctcagaagtcctgatggtt

SEQ ID NO: 48 - DNA, 1068 bp, Artificial Sequence
  Synthetic
atggttatttctttgaactcatgcctgagctttatttgtttattgttatgccactggattacgcgtcttaaggaattcgattacaaggatgacgacgataagagccccggatggaagggagcgagatgtgatcaagctggtgttatcatagttggaaatctgaacagcttaagccgaaccagtactgctctccctgctgattcctaccagatcggggccattgcaggcatcatcattcttgtcctagttgttctcttcctactggcattgttcattatttatagacacaagcagaagggaaaggaatcaagcatgccagcagttacctacacccctgctatgagggtcgtcaatgcagattataccatttcaggaacccttcctcacagcaatggtggaaacgctaatagccactacttcaccaatcccagttaccacacgctcacccagtgtgccacatcccctcacgtcaacaacagggacaggatgactgtcacgaagtcaaaaaacaatcaactgtttgtgaatcttaaaaatgtgaaccctgggaagagaggccctgtgggggactgcactgggacattgccggctgactggaaacatggcggctacctcaacgagctcggtgcttttggacttgacagaagctatatgggaaaatccttaaaagacctgggaaagaattctgaatataattcaagtaactgctccctaagcagttctgagaacccatatgccactattaaagacccacctgtacttatcccgaaaagctcagagtgtggttatgtggagatgaaatcgccggcacgaagagattccccatatgcagagatcaataactcaacttcagccaacaggaatgtctatgaagttgaacctacagtgagtgttgtccaaggagtattcagcaataatgggcgtctctcccaggatccatatgacctcccaaagaacagtcacatcccttgtcattatgacctgctgccagtccgagacagttcatcctcccctaagcaagaggacagtggtggtagcagcagcaacagcagcagcagcagtgaagtt

SEQ ID NO: 49 - DNA, 384 bp, Artificial Sequence
  Synthetic
atggggggacttgaaccctgcagcaggctcctgctcctgcctctcctgctggctgtaagtggtctccgtacgcgtcttaaggaattcgattacaaggatgacgacgataagcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaagtt

SEQ ID NO: 50 - DNA, 390 bp, Artificial Sequence
  Synthetic
atggagcctctccggctgctcatcttactctttgtcacagagctgtccggagcccacacgcgtcttaaggaattcgattacaaggatgacgacgataaggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacg

SEQ ID NO: 51 - DNA, 582 bp, Artificial Sequence
  Synthetic
atggccttaccagtgaccgccttgctcctgccgctggccttgctgctccacgccgccaggccgacgcgtcttaaggaattcgattacaaggatgacgacgataagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccgactgaagatccaagtgcgaaaggcagctataaccagctatgagaaatcagatggtgtttacacgggcctgagcaccaggaaccaggagacttacgagactctgaagcatgagaaaccaccacaggtttcgaccggtggccgtgtgcacctacctcaagagggcgcctttgaaaacactctgtattttaacagtcagtcaagcccaggaactagtgatatgaaagatctcgtgggcaatattgaacagaatgaacactcggtcatcgtt

SEQ ID NO: 52 - DNA, 546 bp, Artificial Sequence
  Synthetic
atgaggctacccctgctcctggtttttgcctctgtcattccgggtgctgttctcctactgacgcgtcttaaggaattcgattacaaggatgacgacgataagagaccaaaaattattgatgctaaacctactcatgaattacttacaacaaaagctgacacaaggaagatggacccttctaaaccgtcttccaacgtggccggagtagtcatcattgtgatcctcctgattttaacgggtgctggccttgccgcctatttcttttataagaaaagacgtgtgcacctacctcaagagggcgcctttgaaaacactctgtattttaacagtcagtcaagcccaggaactagtgatatgaaagatctcgtgggcaatattgaacagaatgaacactcggtcatcgtttcgaccggtggccgactgaagatccaagtgcgaaaggcagctataaccagctatgagaaatcagatggtgtttacacgggcctgagcaccaggaaccaggagacttacgagactctgaagcatgagaaaccaccacaggtt

SEQ ID NO: 53 - DNA, 585 bp, Artificial Sequence
  Synthetic
atgttccctttctacagctgctggaggactggactgctactactactcctggctgtggcagtgagagaatcctggcagacaacgcgtcttaaggaattcgattacaaggatgacgacgataagggtgatgaatatgacaaccactgtggcaaggagcagcgtcgtgcagtggtgatgatctcctgcaatcgacacaccctagcggacaattttaaccctgtgtctgaggagcgtggcaaagtccaagattgtttctacctctttgagatggatagcagcctggcctgttcaccagagatctcccacctcagtgtgggttccatcttacttgtcacgtttgcatcactggttgctgtttatgttgttggggggttcctataccagcgactggtagtgggagccaaaggaatggagcagtttccccacttagccttctggcaggatcttggcaacctggtagcagatggctgtgactttgtctgccgttctaaacctcgaaatgtgcctgcagcatatcgtggtgtgggggatgaccagctgggggaggagtcagaagaaagggatgaccatttattaccaatggtt

SEQ ID NO: 54 - DNA, 921 bp, Artificial Sequence
  Synthetic
atgagtgactccaaggaaccaagactgcagcagctgggcctcctggaggaggaacagctgagaggccttggattccgacagactcggggatacaagagcttagcagggtgtcttggccatggtcccctggtgctgcaactcctctccttcacgctcttggctgggctccttgtccaagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacagattacaaggatgacgacgataagggtggcggcggtagcggtggtggcggtagcggcggtggtggcagcttggtaccgagctcggatccaggtcacctggtggaattcggaggaagccatcatcatcatcatcattga

SEQ ID NO: 55 - protein, 4 aa, Artificial Sequence
  Synthetic
GGGS

SEQ ID NO: 56 - protein, 8 aa, Artificial Sequence
  Synthetic
GGGSGGGS

SEQ ID NO: 57 - protein, 121 aa, Artificial Sequence
  Synthetic
MRLLLLLAFISVIPVSVQLLMPKIIDPVTTHSSITTKADQRKMDPQPKGSSKAAGVVTVVLLIVIGAGVAAYFFYKKRHALHIPQEATFENTLYFNSNLSPGTSDTKDLMGNIEQNEHAII

SEQ ID NO: 58 - protein, 69 aa, Artificial Sequence
  Synthetic
MAISSSSCLGLICSLLCHWVSPGWKGARCDQAGVIIVGNLNSLSRTSTALPADSYQISNSTSSSSSSSE

SEQ ID NO: 59 - protein, 114 aa, Artificial Sequence
  Synthetic
MGALEPSWCLLFLPVLLTVGGLSPVQAQSDTFPRCDCSSVSPGVLAGIVLGDLVLTLLIALAVYSLGRLVSRGQGTAEGTRKQHIAETESPYQELQGQRPEVYSDLNTQRQYYR

SEQ ID NO: 60 - protein, 136 aa, Artificial Sequence
  Synthetic
MVLAPLLLGLLLLPALWSGGAPLNITVFLNESNNILDIRWTKPPIKRQDGELVGYRISHVWESAGTYKELSEEVSQNGSWAQIPVQIHNATCTVRIAAITKGGIGPFSEPVNIIIPEHSKVDYAPDDSLEDSEVLM

SEQ ID NO: 61 - protein, 132 aa, Artificial Sequence
  Synthetic
MALPVTALLLPLALLLHAARPTTTPAPRPPTPAPTIASQPLSLRPEACRPAAGGAVHTRGLDFACDIYIWAPLAGTCGVLLLSLVITLYCRLKIQVRKAAIASREKADAVYTGLNTRSQETYETLKHEKPPQ

SEQ ID NO: 62 - protein, 22 aa, Artificial Sequence
  Synthetic
MSDSKEPRLQERLCHPCPWEWT